(19) United States
(12) Patent Application Publication
(10) Pub. No.: US 2010/0303746 A1
(43) Pub. Date: Dec. 2, 2010

(54) GRINDING AIDES FOR MICRONIZED ORGANIC UV ABSORBERS

(75) Inventors: Sebastien Mongiat, Sierentz (FR); Julie Grumelard, Huningue (FR); Thomas Ehlis, Freiburg (DE)

Correspondence Address: BASF Corporation;Patent Department, 500 White Plains Road, P.O. Box 2005, Tarrytown, NY, 10591, US

(73) Assignee: BASF SE, Tarrytown, NY (US)

(21) Appl. No.: 12/744,495

(22) PCT Filed: Nov. 20, 2008
(86) PCT No.: PCT/EP08/65916

§ 371 Date: Aug. 20, 2010

(30) Foreign Application Priority Data
Nov. 29, 2007 (EP) 07121894.5

(51) Int. Cl. A61K 8/49; A61Q 17/04

(52) U.S. Cl. 424/60; 424/59

(57) ABSTRACT

A method of preparing a composition, comprising (a) a micronised insoluble organic UV absorber, which method comprises grinding the insoluble organic UV absorber, in coarse particle form, in a grinding apparatus, in the presence of a grinding aid selected from the groups (b₁) carboxylic acids and their salts; (b₂) fatty acid esters (b₃) alkyl phosphates or phosphoric acid esters; (b₄) ethoxylated carboxylic acids or polyethyleneglycol (PEG) esters; (b₅) fatty alcohol polyethyleneglycol (PEG) ethers of fatty alcohols; (b₆) polyethylene/polypropylene -glycol ethers of saturated or unsaturated C₄-C₂₈ fatty acids; (b₇) ethoxylated alkylphenols or ethoxylated alkylphenyl ethers; (b₈) esters of polyol and mono-, di- or tri-glycerides; (b₉) esters of fatty acids and saccharose; (b₁₀) sorbitan mono- and di-esters of saturated and/or unsaturated C₆-C₂₂ fatty acids and ethylene oxide groups; and (b₁₁) surfactants which are mainly acting as detergent or cleansing agents.

The present invention relates to a method for producing new formulations of UV absorbers and to their use in sunscreen compositions which, in turn, are useful, in particular, for the protection of human skin.

It has long been known that prolonged exposure to that UV radiation which reaches the surface of the earth can lead to the formation of erythemas or light dermatoses, as well as to an increased incidence of skin cancers or accelerated skin aging.

Various sunscreen formulations have been proposed which include a material which is intended to counteract UV radiation, thereby inhibiting the said undesired effects on the skin.

A great number of compounds has been proposed for use as UV protectants in sunscreen formulations, especially soluble organic UV absorbers and insoluble micronised inorganic compounds, in particular zinc oxide and titanium dioxide.

The high specific weight of insoluble inorganic compounds, such as zinc oxide and titanium dioxide leads to a reduced stability of formulations containing them. Moreover, such inorganic compounds have been claimed to generate toxic radicals under the influence of light (“Redox Mechanisms in Heterogeneous Photocatalysis”, Serpone et al, Electrochemistry in Colloids and Dispersions, Editors Mackay and Texter, VCH Publishers Inc., New York 1992).

Micronised, insoluble organic UV absorbers, when used in sunscreen formulations, provide excellent UV protection and have a high SPF rating. Moreover, micronised, insoluble organic UV absorbers show no tendency, under the influence of light, to generate radicals which could damage or sensitise human skin.

Accordingly, the present invention provides a method of preparing a composition, comprising (a) a micronised insoluble organic UV absorber, which method comprises grinding the insoluble organic UV absorber, in coarse particle form, in a grinding apparatus, in the presence of a grinding aid (b) selected from the groups

(b1) carboxylic acids and their salts;

(b₂) fatty acid esters

(b₃) alkyl phosphates or phosphoric acid esters;

(b₄) ethoxylated carboxylic acids or polyethyleneglycol (PEG) esters;

(b₅) fatty alcohol polyethyleneglycol (PEG) ethers of fatty alcohols;

(b₆) polyethylene/polypropylene -glycol ethers of saturated or unsaturated C₄-C₂₈ fatty acids;

(b₇) ethoxylated alkylphenols or ethoxylated alkylphenyl ethers;

(b₈) esters of polyol and mono-, di- or tri-glycerides;

(b₉) esters of fatty acids and saccharose;

(b₁₀) sorbitan mono- and di-esters of saturated and/or unsaturated C₆-C₂₂ fatty acids and ethylene oxide groups; and

(b₁₁) surfactants which are mainly acting as detergent or cleansing agents.

Preferably the micronised insoluble organic UV absorber is selected from the compounds of formula

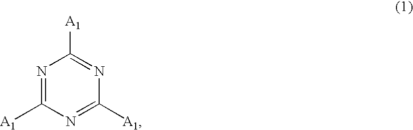

wherein

A is a radical of formula

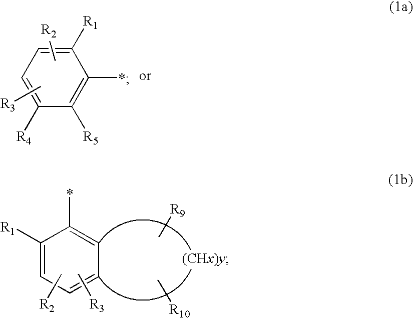

R₁ and R₅ independently from each other are hydrogen; C₁-C₁₈alkyl; or C₆-C₁₂aryl;

R₂, R₃ and R₄ independently from each other are hydrogen; or a radical of formula

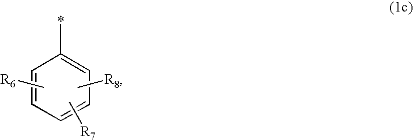

wherein at least one of the radicals R₂, R₃ and R₄ are a radical of formula (1c);

R₆, R₇, R₈, R₉ and R₁₀ independently from each other are hydrogen; hydroxy; halogen; C₁-C₁₈alkyl; C₁-C₁₈alkoxy; C ₆-C₁₂aryl; biphenylyl; C₆-C₁₂aryloxy; C₁-C₁₈alkylthio; carboxy; —COOM; C₁-C₁₈-alkylcarboxyl; aminocarbonyl; or mono- or di-C₁-C₁₈alkylamino; C₁-C₁₀acylamino; —COOH;

M is an alkali metal ion;

x is 1 or 2; and

y is a number from 2 to 10.

More preferably the insoluble UV absorber is selected from the compounds of formula

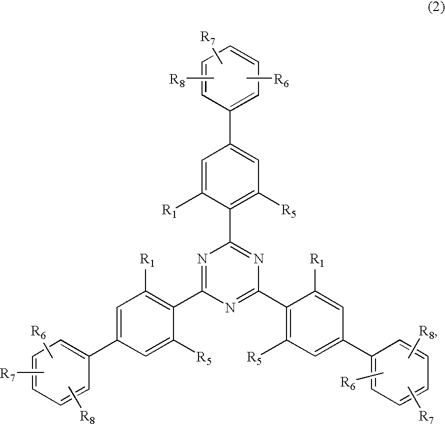

wherein

R₁, R₅, R₆, R₇ and R₈ are defined as in formula (1), and preferably R₁ and R₅ are hydrogen.

Preferably in formulas (1) and (2) R₆ and R₈ are hydrogen; and

R₇ is hydrogen; hydroxy; C₁-C₅alkyl; C₁-C₅alkoxy; —COOM; —COOH; or COOR₁₀;

M is an alkali metal ion; and

R₁₀ is C₁-C₅alkyl.

Most preferred in the method of the present invention are the compounds of formula

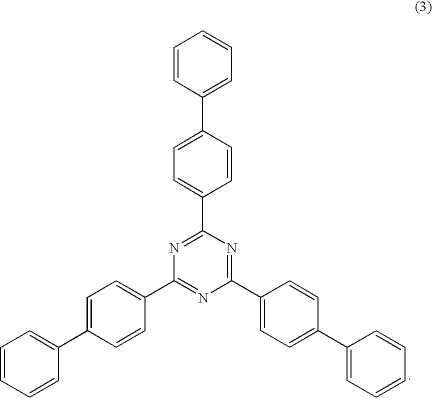

Furthermore, the micronized insoluble UV absorber used in the present invention is selected from the compounds of formula

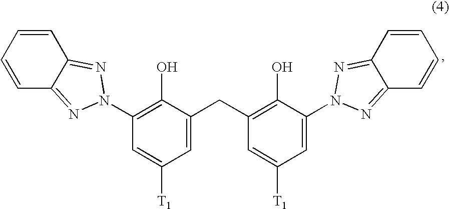

wherein

T₁ is C₁-C₁₈alkyl, which is optionally substituted by phenyl; and more preferably C₁-C₈alkyl.

Most preferred are the micronized UV absorbers of formula

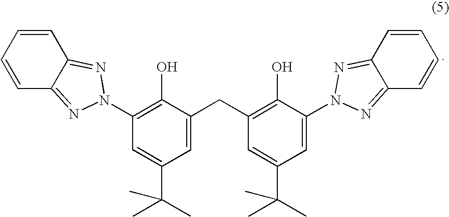

Furthermore, the micronized insoluble UV absorber used in the present invention is selected from the compounds of formula

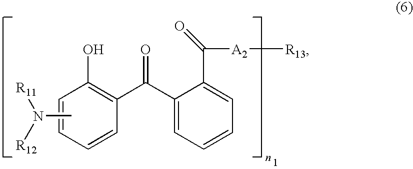

wherein

R₁₁ and R₁₂ independently from each other are C₁-C₂₀alkyl; C₂-C₂₀alkenyl; C₃-C₁₀cycloalkyl; C₃-C₁₀cycloalkenyl; or R₁₁ and R₁₂ together with the linking nitrogen atom form a 5- or 6-membered heterocyclic ring;

n₁ is a number from 1 to 4;

when n₁=1,

R₁₃ is a saturated or unsaturated heterocyclic radical; hydroxy-C₁-C₅alkyl; cyclohexyl optionally substituted with one or more C₁-C₅alkyl; phenyl optionally substituted with a heterocyclic radical, aminocarbonyl or C₁-C₅alkylcarboxy;

when n₁ is 2,

R₁₃ is an alkylene-, cycloalkylene, alkenylene or phenylene radical which is optionally substituted by a carbonyl- or carboxy group; a radical of formula *—CH₂—C≡C—CH₂—* or R₁₃ together with A₂ forms a bivalent radical of the formula

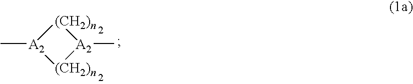

wherein

n₂ is a number from 1 to 3;

when n₁ is 3,

R₁₃ is an alkantriyl radical;

when n₁ is 4,

R₁₃ is an alkantetrayl radical;

A₂ is —O—; or —N(R₁₅)—; and

R₁₅ is hydrogen; C₁-C₅alkyl; or hydroxy-C₁-C₅alkyl.

Most preferred in the method of the present invention is the micronized insoluble UV absorber of the formula

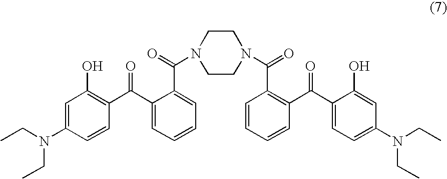

wherein

C₁-C₂₀alkyl denotes a linear or branched, unsubstituted or substituted alkyl group such as, for example, methyl, ethyl, propyl, isopropyl, n-butyl, n-hexyl, cyclohexyl, n-decyl, n-dodecyl, n-octadecyl, eicosyl, methoxyethyl, ethoxypropyl, 2-ethylhexyl, hydroxyethyl, chloropropyl, N,N-diethylaminopropyl, cyanoethyl, phenethyl, benzyl, p-tert-butylphenethyl, p-tert-octylphenoxyethyl, 3-(2,4-di-tert-amylphenoxy)-propyl, ethoxycarbonylmethyl-2-(2-hydroxyethoxy)ethyl or 2-furylethyl.

C₂-C₂₀alkenyl is for example allyl, methallyl, isopropenyl, 2-butenyl, 3-butenyl, isobutenyl, n-penta-2,4-dienyl, 3-methyl-but-2-enyl, n-oct-2-enyl, n-dodec-2-enyl, iso-dodecenyl, n-dodec-2-enyl or n-octadec-4-enyl.

C₃-C₁₀cycloalkyl is for example cyclopropyl, cyclobutyl, cyclopentyl, cycloheptyl, cyclooctyl, cyclononyl or cyclodecyl and preferably cyclohexyl. These radicals may be substituted, for example by one or more oder equal or different C₁-C₄alkyl radicals, preferably by methyl, and/or hydroxy. If cycloalkyl radicals are substituted by one or more radicals, they are preferably substituted by one, two or four, preferably by one or two equal or radicals.

C₃-C₁₀cycloalkenyl is for example cyclopropenyl, cyclobutenyl, cyclopentenyl, cycloheptenyl, cycloocentyl, cyclononenyl or cyclodecenyl and preferably cyclohexenyl. These radicals may be substituted with one or more equal or different C₁-C₄alkyl radical, preferably with methyl, and/or hydroxy. If cycloalkenyl radicals are substituted with one or more radicals they are preferably substituted with one, two, three or four, preferably with one or two equal or different radicals.

Hydroxy-substituted C₁-C₅alkyl groups are for example hydroxymethyl, hydroxyethyl, hydroxypropyl, hydroxybutyl or hydroxypentyl.

An alklyene radical is preferably a C₁-C₁₂alkylene radical, like for example methylene, ethylene, propylene, butylene, hexylene or octylene.

The alklyene radicals may optionally be substituted by one or more C₁-C₅alkyl radicals.

If R₁ and R₂ are heterocyclic radicals, these comprise one, two, three or four equal or different ring hetero atoms. Special preference is given to heterocycles which contain one, two or three, especially one or two, identical or different hetero atoms. The heterocycles may be mono- or poly-cyclic, for example mono-, bi- or tri-cyclic. They are preferably mono- or bi-cyclic, especially monocyclic. The rings preferably contain 5, 6 or 7 ring members. Examples of monocyclic and bicyclic heterocyclic systems from which radicals occurring in the compounds of formula (1) or (2) may be derived are, for example, pyrrole, furan, thiophene, imidazole, pyrazole, 1,2,3-triazole, 1,2,4-triazole, pyridine, pyridazine, pyrimidine, pyrazine, pyran, thiopyran, 1,4-dioxane, 1,2-oxazine, 1,3-oxazine, 1,4-oxazine, indole, benzothiophene, benzofuran, pyrrolidine, piperidine, piperazine, morpholine and thiomorpholine.

The sparingly soluble organic compounds which are used in the present invention are present in the micronized state and are preferably prepared by wet-milling processes.

As milling apparatus for the preparation of the sparingly soluble micronised organic compounds there may be used, for example, a jet mill, ball mill, vibratory mill or hammer mill, preferably a high-speed mixing mill. Even more preferable mills are modern ball mills; manufacturers of these types of mill are, for example, Netzsch (LMZ mill), Drais (DCP-Viscoflow or Cosmo), Bühler AG (centrifugal mills) or Bachhofer.

The insoluble organic UV absorbers used in the present invention are preferably micronized in the presence of a grinding aid.

Preferred useful grinding aids are any surface active ingredients that can be used as dispersing agents. Such surface active ingredient may comprise an anionic, a non ionic, an amphoteric or/and a cationic surfactant, or mixture thereof.

Preferably the grinding aid is used in a concentration of 0.1 to 20% by weight, preferably 1 to 15% b.w. based on the total weight of the UV protection composition.

Preferred grinding aids are carboxylic acids and their salts (b₁), for example
organic basis soap such as linear C₆-C₂₄ fatty acids (capric/caprylic, myristic, palmitoleic, isostearic, linoleic, arachidic, behenic, erucic acids) or branched carboxylic acids or hydroxycarboxylic acids;

Most preferred is Sodium Lauroyl Lactylate.

Further preferred grinding aids are fatty acid esters (b₂), for example
esters of linear C₃-C₇ or C₂₃-C₂₄fatty alcohols with linear fatty acids having from 6 to 11 carbon atoms or more than 22 carbon atoms in the alkyl group

Most preferred is isocetyl isostearate or glycol oleate.

Further preferred grinding aids (b₃) are alkyl phosphates or phosphoric acid esters; such as DEA-oleth-3 phosphate.

Further preferred grinding aids (b₄) are ethoxylated carboxylic acids or polyethyleneglycol (PEG) esters such as PEG-n Acylates, except PEG-n Stearate, PEG-n Oleate, PEG-n

Cocoate, in which the carboxylic acids have alkyl group, ethoxylated or not, with 8 to 22 carbon atoms such as butyric, caproic, caprylic, capric, lauric, myristic, myristoleic, palmitic, palmitoleic, linoleic, arachidic, arachidonic, behenic, eicosapentanoic, erucic or docosahexanoic.

Most preferred is PEG-20 laurate.

Further preferred grinding aids (b₅) are fatty alcohol polyethyleneglycol (PEG) ethers of fatty alcohols.

Where linear or branched fatty alcohols having from 8 to 22 carbon atoms (isopropyl, ethylhexyl, capryl/caprylyl, isotridecyl, myristyl, palmoleyl, isostearyl, linoyl, linolenyl, arachidyl, behenyl or erucyl.

Most preferred is ceteth-10, Laureth-7 or PEG-10 behenyl ether (Beheneth-10).

Further preferred grinding aids (b₇) are ethoxylated alkylphenols or ethoxylated alkylphenyl ethers such as PEG-10 nonyl phenyl ether.

Further preferred grinding aids (b₈) are esters of polyol and mono-, di- or tri-glycerides such as PEG-10 polyglyceryl-2 laurate.

Further preferred grinding aids (b₉) are esters of fatty acids and saccharose such as PEG-120 methyl glucose dioleate or polyglyceryl-3 methylglucose distearate.

Further preferred grinding aids (b₁₀) are sorbitan mono- and di-esters of saturated and/or unsaturated fatty acids such as PEG-20 sorbitan Isostearate and polysorbate-80.

Further preferred grinding aids (b₁₁) are surfactants which are generally acting as detergent or cleansing agents. Examples are listed below:

### A. Anionic Surfactants

Anionic surfactants are designated as such due to the presence of a negatively charged fatty moiety. Such ionised moiety can be a carboxylate, a sulfate, a sulfonate or a phosphate.

General form of anionic surfactant is

R X⁻ M⁺, wherein

R defines the carbon chain length;

X: is negatively charged species such as carboxylate (RCOO⁻), sulfate (ROSO₂O⁻), sulfonate (RSO₂O⁻), or phosphate (ROPO(OH)O⁻)

M is a neutralizing group such as sodium, ammonium, TEA or magnesium.

a. Sulfates; Sulfuric Acids and Salts

Most preferred is Sodium dicocoylethylene diamine PEG-15 sulfate.

b. Sulfonates; Sulfonic Acids and Salts

Acyl Isethionates salts such as sodium acyllsethionate, sodium Cocoyl Isethionate, alkylaryl sulfonates salts such as sodium alkylbenzene sulfonate, sodium dodecylbenzene sulfonate; alkyl Sulfonates salts such as sodium alkylether sulfonate (sodium C12-15 pareth-15 sulfonate); Sodium C₁₄-C₁₆ olefin sulfonate, Sodium decylglucosides Hydroxypropyl sulfonate, or Sodium Laurylglucosides Hydroxypropyl sulfonate

Most preferred is hydroxypropyl sulfonate.

c. Sulfosuccinates; Sulfosuccinic Acids and Salts

Most preferred is disodium alkylamido PEG-n sulfosuccinate (Disodium oleamido MEA-sulfosuccinate).

d. Phosphates; Phosphoric Acids and Salts

PEG-n alkyl phosphates such as DEA oleth-10 phosphate, di PEG-n alkyl phosphates (di-esters) such as dilaureth-4 phosphate.

e. Acylamino Acid and Salts

Acyl glutamates such as Di-TEA palmitoyl aspartate, sodium hydrogenated tallow glutamate; Sodium stearoyl glutamate; acyl peptides such as palmitoyl hydrolysed milk protein, sodium cocoyl hydrolysed soy protein, TEA-cocoyl hydrolysed collagen; Sarcosinates or acyl sarcosides such as myristoyl sarcosine, Sodium lauroyl sarcosinate, sodium myristoyl sarcosinate TEA-lauroyl sarcosinate; taurates and sodium methyl acyltaurates such as sodium lauroyl taurate or sodium methyl cocoyl taurate.

### B. Non Ionic Surfactants

Amine Oxides

Examples are cocamidopropyl amine oxide or lauramine oxide.

### C. Amphoteric or Zwiteterionic Surfactants

Surfactants that carry a positive charge in strongly acidic media, carry anegative charge in strongly basic media and form zwitterionic species at intermediate pH.

a. Acyl/Dialkyl Ethylenediamines

Examples are disodium acylamphodipropionate, sodium acylamphohydroxypropylsulfonate, disodium acylamphodiacetate, sodium acylamphopropionate.

### D. Cationic Surfactants

Surfactants that carry a positive charge; major interesting hair care for conditioning and anti-static effects.

a. Alkylamines

Such as dimethyl alkylamine (dimethyl lauramine), dihydroxyethyl alkylamine dioleate, Acylamidopropyldimethylamine lactate (cocamidopropyl dimethylamine lactate)

b. Alkyl Imidazolines

Such as alkyl hydroxyethyl imidazoline, Ethylhydroxymethyl oleyl oxazoline, alkyl aminoethyl imidazoline

c. Quaternary Compounds

Examples are dialkyldimonium chloride (hydroxyethyl cetyldimonium chloride), alkylamidopropyl alkyldimonium tosylate (Cocamidopropyl ethyldimonium ethosulfate

Further preferred grinding aids (b₁₂) are silicones or organosubstituted polysiloxanes, i.e. any organosilicon polymers or oligomers having a linear or cyclic, branched or crosslinked structure, of variable molecular weight, and essentially based of recurring structural units in which the silicone atoms are linked to each other by oxygen atoms (siloxane bond SiOSi), optionally substituted hydrocarbon radicals being directly linked via a carbon atom to the silicone atoms.

### D. Silicium Containing Compounds

a. Silanol Compounds or Dimethiconols

Dimethyl siloxane terminated with hydroxyl groups (—OH) of the general formula

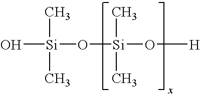

b. Silicone Elastomers & Resins

Crosslinking of siloxane structures such as Dimethicones. Elastomer: medium crosslinking with a density that allows elongation/distorsion of the molecule. We have to exclude PEG-modified Dimethicone Crosspolymers. Resin: high crosslinking with a density that provides some rigidity to the molecule

c. Silicone Elastomers as Co-emulsifier Systems

Dimethicone Crosspolymer in Cyclopentasiloxane; DC 9045 silicone elastomer blend (Dow Corning);Dimethicone Crosspolymer in Dimethicone; DC 9041 silicone elastomer blend (Dow Corning); polymer of Dimethicone (q.v.) crosslinked with a C3 to C20 alkyl group Dimethicone/Vinyldimethicone Crosspolymer; DC 9506 powder (Dow Corning) ; Dimethicone/Vinyldimethicone Crosspolymer in Cyclopentasiloxane; SFE 839 (GE silicones) or KSG 15(Shin-Etsu); copolymer of dimethylpolysiloxane crosslinked with vinyl dimethylpolysiloxane.

d. Resin Silicones

Examples are dispersing agents such as KP-545 (Shin-Etsu); acrylates/dimethicone copolymer in cyclopentasiloxane; copolymer of dimethicone and one or more monomers of acrylic acid, methacrylic acid or one of their simple esters; Siloxysilicates such as Trimethylsiloxysilicates ; T-resins; branched polymer of T-Units; Q-resins; branched polymer of Q-Units:

### [(CH₃)₃SiO_{1/2}]ₓ [SiO₂]_{y}

Film-forming and water-resistant agents such as Trimethylsiloxysilicate; SR 399 (GE Silicones) or Wacker-Belsil TMS803 (Wacker Chemie); mixtures from Dow Corning such as DC 749 cosmetic fluid (Trimethylsiloxysilicate in Cyclopentasiloxane) or DC 593 fluid (Trimethylsiloxysilicate in Dimethicone); Alkyl-Modified Siloxanes (AMS); AMS improve spreadability and wash-off resistance.

For inorganic sunscreens, it improves particle dispersion, reduce the re-agglomeration and better long-lasting effect on skin.

Alkyl Dimethicone of the general formula

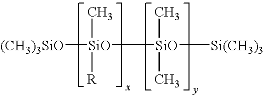

wherein

R is —(CH2)ₙ—CH₃

For example: Bis-Phenylpropyl Dimethicone (SF 1555 fluid; GE Silicone)

Alkyl Methicone of the general formula

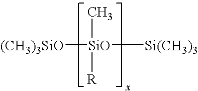

wherein

R is —(CH2)ₙ—CH₃.̂

Silicone waxes such as DC 2503 cosmetic wax (Dow Corning); Stearyl Dimethicone; DC 2502 fluid (Dow Corning); Cetyl Dimethicone; DC AMS-C30 wax (Dow Corning); C30-C45 Alkyl Methicone; DC 580 wax (Dow Corning); Stearoxytrimethylsilane and Stearyl Alcohol

Also suitable are simethicones, which are mixtures of dimethicones having an average chain length of from 200 to 300 dimethylsiloxane units with hydrogenated silicates. A detailed survey by Todd et al. of suitable volatile silicones may in addition be found in Cosm. Toil. 91, 27 (1976).

Silicone emulsifiers particularly suitable for such kind of emulsions are those corresponding to the following formula

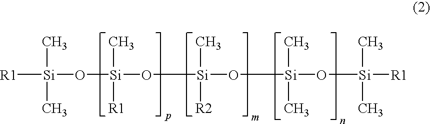

wherein

m is a number from 1 to 20;

n is a umber from 0 to 500; and

p is a number from 0 to 50;

R₁ is linear or branched C₁-C₃₀ alkyl radical or phenyl radical;

R₂ is —C_{c}H_{2c}(—O—C₂H₄)ₐ—(—O—C₃H₆)_{b}—(—O—C₄H₈)_{d}—R₃;

R₃ is H, —OH; linear or branched C₁-C₁₂alkyl, linear or branched C₁-C₆alkoxy, or linear or branched C₂-C₁₂acyloxy; —NHCH₂CH₂COOM; aminoalkyl radical optionally substituted on the amine function; —NHCO(CH₂)_{d}—COOM, C₁-C₃₀carboxyacyl radical;

M is H; Na; K; Li; NH₄; or organic amine; optionally substituted phosphono group; —NHCO(CH₂)_{d} OH; NH₃Y;

Y is a monovalent organic or inorganic anion such as Cl, Br, Sulfate, Carboxylate (Acetate, Lactate, Citrate);

a is a number from 0 to 100;

c is a number from 0 to 5;

b is a number from 0 to 50; and

d is a number from 0 to 10.

These compounds represent the oxyalkylenated organo-modified silicones. Other nomenclature used is PEG/PPG Dimethicones (Dimethicone copolyols) or Silicone polyethers which clearly show surface active properties necessary to emulsify.

Preferred silicone emulsifiers which are particularly recommended correspond to formula

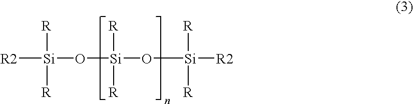

wherein

R is linear or branched C₁-C₃₀ alkyl radical or phenyl radical;

R₂ is —C_{c}H2_{c}—(—O—C₂H₄)ₐ—(—O—C₃H₆)_{b}—O(—C₄H₈)_{d}—R₃;

n is a number from1 to 500; and

R₃, a, b, c and d have the same meaning as described above

Most preferred is dimethicone PEG/PPG—7/4 phosphate.

Rheology modifiers (component (c)) are optionally added to the UV protection composition which help to stabilize across the time such composition.

Examples for rheology modifiers are synthetic polymers, natural polymers and their derivatives, mineral polymers etc., but also according to their ionic character such as anionic, cationic, nonionic or amphoteric as listed in the Table below:

TABLE 1a
Natural thickeners

| | |
|---|---|
| RM 1 | Cellulose gum such as cross-linked or not Sodium Carboxymethylcellulose . . . or |
| | even Cocodimonium Hydroxypropyloxyethyl Cellulose |
| RM 2 | Microcrystalline cellulose and Carboxymethyl Cellulose Sodium |
| RM 3 | Guar gum and derivatives (except hydroxypropyl-modified), -Biosacccharide |
| | gum-1 (Fucogel 1000 from Solabia), -Sclerotium Gum (Amigel from Alban |
| | Muller) or Scleroglucan (Tinocare GL from Ciba SC) |
| RM 4 | Galactoarabinan from Larch extract (Laracare A200) |
| RM 5 | Acaccia/Arabic Gum |
| RM 6 | Konjac mannan; linear chains of glucose and mannose units linked in (β-1,4) |
| RM 7 | Pectin polysaccharides; backbone of galacturonic acid and rhamnose with side |
| | chains as Rhamnogalacturonan I or Rhamnogalacturonan II |
| RM 8 | Xanthan Gum; (β-1,4) linked Glucose residues or Dehydroxanthan Gum |
| | (Amaze XT from National Starch) |
| RM 9 | Starch and derivatives: Potato starch modified (Structure Solanace from |
| | National Starch); Hydroxypropyl Starch Phosphate (Structure XL or ZEA from |
| | National Starch); Amylose and Amylopectin polymeric forms; Maltodextrins |
| RM 10 | Carrageenan from red algae as Sulfated linear polysaccharides |
| RM 11 | Alginic acid and alginates from brown algae; polymers of mannuronic acid and |
| | Guluronic acid |

Most of them are derived from the Polysaccharides category

TABLE 2b
Mineral thickeners

| | |
|---|---|
| RM 12 | Aluminum Silicates or Bentonites or Montmorillonites such as Magnesium |
| | Aluminum Silicates (Veegum range from R. T. Vanderbilt) and Quaternized |
| | compounds such as Stearalkonium Bentonite |
| RM 13 | Magnesium Silicates or Hectorites such as Bentone Series (from Elementis |
| | Specialties) and Quaternized compounds such as Disteardimonium Hectorite |
| | (to disperse in lipophilic media) |
| RM 14 | Magnesium sodium Fluorosilicate or modified Mica |
| RM 15 | Synthetic layered Silicates; similar structure to Hectorites; Sodium Magnesium |
| | Silicates (Laponite range from Solvay) |
| RM 16 | Fumed Silicas such as Aerosil range from Degussa |

Most of them are derived from smectite clays and silica derivatives

TABLE 2c
Synthetic Rheology modifiers

| | |
|---|---|
| RM 17 | Carbomer or crosslinked polyacrylic acid polymer such as Carbopol Ultrez 10, |
| | Carbopol ETD2001, Carbopol ETD2050 from Noveon Inc |
| RM 18 | Sodium polyacrylate (Cosmedia SP from Cognis), Acrylates copolymer |
| | (Carbopol Aqua SF-1 from Noveon Inc.), Acrylates/acrylamides Coplymer |
| | (Noveon EC-1 from Noveon Inc.) |
| RM 19 | Hydroxyethyl/Acrylate/Sodium Acryloyldimethyl Taurate copolymer (Simulgel |
| | NS or EG from Seppic); combination with Tinosorb M claimed in PCA N°161 |
| | November 2001 |
| RM 20 | Ammonium Polyacrylates (Simulgel A from Seppic) |

=>“Hydro Swelling Droplets” concept

| | |
|---|---|
| RM 21 | - Glyceryl Polyacrylates (e.g., Hispagel 100) or Polymethacrylates (e.g., |
| | Lubrajel range from ISP Corp.) |
| RM 22 | Poly(Acrylamide) PAAm and its copolymers; copolymers of ammonium acrylate |
| | and acrylamide; copolymers of AAam with long hydrophobic chain and |
| | acrylates |
| RM 23 | Poly(Ethylene oxide) PEO and Poly (Propylene oxide) PPO and their |
| | copolymers; these are block terpolymers of EO and PO with the structure ABA |
| | or BAB; A: PEO with good water solubility B: PPO with limited water solubility |
| RM 24 | Poly(VinylPyrrolidone)PVP homopoplymers or Poly(VinylPyrrolidone)/Vinyl |
| | Acetate coplymers |
| RM 25 | Poly (vinylalcohol) PVA |
| RM 26 | VA/Crotonates copolymer Poly(vinylacetate)/Crotonic acid or |
| | VA/Crotonates/Vinyl Neodecanoate copolymer |
| RM 27 | Ethylene/VinylAcetate copolymer such as A.C. coplymer400 (Allied-Signal) |
| RM 28 | PVM/MA copolymers and their esterified derivatives such Ethyl, Isopropyl or |
| | Butyl esters |
| RM 29 | PVM/MA Decadiene Crosspolymer; copolymer of methyl vinyl ether/Maleic |
| | Anhydric (PVM/MA) crosslinked with 1,9-decadiene |
| RM 30 | Polyethylene resins such as PEG-2M to PEG-9M (RITA Corp.) |
| RM 31 | polysiloxanes and copolymers; copolymers of polysiloxanes and other blocks |
| | such as PEO blocks |
| RM 32 | PEG-modified materials, the most commonly used class of non ionic |
| | thickeners with the following basic structure: R(OCH₂CH₂)ₙOH, werein R is |
| | the fatty moiety, like fatty alcohol, glyceryl ester, propylene glycol ester or |
| | carboxylic acid; for example; PEG-150 Distearate; these thickeners are not |
| | susceptible to hydrolysis and offer better viscosity stability under a broad range |
| | of pH and temperature profiles |
| RM 33 | Trihydroxystearin or Glycol Tri-(12-Hydroxystearate) |
| RM 34 | Glyceryl Tribehenate such as Syncrowax HRS-C from Croda |

Poly(acrylic acid) PAA and its copolymers; within such structure, it can be incorporated ester groups, with hydrophilic character such as 2-Hydroxyethyl Methacrylate etc.

TABLE 2d
Phospholipid derivatives

| | |
|---|---|
| RM 35 | 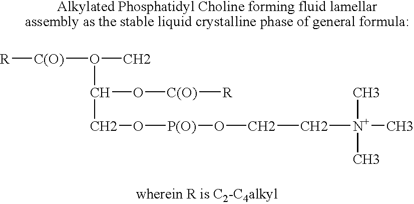 |
| RM 36 | 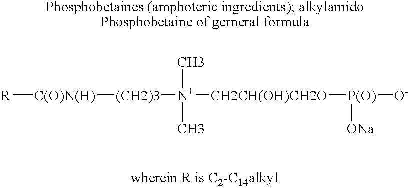 |
| RM 37 | 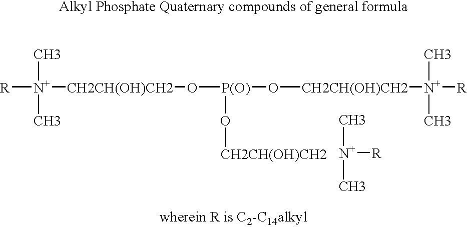 |

Most preferred rheology modifier (c) is Xanthan Gum, amorphous Silica or modified Starch.

Any known process suitable for the preparation of microparticles can be used for the preparation of the micronised UV absorbers, for example:
wet-milling (low-viscosity micronisation process for pumpable dispersions), with a hard grinding medium, for example zirconium silicate balls in a ball mill, and a protective surfactant or a protective polymer in water or in a suitable organic solvent;
wet-kneading (high-viscosity micronisation process for non-pumpable pastes) using a continuous or discontinuous (batch) kneader. For a wet-kneading process, a solvent (water or cosmetically acceptable oils), a grinding aid (surfactant, emulsifier) and a polymeric grinding aid may be used.

Both processes may be used preferably.
spray-drying from a suitable solvent, for example aqueous suspensions or suspensions containing organic solvents, or true solutions in water, ethanol, dichloroethane, toluene or N-methylpyrrolidone etc.
by expansion according to the RESS process (Rapid Expansion of Supercritical Solutions) of supercritical fluids (e.g. CO₂) in which the UV filter or filters is/are dissolved, or the expansion of liquid carbon dioxide together with a solution of one or more UV filters in a suitable organic solvent;
by reprecipitation from suitable solvents, including supercritical fluids (GASR process=Gas Anti-Solvent Recrystallisation/PCA process=Precipitation with Compressed Anti-solvents).

As milling apparatus for the preparation of the micronised organic UV absorbers there may be used, for example, a jet mill, ball mill, vibratory mill or hammer mill, preferably a high-speed mixing mill. Even more preferable mills are modern ball mills; manufacturers of these types of mill are, for example, Netzsch (LMZ mill), Drais (DCP-Viscoflow or Cosmo), Bühler AG (centrifugal mills) or Bachhofer. The grinding is preferably carried out with a grinding aid.

Examples of kneading apparatus for the preparation of the micronised organic UV absorbers are typical sigma-blade batch kneaders but also serial batch kneaders (IKA-Werke) or continuous kneaders (Continua from Werner and Pfleiderer).

PREFERRED EXAMPLES OF PREPARATION OF MICRONIZED UV PROTECTION DISPERSIONS

For each example of micronized UV protection dispersion, the manufacturing process is operated as following;

Compound of formula (7), (5) or (3) respectively are milled together with zirconium silicate bells (diameter 0.1 to 4 mm) as grinding aids, the dispersing agent (as described inside examples A1 to A19) and the continuous phase, containing water, polyol and preservative system, in a ball mill (as described previously) to a mean particle size of d₅₀ from 100 nm to 170 nm. After the micropigment dispersion of UV absorber of formula (7), (5) or (3) respectively is obtained, the formulator incorporate the thickening agent according to the concentration mentioned in examples A1 to A19.

### EXAMPLE A1: Dispersion 1

| | A | B | C | D | E | F | G | H | I |
|---|---|---|---|---|---|---|---|---|---|
| | % | % | % | % | % | % | % | % | % |
| | w/w | w/w | w/w | w/w | w/w | w/w | w/w | w/w | w/w |
| Sparingly soluble micronized substance | | | | | | | | | |
| Compound of formula (7) | 35 | 50 | 65 | | | | | | |
| Compound of formula (5) | | | | 35 | 50 | 65 | | | |
| Compound of formula (3) | | | | | | | 35 | 50 | 65 |
| Dispersing agent | | | | | | | | | |
| Sodium Lauroyl Lactylate | 5 | 7.5 | 10 | 5 | 7.5 | 10 | 5 | 7.5 | 10 |
| Thickening agent | | | | | | | | | |
| modified Starch | | | | 0.4 | 0.3 | 0.2 | | | |
| xanthan gum | 0.2 | 0.2 | 0.3 | | | | 0.2 | 0.3 | 0.15 |
| Continuous phase | | | | | | | | | |
| propylene glycol | 0.3 | 0.3 | 0.3 | 0.3 | 0.3 | 0.3 | 0.3 | 0.3 | 0.3 |
| water | qs. 100 | qs. 100 | qs. 100 | qs. 100 | qs. 100 | qs. 100 | qs. 100 | qs. 100 | qs. 100 |
| Preservative system such as | | | | 0.7 | 0.5 | 0.4 | | | 0.6 |
| Diazolidinyl Urea and parabens | | | | | | | | | |
| Preservative system such as | 0.5 | | 0.6 | | | | 0.4 | | |
| phenoxyethanol and parabens | | | | | | | | | |

### EXAMPLE A2: Dispersion 2

| | A | B | C | D | E | F | G | H | I |
|---|---|---|---|---|---|---|---|---|---|
| | % | % | % | % | % | % | % | % | % |
| | w/w | w/w | w/w | w/w | w/w | w/w | w/w | w/w | w/w |
| Sparingly soluble micronized substance | | | | | | | | | |
| Compound of formula (7) | 50 | | | 50 | | | 40 | | |
| Compound of formula (5) | | 50 | | | 50 | | | 40 | |
| Compound of formula (3) | | | 50 | | | 50 | | | 40 |
| Dispersing agent | | | | | | | | | |
| Isocetyl isostearate | 5 | 5 | 5 | | | | 5 | 5 | |
| Glycol oleate | | | | 5 | 5 | 5 | | | 5 |
| Thickening agent | | | | | | | | | |
| xanthan gum | 0.2 | 0.2 | 0.15 | 0.2 | 0.3 | 0.15 | | | |
| amorphous Silica | | | | | | | 0.3 | | |
| modified Starch | | | | | | | | 0.3 | 0.4 |
| Continuous phase | | | | | | | | | |
| propylene glycol | 0.3 | 0.3 | 0.3 | 0.3 | 0.3 | 0.3 | 0.3 | 0.3 | 0.3 |
| water | qs. 100 | qs. 100 | qs. 100 | qs. 100 | qs. 100 | qs. 100 | qs. 100 | qs. 100 | qs. 100 |
| Preservative system such as | | | | 0.7 | 0.5 | 0.4 | | | 0.6 |
| Diazolidinyl Urea and parabens | | | | | | | | | |
| Preservative system such as | 0.5 | | 0.6 | | | | 0.4 | | |
| phenoxyethanol and parabens | | | | | | | | | |

### EXAMPLE A3: Dispersion 3

| | A | B | C | D | E | F | G | H | I |
|---|---|---|---|---|---|---|---|---|---|
| | % | % | % | % | % | % | % | % | % |
| | w/w | w/w | w/w | w/w | w/w | w/w | w/w | w/w | w/w |
| Sparingly soluble micronized substance | | | | | | | | | |
| Compound of formula (7) | 50 | | | 60 | | | 40 | | |
| Compound of formula (5) | | 50 | | | 60 | | | 40 | |
| Compound of formula (3) | | | 50 | | | 60 | | | 40 |
| Dispersing agent | | | | | | | | | |
| DEA-oleth-3 phosphate | 5 | 5 | 5 | 7 | 7 | 7 | 3 | 3 | 3 |
| Thickening agent | | | | | | | | | |
| xanthan gum | 0.2 | 0.3 | 0.15 | | | | | | |
| amorphous Silica | | | | 0.3 | 0.2 | 0.15 | | | |
| modified Starch | | | | | | | 0.2 | 0.4 | 0.5 |
| Continuous phase | | | | | | | | | |
| propylene glycol | 0.3 | 0.3 | 0.3 | 0.3 | 0.3 | 0.3 | 0.3 | 0.3 | 0.3 |
| water | qs. 100 | qs. 100 | qs. 100 | qs. 100 | qs. 100 | qs. 100 | qs. 100 | qs. 100 | qs. 100 |
| Preservative system such as | | | | 0.7 | 0.5 | 0.4 | | | 0.6 |
| Diazolidinyl Urea and parabens | | | | | | | | | |
| Preservative system such as | 0.5 | | 0.6 | | | | 0.4 | | |
| phenoxyethanol and parabens | | | | | | | | | |

### EXAMPLE A4: Dispersion 4

| | A | B | C | D | E | F | G | H | I |
|---|---|---|---|---|---|---|---|---|---|
| | % | % | % | % | % | % | % | % | % |
| | w/w | w/w | w/w | w/w | w/w | w/w | w/w | w/w | w/w |
| Sparingly soluble micronized substance | | | | | | | | | |
| Compound of formula (7) | 50 | | | 50 | | | 40 | | |
| Compound of formula (5) | | 50 | | | 50 | | | 40 | |
| Compound of formula (3) | | | 50 | | | 50 | | | 40 |
| Dispersing agent | | | | | | | | | |
| PEG-20 Laurate | 6 | 6 | 6 | 4 | 5 | 2.5 | 3 | 8 | 3 |
| Thickening agent | | | | | | | | | |
| xanthan gum | 0.2 | 0.3 | 0.15 | 0.2 | 0.4 | | | 0.2 | |
| amorphous Silica | | | | | | 0.3 | 0.4 | | 0.2 |
| Continuous phase | | | | | | | | | |
| propylene glycol | 0.3 | 0.3 | 0.3 | 0.3 | 0.3 | 0.3 | 0.3 | 0.3 | 0.3 |
| water | qs. 100 | qs. 100 | qs. 100 | qs. 100 | qs. 100 | qs. 100 | qs. 100 | qs. 100 | qs. 100 |
| Preservative system such as | | | | 0.7 | 0.5 | 0.4 | | | 0.6 |
| Diazolidinyl Urea and parabens | | | | | | | | | |
| Preservative system such as | 0.5 | | 0.6 | | | | 0.4 | | |
| phenoxyethanol and parabens | | | | | | | | | |

### EXAMPLE A5: Dispersion 5

| | A | B | C | D | E | F | G | H | I |
|---|---|---|---|---|---|---|---|---|---|
| | % | % | % | % | % | % | % | % | % |
| | w/w | w/w | w/w | w/w | w/w | w/w | w/w | w/w | w/w |
| Sparingly soluble micronized substance | | | | | | | | | |
| Compound of formula (7) | 50 | | | 50 | | | 40 | | |
| Compound of formula (5) | | 50 | | | 50 | | | 40 | |
| Compound of formula (3) | | | 50 | | | 50 | | | 40 |
| Dispersing agent | | | | | | | | | |
| Ceteth-10 | 6 | 6 | 6 | | | | | | |
| PEG-10 Behenyl Ether (Beheneth-10) | | | | 6 | 6 | 6 | | | |
| Laureth-7 | | | | | | | 6 | 6 | 6 |
| Thickening agent | | | | | | | | | |
| xanthan gum | 0.15 | 0.2 | 0.1 | 0.2 | 0.2 | 0.3 | 0.2 | 0.3 | 0.2 |
| Continuous phase | | | | | | | | | |
| propylene glycol | 0.3 | 0.3 | 0.3 | 0.3 | 0.3 | 0.3 | 0.3 | 0.3 | 0.3 |
| water | qs. 100 | qs. 100 | qs. 100 | qs. 100 | qs. 100 | qs. 100 | qs. 100 | qs. 100 | qs. 100 |
| Preservative system such as | | | | 0.7 | 0.5 | 0.4 | | | 0.6 |
| Diazolidinyl Urea and parabens | | | | | | | | | |
| Preservative system such as | 0.5 | | 0.6 | | | | 0.4 | | |
| phenoxyethanol and parabens | | | | | | | | | |

### EXAMPLE A6: Dispersion 6

| | A | B | C | D | E | F | G | H | I |
|---|---|---|---|---|---|---|---|---|---|
| | % | % | % | % | % | % | % | % | % |
| | w/w | w/w | w/w | w/w | w/w | w/w | w/w | w/w | w/w |
| Sparingly soluble micronized substance | | | | | | | | | |
| Compound of formula (7) | 50 | | | 60 | | | 40 | | |
| Compound of formula (5) | | 50 | | | 60 | | | 40 | |
| Compound of formula (3) | | | 50 | | | 60 | | | 40 |
| Dispersing agent | | | | | | | | | |
| Sodium Surfactin | 5 | | 4 | | 3 | | 7 | | 2 |
| Sodium C14-C16 olefin sulfonate | | 5 | | 4 | | 3 | | 7 | 3 |
| Thickening agent | | | | | | | | | |
| xanthan gum | 0.15 | 0.2 | 0.1 | | | | 0.2 | 0.15 | 0.2 |
| modified Starch | | | | 0.3 | 0.2 | 0.4 | | | |
| Continuous phase | | | | | | | | | |
| propylene glycol | 0.3 | 0.3 | 0.3 | 0.3 | 0.3 | 0.3 | 0.3 | 0.3 | 0.3 |
| water | qs. 100 | qs. 100 | qs. 100 | qs. 100 | qs. 100 | qs. 100 | qs. 100 | qs. 100 | qs. 100 |
| Preservative system such as | | | | 0.7 | 0.5 | 0.4 | | | 0.6 |
| Diazolidinyl Urea and parabens | | | | | | | | | |
| Preservative system such as | 0.5 | | 0.6 | | | | 0.4 | | |
| phenoxyethanol and parabens | | | | | | | | | |

### EXAMPLE A7: Dispersion 7

| | A | B | C | D | E | F | G | H | I |
|---|---|---|---|---|---|---|---|---|---|
| | % | % | % | % | % | % | % | % | % |
| | w/w | w/w | w/w | w/w | w/w | w/w | w/w | w/w | w/w |
| Sparingly soluble micronized substance | | | | | | | | | |
| Compound of formula (7) | 50 | | | 60 | | | 40 | | |
| Compound of formula (5) | | 50 | | | 60 | | | 40 | |
| Compound of formula (3) | | | 50 | | | 60 | | | 40 |
| Dispersing agent | | | | | | | | | |
| PEG-10 Nonyl Phenyl ether | 5 | 5 | 5 | 6 | 6 | 6 | 4 | 4 | 4 |
| Thickening agent | | | | | | | | | |
| xanthan gum | 0.15 | 0.2 | 0.1 | | | | 0.2 | 0.15 | 0.2 |
| amorphous Silica | | | | 0.3 | 0.2 | 0.4 | | | |
| Continuous phase | | | | | | | | | |
| propylene glycol | 0.3 | 0.3 | 0.3 | 0.3 | 0.3 | 0.3 | 0.3 | 0.3 | 0.3 |
| water | qs. 100 | qs. 100 | qs. 100 | qs. 100 | qs. 100 | qs. 100 | qs. 100 | qs. 100 | qs. 100 |
| Preservative system such as | | | | 0.7 | 0.5 | 0.4 | | | 0.6 |
| Diazolidinyl Urea and parabens | | | | | | | | | |
| Preservative system such as | 0.5 | | 0.6 | | | | 0.4 | | |
| phenoxyethanol and parabens | | | | | | | | | |

### EXAMPLE A8: Dispersion 8

| | A | B | C | D | E | F | G | H | I |
|---|---|---|---|---|---|---|---|---|---|
| | % | % | % | % | % | % | % | % | % |
| | w/w | w/w | w/w | w/w | w/w | w/w | w/w | w/w | w/w |
| Sparingly soluble micronized substance | | | | | | | | | |
| Compound of formula (7) | 50 | | | 60 | | | 40 | | |
| Compound of formula (5) | | 50 | | | 60 | | | 40 | |
| Compound of formula (3) | | | 50 | | | 60 | | | 40 |
| Dispersing agent | | | | | | | | | |
| PEG-10 polyglyceryl-2 laurate | 5 | 5 | 5 | 6 | 6 | 6 | 4 | 4 | 4 |
| Thickening agent | | | | | | | | | |
| xanthan gum | 0.15 | 0.2 | 0.1 | | | | 0.2 | 0.15 | 0.2 |
| amorphous Silica | | | | 0.3 | 0.2 | 0.4 | | | |
| Continuous phase | | | | | | | | | |
| propylene glycol | 0.3 | 0.3 | 0.3 | 0.3 | 0.3 | 0.3 | 0.3 | 0.3 | 0.3 |
| water | qs. 100 | qs. 100 | qs. 100 | qs. 100 | qs. 100 | qs. 100 | qs. 100 | qs. 100 | qs. 100 |
| Preservative system such as | | | | 0.7 | 0.5 | 0.4 | | | 0.6 |
| Diazolidinyl Urea and parabens | | | | | | | | | |
| Preservative system such as | 0.5 | | 0.6 | | | | 0.4 | | |
| phenoxyethanol and parabens | | | | | | | | | |

### EXAMPLE A9: Dispersion 9

| | A | B | C | D | E | F | G | H | I |
|---|---|---|---|---|---|---|---|---|---|
| | % | % | % | % | % | % | % | % | % |
| | w/w | w/w | w/w | w/w | w/w | w/w | w/w | w/w | w/w |
| Sparingly soluble micronized substance | | | | | | | | | |
| Compound of formula (7) | 50 | | | 50 | | | 40 | | |
| Compound of formula (5) | | 50 | | | 50 | | | 40 | |
| Compound of formula (3) | | | 50 | | | 50 | | | 40 |
| Dispersing agent | | | | | | | | | |
| PEG-120 Methyl Glucose Dioleate | 5 | 5 | 5 | | | | 2 | 2 | 2 |
| Polyglyceryl-3 Methylglucose Distearate | | | | 5 | 5 | 5 | 2 | 2 | 2 |
| Thickening agent | | | | | | | | | |
| xanthan gum | 0.15 | 0.2 | 0.1 | 0.2 | 0.2 | 0.3 | 0.2 | 0.3 | 0.2 |
| Continuous phase | | | | | | | | | |
| propylene glycol | 0.3 | 0.3 | 0.3 | 0.3 | 0.3 | 0.3 | 0.3 | 0.3 | 0.3 |
| water | qs. 100 | qs. 100 | qs. 100 | qs. 100 | qs. 100 | qs. 100 | qs. 100 | qs. 100 | qs. 100 |
| Preservative system such as | | | | 0.7 | 0.5 | 0.4 | | | 0.6 |
| Diazolidinyl Urea and parabens | | | | | | | | | |
| Preservative system such as | 0.5 | | 0.6 | | | | 0.4 | | |
| phenoxyethanol and parabens | | | | | | | | | |

### EXAMPLE A10: Dispersion 10

| | A | B | C | D | E | F | G | H | I |
|---|---|---|---|---|---|---|---|---|---|
| | % | % | % | % | % | % | % | % | % |
| | w/w | w/w | w/w | w/w | w/w | w/w | w/w | w/w | w/w |
| Sparingly soluble micronized substance | | | | | | | | | |
| Compound of formula (7) | 50 | | | 50 | | | 40 | | |
| Compound of formula (5) | | 50 | | | 50 | | | 40 | |
| Compound of formula (3) | | | 50 | | | 50 | | | 40 |
| Dispersing agent | | | | | | | | | |
| PEG-20 Sorbitan Isostearate | 5 | 5 | 5 | | | | 2 | 2 | 2 |
| Polysorbate-80 | | | | 5 | 5 | 5 | 2 | 2 | 2 |
| Thickening agent | | | | | | | | | |
| xanthan gum | 0.15 | 0.2 | 0.1 | 0.2 | 0.2 | 0.3 | 0.2 | 0.3 | 0.2 |
| Continuous phase | | | | | | | | | |
| propylene glycol | 0.3 | 0.3 | 0.3 | 0.3 | 0.3 | 0.3 | 0.3 | 0.3 | 0.3 |
| water | qs. 100 | qs. 100 | qs. 100 | qs. 100 | qs. 100 | qs. 100 | qs. 100 | qs. 100 | qs. 100 |
| Preservative system such as | | | | 0.7 | 0.5 | 0.4 | | | 0.6 |
| Diazolidinyl Urea and parabens | | | | | | | | | |
| Preservative system such as | 0.5 | | 0.6 | | | | 0.4 | | |
| phenoxyethanol and parabens | | | | | | | | | |

### EXAMPLE A11: Dispersion 11

| | A | B | C | D | E | F | G | H | I |
|---|---|---|---|---|---|---|---|---|---|
| | % | % | % | % | % | % | % | % | % |
| | w/w | w/w | w/w | w/w | w/w | w/w | w/w | w/w | w/w |
| Sparingly soluble micronized substance | | | | | | | | | |
| Compound of formula (7) | 50 | | | 60 | | | 40 | | |
| Compound of formula (5) | | 50 | | | 60 | | | 40 | |
| Compound of formula (3) | | | 50 | | | 60 | | | 40 |
| Dispersing agent | | | | | | | | | |
| Decyl glucoside | 5 | | 4 | 3 | 3 | 3 | 2 | | 4 |
| Sodium lauroyl sarcosinate | | 3 | | 1 | | 2 | 4 | 2 | 2 |
| Thickening agent | | | | | | | | | |
| xanthan gum | 0.2 | 0.2 | 0.2 | 0.2 | 0.2 | 0.2 | 0.2 | 0.2 | 0.2 |
| Continuous phase | | | | | | | | | |
| propylene glycol | 0.3 | 0.3 | 0.3 | 0.3 | 0.3 | 0.3 | 0.3 | 0.3 | 0.3 |
| water | qs. 100 | qs. 100 | qs. 100 | qs. 100 | qs. 100 | qs. 100 | qs. 100 | qs. 100 | qs. 100 |
| Preservative system such as | | | | 0.7 | 0.5 | 0.4 | | | 0.6 |
| Diazolidinyl Urea and parabens | | | | | | | | | |
| Preservative system such as | 0.5 | | 0.6 | | | | 0.4 | | |
| phenoxyethanol and parabens | | | | | | | | | |

### EXAMPLE A12: Dispersion 12

| | A | B | C | D | E | F | G | H | I |
|---|---|---|---|---|---|---|---|---|---|
| | % | % | % | % | % | % | % | % | % |
| | w/w | w/w | w/w | w/w | w/w | w/w | w/w | w/w | w/w |
| Sparingly soluble micronized substance | | | | | | | | | |
| Compound of formula (7) | 50 | | | 50 | | | 50 | | |
| Compound of formula (5) | | 50 | | | 50 | | | 50 | |
| Compound of formula (3) | | | 50 | | | 50 | | | 50 |
| Dispersing agent | | | | | | | | | |
| Sodium dicocoylethylene diamine PEG-15 | 5 | 5 | 5 | 3 | 4 | 2 | | | |
| sulfate and Sodium Lauroyl Lactylate | | | | | | | | | |
| Sodium decylglucosides and | | | | 2 | | 3 | 5 | 5 | 5 |
| Hydroxypropyl sulfonate | | | | | | | | | |
| Thickening agent | | | | | | | | | |
| xanthan gum | 0.2 | 0.2 | 0.2 | 0.2 | 0.2 | 0.2 | 0.2 | 0.2 | 0.2 |
| Continuous phase | | | | | | | | | |
| propylene glycol | 0.3 | 0.3 | 0.3 | 0.3 | 0.3 | 0.3 | 0.3 | 0.3 | 0.3 |
| water | qs. 100 | qs. 100 | qs. 100 | qs. 100 | qs. 100 | qs. 100 | qs. 100 | qs. 100 | qs. 100 |
| Preservative system such as | | | | 0.7 | 0.5 | 0.4 | | | 0.6 |
| Diazolidinyl Urea and parabens | | | | | | | | | |
| Preservative system such as | 0.5 | | 0.6 | | | | 0.4 | | |
| phenoxyethanol and parabens | | | | | | | | | |

### EXAMPLE A13: Dispersion 13

| | A | B | C | D | E | F | G | H | I |
|---|---|---|---|---|---|---|---|---|---|
| | % | % | % | % | % | % | % | % | % |
| | w/w | w/w | w/w | w/w | w/w | w/w | w/w | w/w | w/w |
| Sparingly soluble micronized substance | | | | | | | | | |
| Compound of formula (7) | 50 | | | 50 | | | 50 | | |
| Compound of formula (5) | | 50 | | | 50 | | | 50 | |
| Compound of formula (3) | | | 50 | | | 50 | | | 50 |
| Dispersing agent | | | | | | | | | |
| Sodium Laurylglucosides | 5 | 5 | 5 | | | | | | |
| Hydroxypropyl sulfonate | | | | | | | | | |
| DEA Oleth-3 Phosphate | | | | 5 | 5 | 5 | | | |
| Sodium stearoyl glutamate | | | | | | | 5 | 5 | 5 |
| Thickening agent | | | | | | | | | |
| modified Starch | 0.5 | | | 0.4 | 0.3 | 0.2 | | | 0.15 |
| xanthan gum | | 0.2 | 0.2 | | | | 0.2 | 0.2 | |
| Continuous phase | | | | | | | | | |
| propylene glycol | 0.3 | 0.3 | 0.3 | 0.3 | 0.3 | 0.3 | 0.3 | 0.3 | 0.3 |
| water | qs. 100 | qs. 100 | qs. 100 | qs. 100 | qs. 100 | qs. 100 | qs. 100 | qs. 100 | qs. 100 |
| Preservative system such as | | | | 0.7 | 0.5 | 0.4 | | | 0.6 |
| Diazolidinyl Urea and parabens | | | | | | | | | |
| Preservative system such as | 0.5 | | 0.6 | | | | 0.4 | | |
| phenoxyethanol and parabens | | | | | | | | | |

### EXAMPLE A14: Dispersion 14

| | A | B | C | D | E | F | G | H | I |
|---|---|---|---|---|---|---|---|---|---|
| | % | % | % | % | % | % | % | % | % |
| | w/w | w/w | w/w | w/w | w/w | w/w | w/w | w/w | w/w |
| Sparingly soluble micronized substance | | | | | | | | | |
| Compound of formula (7) | 50 | | | 50 | | | 60 | | |
| Compound of formula (5) | | 50 | | | 50 | | | 60 | |
| Compound of formula (3) | | | 50 | | | 50 | | | 60 |
| Dispersing agent | | | | | | | | | |
| Sodium lauroyl sarcosinate | 5 | 5 | 5 | | | | 8 | | 4 |
| sodium myristoyl sarcosinate | | | | 5 | 5 | 5 | | 8 | 4 |
| Thickening agent | | | | | | | | | |
| amorphous Slica | | | | 0.3 | 0.2 | 0.1 | | 0.2 | |
| xanthan gum | 0.2 | 0.2 | 0.2 | | | | 0.2 | | 0.2 |
| Continuous phase | | | | | | | | | |
| propylene glycol | 0.3 | 0.3 | 0.3 | 0.3 | 0.3 | 0.3 | 0.3 | 0.3 | 0.3 |
| water | qs. 100 | qs. 100 | qs. 100 | qs. 100 | qs. 100 | qs. 100 | qs. 100 | qs. 100 | qs. 100 |
| Preservative system such as | | | | 0.7 | 0.5 | 0.4 | | | 0.6 |
| Diazolidinyl Urea and parabens | | | | | | | | | |
| Preservative system such as | 0.5 | | 0.6 | | | | 0.4 | | |
| phenoxyethanol and parabens | | | | | | | | | |

### EXAMPLE A15: Dispersion 15

| | A | B | C | D | E | F | G | H | I |
|---|---|---|---|---|---|---|---|---|---|
| | % | % | % | % | % | % | % | % | % |
| | w/w | w/w | w/w | w/w | w/w | w/w | w/w | w/w | w/w |
| Sparingly soluble micronized substance | | | | | | | | | |
| Compound of formula (7) | 50 | | | 60 | | | 40 | | |
| Compound of formula (5) | | 50 | | | 60 | | | 40 | |
| Compound of formula (3) | | | 50 | | | 60 | | | 40 |
| Dispersing agent | | | | | | | | | |
| Dimethicone PEG/PPG - | 5 | 5 | 5 | 6 | 6 | 6 | 4 | 4 | 4 |
| 7/4 Phosphate | | | | | | | | | |
| Thickening agent | | | | | | | | | |
| xanthan gum | 0.2 | 0.2 | 0.2 | | | | 0.2 | 0.2 | 0.2 |
| amorphous Silica | | | | 0.2 | 0.2 | 0.2 | | | |
| Continuous phase | | | | | | | | | |
| propylene glycol | 0.3 | 0.3 | 0.3 | 0.3 | 0.3 | 0.3 | 0.3 | 0.3 | 0.3 |
| water | qs. 100 | qs. 100 | qs. 100 | qs. 100 | qs. 100 | qs. 100 | qs. 100 | qs. 100 | qs. 100 |
| Preservative system such as | | | | 0.7 | 0.5 | 0.4 | | | 0.6 |
| Diazolidinyl Urea and parabens | | | | | | | | | |
| Preservative system such as | 0.5 | | 0.6 | | | | 0.4 | | |
| phenoxyethanol and parabens | | | | | | | | | |

### EXAMPLE A16: Dispersion 16

| | A | B | C | D | E | F | G | H | I |
|---|---|---|---|---|---|---|---|---|---|
| | % | % | % | % | % | % | % | % | % |
| | w/w | w/w | w/w | w/w | w/w | w/w | w/w | w/w | w/w |
| Sparingly soluble micronized substance | | | | | | | | | |
| Compound of formula (7) | 50 | | | 50 | | | 60 | | |
| Compound of formula (5) | | 50 | | | 50 | | | 60 | |
| Compound of formula (3) | | | 50 | | | 50 | | | 60 |
| Dispersing agent | | | | | | | | | |
| Sodium Laureth Sulfate | 7.5 | 8 | | | 4.5 | | 8 | | 4 |
| Polyglyceryl-10 Laurate | 5 | 4 | | | 5 | 4 | | 5 | |
| Sodium Lauryl Sulfate | | | | | | 7 | | | |
| Sodium Myreth Sulfate | | | 5 | 7 | | | | 8 | 4 |
| Thickening agent | | | | | | | | | |
| Carbomer | | | | 0.3 | 0.2 | 0.1 | | 0.2 | |
| xanthan gum | 0.2 | 0.2 | 0.2 | | | | 0.2 | | 0.2 |
| Continuous phase | | | | | | | | | |
| propylene glycol | 0.3 | 0.3 | 0.3 | 0.3 | 0.3 | 0.3 | 0.3 | 0.3 | 0.3 |
| water | qs. 100 | qs. 100 | qs. 100 | qs. 100 | qs. 100 | qs. 100 | qs. 100 | qs. 100 | qs. 100 |
| Preservative system such as | | | | 0.7 | 0.5 | 0.4 | | | 0.6 |
| Diazolidinyl Urea and parabens | | | | | | | | | |
| Preservative system such as | 0.5 | | 0.6 | | | | 0.4 | | |
| phenoxyethanol and parabens | | | | | | | | | |

### EXAMPLE A17: Dispersion 17

| | A | B | C | D | E | F | G | H | I |
|---|---|---|---|---|---|---|---|---|---|
| | % | % | % | % | % | % | % | % | % |
| | w/w | w/w | w/w | w/w | w/w | w/w | w/w | w/w | w/w |
| Sparingly soluble micronized substance | | | | | | | | | |
| Compound of formula (7) | 50 | | | 50 | | | 60 | | |
| Compound of formula (5) | | 50 | | | 50 | | | 60 | |
| Compound of formula (3) | | | 50 | | | 50 | | | 60 |
| Dispersing agent | | | | | | | | | |
| Decyl Glucoside | 7.5 | 5.5 | | | 4.5 | 4.5 | 8 | | |
| Sodium Myreth Sulfate | 1 | 1.5 | 7.5 | 7 | | | | 7.5 | 4.5 |
| Inulin Lauryl Carbamate | | | 2 | | | 2 | | | 2 |
| Potassium Cetyl Phosphate | | | | | 2.5 | | | 1.5 | |
| Thickening agent | | | | | | | | | |
| Carbomer | | | | 0.3 | 0.2 | 0.1 | | 0.2 | |
| xanthan gum | 0.2 | 0.2 | 0.2 | | | | 0.2 | | 0.2 |
| Continuous phase | | | | | | | | | |
| propylene glycol | 0.3 | 0.3 | 0.3 | 0.3 | 0.3 | 0.3 | 0.3 | 0.3 | 0.3 |
| water | qs. 100 | qs. 100 | qs. 100 | qs. 100 | qs. 100 | qs. 100 | qs. 100 | qs. 100 | qs. 100 |
| Preservative system such as | | | | 0.7 | 0.5 | 0.4 | | | 0.6 |
| Diazolidinyl Urea and parabens | | | | | | | | | |
| Preservative system such as | 0.5 | | 0.6 | | | | 0.4 | | |
| phenoxyethanol and parabens | | | | | | | | | |

### EXAMPLE A18: Dispersion 18

| | A | B | C | D | E | F | G | H | I |
|---|---|---|---|---|---|---|---|---|---|
| | % | % | % | % | % | % | % | % | % |
| | w/w | w/w | w/w | w/w | w/w | w/w | w/w | w/w | w/w |
| Sparingly soluble micronized substance | | | | | | | | | |
| Compound of formula (7) | 50 | | | 50 | | | 60 | | |
| Compound of formula (5) | | 50 | | | 50 | | | 60 | |
| Compound of formula (3) | | | 50 | | | 50 | | | 60 |
| Dispersing agent | | | | | | | | | |
| Sodium C14-17 Sec Alkyl Sulfonate | 8 | | 6 | | 7.5 | | 4 | 6 | |
| Dodecylbenzene Sulfonic Acid | | 6 | | 4.5 | | 7.5 | 5.5 | | 4.5 |
| Sodium Myreth Sulfate | | 1 | | 2 | | 1.5 | | 1 | 2 |
| Thickening agent | | | | | | | | | |
| Carbomer | | | | 0.3 | 0.2 | 0.1 | | 0.2 | |
| xanthan gum | 0.2 | 0.2 | 0.2 | | | | 0.2 | | 0.2 |
| Continuous phase | | | | | | | | | |
| propylene glycol | 0.3 | 0.3 | 0.3 | 0.3 | 0.3 | 0.3 | 0.3 | 0.3 | 0.3 |
| water | qs. 100 | qs. 100 | qs. 100 | qs. 100 | qs. 100 | qs. 100 | qs. 100 | qs. 100 | qs. 100 |
| Preservative system such as | | | | 0.7 | 0.5 | 0.4 | | | 0.6 |
| Diazolidinyl Urea and parabens | | | | | | | | | |
| Preservative system such as | 0.5 | | 0.6 | | | | 0.4 | | |
| phenoxyethanol and parabens | | | | | | | | | |

### EXAMPLE A19: Dispersion 19

| | A | B | C | D | E | F | G | H | I |
|---|---|---|---|---|---|---|---|---|---|
| | % | % | % | % | % | % | % | % | % |
| | w/w | w/w | w/w | w/w | w/w | w/w | w/w | w/w | w/w |
| Sparingly soluble micronized substance | | | | | | | | | |
| Compound of formula (7) | 50 | | | 50 | | | 60 | | |
| Compound of formula (5) | | 50 | | | 50 | | | 60 | |
| Compound of formula (3) | | | 50 | | | 50 | | | 60 |
| Dispersing agent | | | | | | | | | |
| Polyacrylate and Amine/(EO)7-Me | 5 | 8 | 6 | 10 | — | — | — | — | |
| Polyacrylate and Amine/COOH/(EO)7-Me | — | — | — | — | — | 5 | 8 | 6 | 10 |
| Polyacrylate and Amine/(EO)20-Me | — | 1 | 2 | — | 5 | — | — | 2 | — |
| Thickening agent | | | | | | | | | |
| Carbomer | | | | 0.3 | 0.2 | 0.1 | | 0.2 | |
| xanthan gum | 0.2 | 0.2 | 0.2 | | | | 0.2 | | 0.2 |
| Continuous phase | | | | | | | | | |
| propylene glycol | 0.3 | 0.3 | 0.3 | 0.3 | 0.3 | 0.3 | 0.3 | 0.3 | 0.3 |
| water | qs. 100 | qs. 100 | qs. 100 | qs. 100 | qs. 100 | qs. 100 | qs. 100 | qs. 100 | qs. 100 |
| Preservative system such as | | | | 0.7 | 0.5 | 0.4 | | | 0.6 |
| Diazolidinyl Urea and parabens | | | | | | | | | |
| Preservative system such as | 0.5 | | 0.6 | | | | 0.4 | | |
| phenoxyethanol and parabens | | | | | | | | | |

Other preferred preparation of micronized UV protection dispersions are detailed as following:

For each example of micronized UV protection dispersion, the manufacturing process is operated as following:

Compound of formula (7) is milled together with zirconium silicate bells (diameter 0.1 to 4 mm) as grinding aids, the dispersing agent (as described inside dispersions 20 to 34) and the continuous phase, containing water, simethicone and occasionally citric acid, in a ball mill (as described previously) to a mean particle size of d₅₀ from 100 nm to 170 nm. After the micropigment dispersion of UV absorber of formula (7 is obtained, the formulator incorporate the thickening agent (Xanthan Gum) according to the concentration mentioned in dispersions 20 to 34.

### Dispersion 20

| | | | | Particle size | |
|---|---|---|---|---|---|
| | | | Extinction | distribution | |
| | % w/w | pH | coef. E_{1, 1} | d 50 | d 90 |
| Compound of formula | 50 | 7 | 470 | 123 nm | 246 nm |
| (7) | | | | | |
| Sodium dicocoylethylene | 9 | | | | |
| diamine PEG-15 sulfate and | | | | | |
| Sodium Lauroyl Lactylate | | | | | |
| Simethicone | 0.9 | | | | |
| Water | Qs 100 | | | | |
| Xanthan Gum | 0.1 | | | | |

### Dispersion 21

| | | | | Particle size | |
|---|---|---|---|---|---|
| | | | Extinction | distribution | |
| | % w/w | pH | coef. E_{1, 1} | d 50 | d 90 |
| Compound of formula | 45.5 | 8.5 | 860 | 105 nm | 220 nm |
| (7) | | | | | |
| Sodium Lauroyl | 7.5 | | | | |
| Sarcosinate | | | | | |
| Simethicone | 1 | | | | |
| Water | Qs 100 | | | | |
| Xanthan Gum | 0.1 | | | | |

### Dispersion 22

| | | | | Particle size | |
|---|---|---|---|---|---|
| | | | Extinction | distribution | |
| | % w/w | pH | coef. E_{1, 1} | d 50 | d 90 |
| Compound of formula | 50 | 7 | 870 | 123 nm | 238 nm |
| (7) | | | | | |
| Sodium Myristoyl | 7.5 | | | | |
| Sarcosinate | | | | | |
| Simethicone | 1 | | | | |
| Water | Qs 100 | | | | |
| Xanthan Gum | 0.1 | | | | |

### Dispersion 23

| | | | | Particle size | |
|---|---|---|---|---|---|
| | | | Extinction | distribution | |
| | % w/w | pH | coef. E_{1, 1} | d 50 | d 90 |
| Compound of formula | 45 | 7.5 | 755 | 120 nm | 230 nm |
| (7) | | | | | |
| Sodium Stearoyl | 5 | | | | |
| Glutamate | | | | | |
| Simethicone | 1 | | | | |
| Water | Qs 100 | | | | |
| Xanthan Gum | 0.1 | | | | |

### Dispersion 24

| | | | | Particle size | |
|---|---|---|---|---|---|
| | | | Extinction | distribution | |
| | % w/w | pH | coef. E_{1, 1} | d 50 | d 90 |
| Compound of formula | 50 | 3.5 | 650 | 120 nm | 227 nm |
| (7) | | | | | |
| Sodium Laureth sulfate | 7.5 | | | | |
| Simethicone | 1 | | | | |
| Water | Qs 100 | | | | |
| Xanthan Gum | 0.1 | | | | |

### Dispersion 25

| | | | | Particle size | |
|---|---|---|---|---|---|
| | | | Extinction | distribution | |
| | % w/w | pH | coef. E_{1, 1} | d 50 | d 90 |
| Compound of formula | 50 | 11 | 670 | 122 nm | 234 nm |
| (7) | | | | | |
| Sodium C14-17 Sec | 5 | | | | |
| Alkyl Sulfonate | | | | | |
| Simethicone | 1 | | | | |
| Water | Qs 100 | | | | |
| Xanthan Gum | 0.1 | | | | |

### Dispersion 26

| | | | | Particle size | |
|---|---|---|---|---|---|
| | | | Extinction | distribution | |
| | % w/w | pH | coef. E_{1, 1} | d 50 | d 90 |
| Compound of formula | 42.5 | 5 | 650 | 122 nm | 237 nm |
| (7) | | | | | |
| Polyacrylate and | 6.3 | | | | |
| Amine/(EO)20-Me | | | | | |
| Simethicone | 1 | | | | |
| Water | Qs 100 | | | | |
| Citric acid | qs | | | | |
| Xanthan Gum | 0.1 | | | | |

### Dispersion 27

| | | | | Particle size | |
|---|---|---|---|---|---|
| | | | Extinction | distribution | |
| | % w/w | pH | coef. E_{1, 1} | d 50 | d 90 |
| Compound of formula | 45.8 | 3 | 685 | 116 nm | 227 nm |
| (7) | | | | | |
| Sodium Myreth Sulfate | 7.5 | | | | |
| Inulin Lauryl Carbamate | 2 | | | | |
| Simethicone | 1 | | | | |
| Water | Qs 100 | | | | |
| Citric acid | qs | | | | |
| Xanthan Gum | 0.1 | | | | |

### Dispersion 28

| | | | | Particle size | |
|---|---|---|---|---|---|
| | | | Extinction | distribution | |
| | % w/w | pH | coef. E_{1, 1} | d 50 | d 90 |
| Compound of formula | 49 | 3 | 650 | 117 nm | 239 nm |
| (7) | | | | | |
| Sodium Laureth Sulfate | 7.4 | | | | |
| Polyglyceryl-10 Laurate | 4.8 | | | | |
| Simethicone | 1 | | | | |
| Water | Qs 100 | | | | |
| Citric acid | qs | | | | |
| Xanthan Gum | 0.1 | | | | |

### Dispersion 29

| | | | | Particle size | |
|---|---|---|---|---|---|
| | | | Extinction | distribution | |
| | % w/w | pH | coef. E_{1, 1} | d 50 | d 90 |
| Compound of formula | 40.5 | 3 | 480 | 127 nm | 265 nm |
| (7) | | | | | |
| Sodium Laureth Sulfate | 2 | | | | |
| Polyacrylate and | 4.1 | | | | |
| Amine/COOH/(EO)7-Me | | | | | |
| Simethicone | 1 | | | | |
| Water | Qs 100 | | | | |
| Citric acid | qs | | | | |
| Xanthan Gum | 0.1 | | | | |

### Dispersion 30

| | | | | Particle size | |
|---|---|---|---|---|---|
| | | | Extinction | distribution | |
| | % w/w | pH | coef. E_{1, 1} | d 50 | d 90 |
| Compound of formula | 50 | 3 | 644 | 124 nm | 243 nm |
| (7) | | | | | |
| Sodium Laureth Sulfate | 5 | | | | |
| Poloxamer 407 | 2 | | | | |
| Simethicone | 1 | | | | |
| Water | Qs 100 | | | | |
| Citric acid | qs | | | | |
| Xanthan Gum | 0.1 | | | | |

### Dispersion 31

| | | | | Particle size | |
|---|---|---|---|---|---|
| | | | Extinction | distribution | |
| | % w/w | pH | coef. E_{1, 1} | d 50 | d 90 |
| Compound of formula | 45.7 | 11.5 | 610 | 132 nm | 265 nm |
| (7) | | | | | |
| Sodium Myreth Sulfate | 4.6 | | | | |
| Decyl glucoside | 9.1 | | | | |
| Simethicone | 1 | | | | |
| Water | Qs 100 | | | | |
| Xanthan Gum | 0.1 | | | | |

### Dispersion 32

| | | | | Particle size | |
|---|---|---|---|---|---|
| | | | Extinction | distribution | |
| | % w/w | pH | coef. E_{1, 1} | d 50 | d 90 |
| Compound of formula | 40 | 5.9 | 712 | 123 nm | 242 nm |
| (7) | | | | | |
| Sodium Myreth Sulfate | 5 | | | | |
| Polyglyceryl-10 Laurate | 5 | | | | |
| Simethicone | 1 | | | | |
| Water | Qs 100 | | | | |
| Xanthan Gum | 0.1 | | | | |

### Dispersion 33

| | | | | Particle size | |
|---|---|---|---|---|---|
| | | | Extinction | distribution | |
| | % w/w | pH | coef. E_{1, 1} | d 50 | d 90 |
| Compound of formula | 44.3 | 4 | 826 | 115 nm | 226 nm |
| (7) | | | | | |
| Sodium Myreth Sulfate | 1.3 | | | | |
| Dodecylbenzene | 6.2 | | | | |
| Sulfonic Acid | | | | | |
| Simethicone | 1 | | | | |
| Water | Qs 100 | | | | |
| Citric acid | qs | | | | |
| Xanthan Gum | 0.1 | | | | |

### Dispersion 34

| | | | | Particle size | |
|---|---|---|---|---|---|
| | | | Extinction | distribution | |
| | % w/w | pH | coef. E_{1, 1} | d 50 | d 90 |
| Compound of formula | 46.5 | 7 | 752 | 124 nm | 235 nm |
| (7) | | | | | |
| Sodium Myreth Sulfate | 0.9 | | | | |
| PO-EO block copolymer | 4.6 | | | | |
| 40% EO | | | | | |
| Simethicone | 1 | | | | |
| Water | Qs 100 | | | | |
| Xanthan Gum | 0.1 | | | | |

The UV absorber composition preferably comprises
(a) 0.1 to 25% by weight of a micronised insoluble organic UV absorber dispersion according to the present invention; and optionally
(b) a cosmetically acceptable carrier.

The UV absorber composition according to the present invention may comprise one or more than one additional UV absorbers as described in the Tables 3 and 4.

Preference is given to the use of mixing ratios of the compound of formula (1), (2) or (3) according to the present invention and optionally further UV absorbers as described in the Table 1 and 2 from 1:99 to 99:1, preferably from 1:95 to 95:1 and most preferably from 10:90 to 90:10, based on weight.

Of special interest are mixing ratios of from 20:80 to 80:20 and most preferably from 40:60 to 60:40.

TABLE 3
Suitable UV filter substances which can be additionally used
with the UV absorbers according to the present invention

p-aminobenzoic acid derivatives, for example 4-dimethylaminobenzoic acid 2-ethylhexyl
ester;
salicylic acid derivatives, for example salicylic acid 2-ethylhexyl ester;
benzophenone derivatives, for example 2-hydroxy-4-methoxybenzophenone and its
5-sulfonic acid derivative;
diphenylacrylates, for example 2-ethylhexyl 2-cyano-3,3-diphenylacrylate, and 3-(benzo-
furanyl) 2-cyanoacrylate;
3-imidazol-4-ylacrylic acid and esters;
benzofuran derivatives, especially 2-(p-aminophenyl)benzofuran derivatives, described in
EP-A-582 189, U.S. Pat. No. 5,338,539, U.S. Pat. No. 5,518,713 and EP-A-613 893;
polymeric UV absorbers, for example the benzylidene malonate derivatives described in
EP-A-709 080;
cinnamic acid derivatives, for example the 4-methoxycinnamic acid 2-ethylhexyl ester and
isoamyl ester or cinnamic acid derivatives described in U.S. Pat. No. 5,601,811 and WO 97/00851;
camphor derivatives, for example 3-(4′-methyl)benzylidene-bornan-2-one, 3-benzylidene-
bornan-2-one, N-[2(and 4)-2-oxyborn-3-ylidene-methyl)-benzyl]acrylamide polymer, 3-(4′-
trimethylammonium)-benzylidene-bornan-2-one methyl sulfate, 3,3′-(1,4-phenylene-
dimethine)-bis(7,7-dimethyl-2-oxo-bicyclo[2.2.1]heptane-1-methanesulfonic acid) and salts,
3-(4′-sulfo)benzylidene-bornan-2-one and salts; camphorbenzalkonium methosulfate;
hydroxyphenyltriazine compounds, for example 2-(4′-methoxyphenyl)-4,6-bis(2′-hydroxy-4′-n-
octyloxyphenyl)-1,3,5-triazine; 2,4-bis{[4-(3-(2-propyloxy)-2-hydroxy-propyloxy)-2-hydroxy]-
phenyl}-6-(4-methoxyphenyl)-1,3,5-triazine; 2,4-bis{[4-(2-ethyl-hexyloxy)-2-hydroxy]-phenyl}-
6-[4-(2-methoxyethyl-carboxyl)-phenylamino]-1,3,5-triazine; 2,4-bis{[4-(tris(trimethylsilyloxy-
silylpropyloxy)-2-hydroxy]-phenyl}-6-(4-methoxyphenyl)-1,3,5-triazine; 2,4-bis{[4-(2″-
methylpropenyloxy)-2-hydroxy]-phenyl}-6-(4-methoxyphenyl)-1,3,5-triazine; 2,4-bis{[4-
(1′,1′,1′,3′,5′,5′,5′-heptamethyltrisilyl-2″-methyl-propyloxy)-2-hydroxy]-phenyl}-6-(4-
methoxyphenyl)-1,3,5-triazine; 2,4-bis{[4-(3-(2-propyloxy)-2-hydroxy-propyloxy)-2-hydroxy]-
phenyl}-6-[4-ethylcarboxy)-phenylamino]-1,3,5-triazine;
physical sunscreens, coated or not coated, such as titanium dioxide, zinc oxide, iron oxides,
mica, MnO, Fe₂O₃, Ce₂O₃, Al₂O₃, ZrO₂ (surface coatings: polymethylmethacrylate, methicone
(methylhydrogenpolysiloxane as described in CAS 9004-73-3), dimethicone, isopropyl
titanium triisostearate (as described in CAS 61417-49-0), metal soaps such as magnesium
stearate (as described in CAS 4086-70-8), perfluoroalcohol phosphate such as C₉-C₁₅
fluoroalcohol phosphate (as described in CAS 74499-44-8; JP 5-86984; JP 4-330007)). The
primary particle size is, on average, 15 nm-35 nm and the particle size distribution is in the
range 100 nm-300 nm.
aminohydroxy-benzophenone derivatives disclosed in DE 100 11 317, EP 1 133 980 and
EP 1 046 391
phenyl-benzimidazole derivatives as disclosed in EP 1 167 358

TABLE 4
Suitable UV filter substances and adjuvants which can be additionally
used with theUV absorbers according to the
present invention

| No. | Chemical Name | CAS No. |
|---|---|---|
| 1 | (+/−)-1,7,7-trimethyl-3-[(4-methylphenyl)methylene] | 36861-47-9 |
| | bicyclo-[2.2.1]heptan-2-one; p-methyl benzylidene | |
| | camphor | |
| 2 | 1,7,7-trimethy1-3-(phenylmethylene)bicyclo[2.2.1] | 15087-24-8 |
| | heptan-2-one; benzylidene camphor | |
| 3 | (2-Hydroxy-4-methoxyphenyl)(4-methylphenyl) | 1641-17-4 |
| | methanone | |
| 4 | 2,4-dihydroxybenzophenone | 131-56-6 |
| 5 | 2,2′,4,4′-tetrahydroxybenzophenone | 131-55-5 |
| 6 | 2-Hydroxy-4-methoxy benzophenone; | 131-57-7 |
| 7 | 2-Hydroxy-4-methoxy benzophenone-5-sulfonic acid | 4065-45-6 |
| 8 | 2,2′-dihydroxy-4,4′-dimethoxybenzophenone | 131-54-4 |
| 9 | 2,2′-Dihydroxy-4-methoxybenzophenone | 131-53-3 |
| 10 | Alpha-(2-oxoborn-3-ylidene)toluene-4-sulphonic | 56039-58-8 |
| | acid and its salts; Mexoryl SL | |
| 11 | 1-[4-(1,1-dimethylethyl)phenyI]-3-(4-methoxyphenyl) | 70356-09-1 |
| | propane-1,3-dione; avobenzone | |
| 12 | Methyl N,N,N-trimethy1-4-[(4,7,7-trimethyl-3-oxo- | 52793-97-2 |
| | bicyclo[2,2,1]hept-2-ylidene)methyl]anilinium sulphate; | |
| | Mexoryl SO | |
| 22 | 3,3,5-Trimethyl cyclohexyl-2-hydroxy benzoate; | 118-56-9 |
| | homosalate | |
| 23 | Isopentyl p-methoxycinnamate; isoamyl methoxy | 71617-10-2 |
| | cinnamate | |
| 27 | Menthyl-o-aminobenzoate | 134-09-8 |
| 28 | Menthyl salicylate | 89-46-3 |
| 29 | 2-Ethylhexyl 2-cyano,3,3-diphenylacrylate; octocrylene | 6197-30-4 |
| 30 | 2-ethylhexyl 4-(dimethylamino)benzoate | 21245-02-3 |
| 31 | 2-ethylhexyl 4-methoxycinnamate; octyl methoxy | 5466-77-3 |
| | cinnamate | |
| 32 | 2-ethylhexyl salicylate | 118-60-5 |
| 33 | Benzoic acid, 4,4′,4″-(1,3,5-triazine-2,4,6-triyltriimino) | 88122-99-0 |
| | tris(2-ethylhexyl)ester; 2,4,6-Trianilino-(p-carbo-2′- | |
| | ethylhexyl-1′-oxi)-1,3,5-triazine; octyl triazone | |
| 34 | 4-aminobenzoic acid | 150-13-0 |
| 35 | Benzoic acid, 4-amino-, ethyl ester, polymer with | 113010-52-9 |
| | oxirane | |
| 38 | 2-phenyl-1H-benzimidazole-5-sulphonic acid; | 27503-81-7 |
| | phenylbenzimidazolsulfonic acid | |
| 39 | 2-Propenamide, N-[[4-[(4,7,7-trimethyl-3-oxo- | 147897-12-9 |
| | bicyclo[2.2.1]hept-2-ylidene)methyl]phenyl]methyl]-, | |
| | homopolymer | |
| 40 | Triethanolamine salicylate | 2174-16-5 |
| 41 | 3,3′-(1,4-phenylenedimethylene)bis[7,7-dimethyl- | 90457-82-2 |
| | 2-oxo-bicyclo[2.2.1]heptane-1 methanesulfonic acid]; | |
| | Cibafast H | |
| 42 | Titanium dioxide | 13463-67-7 |
| 44 | Zinc oxide | 1314-13-2 |
| 46 | 1H-Benzimidazole-4,6-disulfonic acid, 2,2′-(1,4- | 180898-37-7 |
| | phenylene)bis-, disodium salt | |
| 47 | Benzoic acid, 4,4'-[[6-[[4-[[(1,1- | 154702-15-5 |
| | dimethylethyl)amino]carbonyl]phenyl]amino]1,3,5- | |
| | triazine-2,4-diyl]diimino]bis-, bis(2-ethylhexyl)ester; | |
| | diethylhexyl butamido triazone; Uvasorb HEB | |
| 48 | Phenol, 2-(2H-benzotriazol-2-y1)-4-methyl-6-[2- | 155633-54-8 |
| | methy1-3-[1,3,3,3-tetramethyl-1-[(trimethylsily)oxy] | |
| | disiloxanyl]propyl]-; drometrizole trisiloxane; | |
| | Mexoryl XL | |
| 49 | Dimethicodiethylbenzalmalonate; Polysilicone 15; | 207574-74-1 |
| | Parsol SLX | |
| 50 | Benzenesulfonic acid, 3-(2H-benzotriazol-2-yl)- | 92484-48-5 |
| | 4-hydroxy-5-(1-methylpropyl)-, monosodium salt ; | |
| | Tinogard HS | |
| 51 | Benzoic acid, 2-[4-(diethylamino)-2-hydroxybenzoyl]-, | 302776-68-7 |
| | hexyl ester; Uvinul a plus | |
| 52 | 1-Dodecanaminium, N-[3-[[4-(dimethylamino) | 156679-41-3 |
| | benzoyl]amino]propyl]-N,N-dimethyl-, salt with | |
| | 4-methyl-benzenesulfonic acid | |
| | (1:1); Escalol HP610 | |
| 53 | 1-Propanaminium, N,N,N-trimethyl-3-[(1-oxo-3- | 177190-98-6 |
| | phenyl-2-propenyl)-amino]-, chloride | |
| 54 | 1H-Benzimidazole-4,6-disulfonic acid, 2,2′-(1,4- | 170864-82-1 |
| | phenylene)bis- | |
| 55 | 1,3,5-Triazine, 2,4,6-tris(4-methoxyphenyl)- | 7753-12-0 |
| 56 | 1,3,5-Triazine, 2,4,6-tris[4-[(2-ethylhexyl)oxy] | 208114-14-1 |
| | phenyl]- | |
| 57 | 1-Propanaminium, 3-[[3-[3-(2H-benzotriazol- | 340964-15-0 |
| | 2-y1)-5-(1,1-dimethylethyl)-4-hydroxyphenyl]-1- | |
| | oxopropyl]amino]-N,N-diethyl-N-methyl-, | |
| | methyl sulfate (salt) | |
| 58 | 2-Propenoic acid, 3-(1H-imidazol-4-yl)- | 104-98-3 |
| 59 | Benzoic acid, 2-hydroxy-, [4-(1-methylethyl)phenyl] | 94134-93-7 |
| | methyl ester | |
| 60 | 1,2,3-Propanetriol, 1-(4-aminobenzoate); glyceryl PABA | 136-44-7 |
| 61 | Benzeneacetic acid, 3,4-dimethoxy-a-oxo- | 4732-70-1 |
| 62 | 2-Propenoic acid, 2-cyano-3,3-diphenyl-, ethyl ester | 5232-99-5 |
| 63 | Anthralinic acid, p-menth-3-yl ester | 134-09-8 |
| 64 | 2,2′-bis(1,4-phenylene)-1H-benzimidazole-4,6- | 349580-12-7, |
| | disulphonic acid mono sodium salt or Disodium phenyl | |
| | dibenzimidazole tetrasulfonate or Neoheliopan AP | |
| 65 | 1,3,5-Triazine-2,4,6-triamine, N,N′-bis[4-[5-(1,1- | 288254-16-0 |
| | dimethylpropyl)-2- benzoxazolyl]phenyl]-N″- | |
| | (2-ethylhexyl)-or Uvasorb K2A | |
| 66 | Merocyanine derivatives as described in | |
| | WO 2004006878 and in IPCOM000022279D | |
| 67 | 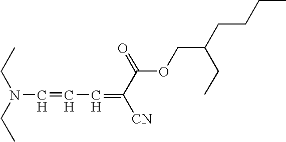 | |
| 68 | sterols (cholesterol, lanosterol, phytosterols), | |
| | as described inWO0341675 | |
| 69 | mycosporines and/or mycosporine-like amino | |
| | acids as described in WO2002039974, e.g. | |
| | Helioguard 365 from Milbelle AG, | |
| | isolated mycosporine like amino acids | |
| | from the red alga porphyra umbilicalis (INCI: | |
| | Porphyra Umbilicalis) that are encapsulated | |
| | into liposomes,) | |
| 70 | alpha-lipoic-acid as described in DE 10229995 | |
| 71 | synthetic organic polymers as described in | |
| | EP 1371358, [0033]-[0041] | |
| 72 | phyllosilicates as described in EP 1371357 [0034]- | |
| | [0037] | |
| 73 | silica compounds as described in EP1371356, | |
| | [0033]-[0041] | |
| 74 | inorganic particles as described in DE10138496 | |
| | [0043]-[0055] | |
| 75 | latex particles as described in DE10138496 [0027]- | |
| | [0040] | |
| 76 | 1H-Benzimidazole-4,6-disulfonic acid, 2,2′-(1,4- | 180898-37-7 |
| | phenylene)bis-, disodium salt; Bisimidazylate; | |
| | Neo Heliopan APC | |
| 77 | 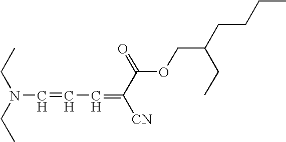 | |
| 78 | 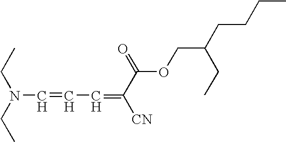 | |
| 79 | 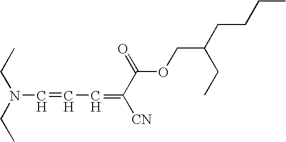 | |
| 80 | 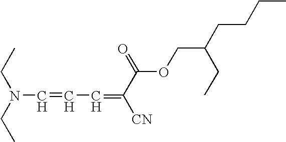 | |
| 83 | Di-2-ethylhexyl-3,5-dimethoxy-4-benzalmalonate | |
| | (Oxynex ST, EMD Chemicals, as described in | |
| | U.S. Pat. No. 20040247536) | |

The cosmetic or pharmaceutical preparations may be, for example, creams, gels, lotions, alcoholic and aqueous/alcoholic solutions, emulsions, wax/fat compositions, stick preparations, powders or ointments. In addition to the above-mentioned UV filters, the cosmetic or pharmaceutical preparations may contain further adjuvants as described below.

As water- and oil-containing emulsions (e.g. W/O, O/W, O/W/O and W/O/W emulsions or microemulsions) the preparations contain, for example, from 0.1 to 30% by weight, preferably from 0.1 to 15% by weight and especially from 0.5 to 10% by weight, based on the total weight of the composition, of one or more UV absorbers, from 1 to 60% by weight, especially from 5 to 50% by weight and preferably from 10 to 35% by weight, based on the total weight of the composition, of at least one oil component, from 0 to 30% by weight, especially from 1 to 30% by weight and preferably from 4 to 20% by weight, based on the total weight of the composition, of at least one emulsifier, from 10 to 90% by weight, especially from 30 to 90% by weight, based on the total weight of the composition, of water, and from 0 to 88.9% by weight, especially from 1 to 50% by weight, of further cosmetically acceptable adjuvants.

The cosmetic or pharmaceutical compositions/preparations according to the invention may also contain one or one more additional compounds like fatty alcohols, esters of fatty acids, natural or synthetic triglycerides including glyceryl esters and derivatives, pearlescent waxes:hydrocarbon oils: silicones or siloxanes, organosubstituted super-fatting agents, surfactantsconsistency regulators/thickeners and rheology modifiers, polymers, biogenic active ingredients, deodorising active ingredients, anti-dandruff agents, antioxidants, hydrotropic agents, preservatives and bacteria-inhibiting agents, perfume oils, colorants, polymeric beads or hollow spheres as spa enhancers.

### Cosmetic or Pharmaceutical Preparations

Cosmetic or pharmaceutical formulations are contained in a wide variety of cosmetic preparations. There come into consideration, for example, especially the following preparations:
skin-care preparations, e.g. skin-washing and cleansing preparations in the form of tablet-form or liquid soaps, soapless detergents or washing pastes,
bath preparations, e.g. liquid (foam baths, milks, shower preparations) or solid bath preparations, e.g. bath cubes and bath salts;
skin-care preparations, e.g. skin emulsions, multi-emulsions or skin oils;
cosmetic personal care preparations, e.g. facial make-up in the form of day creams or powder creams, face powder (loose or pressed), rouge or cream make-up, eye-care preparations, e.g. eyeshadow preparations, mascara, eyeliner, eye creams or eye-fix creams; lip-care preparations, e.g. lipsticks, lip gloss, lip contour pencils, nail-care preparations, such as nail varnish, nail varnish removers, nail hardeners or cuticle removers;
foot-care preparations, e.g. foot baths, foot powders, foot creams or foot balsams, special deodorants and antiperspirants or callus-removing preparations;
light-protective preparations, such as sun milks, lotions, creams or oils, sunblocks or tropicals, pre-tanning preparations or after-sun preparations;
skin-tanning preparations, e.g. self-tanning creams;
depigmenting preparations, e.g. preparations for bleaching the skin or skin-lightening preparations;
insect-repellents, e.g. insect-repellent oils, lotions, sprays or sticks;
deodorants, such as deodorant sprays, pump-action sprays, deodorant gels, sticks or roll-ons;
antiperspirants, e.g. antiperspirant sticks, creams or roll-ons;
preparations for cleansing and caring for blemished skin, e.g. synthetic detergents (solid or liquid), peeling or scrub preparations or peeling masks;
hair-removal preparations in chemical form (depilation), e.g. hair-removing powders, liquid hair-removing preparations, cream- or paste-form hair-removing preparations, hair-removing preparations in gel form or aerosol foams;
shaving preparations, e.g. shaving soap, foaming shaving creams, non-foaming shaving creams, foams and gels, preshave preparations for dry shaving, aftershaves or aftershave lotions;
fragrance preparations, e.g. fragrances (eau de Cologne, eau de toilette, eau de parfum, parfum de toilette, perfume), perfume oils or perfume creams;
cosmetic hair-treatment preparations, e.g. hair-washing preparations in the form of shampoos and conditioners, hair-care preparations, e.g. pretreatment preparations, hair tonics, styling creams, styling gels, pomades, hair rinses, treatment packs, intensive hair treatments, hair-structuring preparations, e.g. hair-waving preparations for permanent waves (hot wave, mild wave, cold wave), hair-straightening preparations, liquid hair-setting preparations, hair foams, hairsprays, bleaching preparations, e.g. hydrogen peroxide solutions, lightening shampoos, bleaching creams, bleaching powders, bleaching pastes or oils, temporary, semi-permanent or permanent hair colourants, preparations containing self-oxidising dyes, or natural hair colourants, such as henna or camomile.

### Presentation Forms

The final formulations listed may exist in a wide variety of presentation forms, for example:
in the form of liquid preparations as a W/O, O/W, O/W/O, W/O/VV or PIT emulsion and all kinds of microemulsions,
in the form of a gel,
in the form of an oil, a cream, milk or lotion,
in the form of a powder, a lacquer, a tablet or make-up,
in the form of a stick,
in the form of a spray (spray with propellent gas or pump-action spray) or an aerosol,
in the form of a foam, or
in the form of a paste.

Of special importance as cosmetic preparations for the skin are light-protective preparations, such as sun milks, lotions, creams, oils, sunblocks or tropicals, pretanning preparations or after-sun preparations, also skin-tanning preparations, for example self-tanning creams. Of particular interest are sun protection creams, sun protection lotions, sun protection milk and sun protection preparations in the form of a spray.

Of special importance as cosmetic preparations for the hair are the above-mentioned preparations for hair treatment, especially hair-washing preparations in the form of shampoos, hair conditioners, hair-care preparations, e.g. pretreatment preparations, hair tonics, styling creams, styling gels, pomades, hair rinses, treatment packs, intensive hair treatments, hair-straightening preparations, liquid hair-setting preparations, hair foams and hairsprays. Of special interest are hair-washing preparations in the form of shampoos.

A shampoo has, for example, the following composition: from 0.01 to 5% by weight of a UV absorber composition according to the invention, 12.0% by weight of sodium laureth-2-sulfate, 4.0% by weight of cocamidopropyl betaine, 3.0% by weight of sodium chloride, and water ad 100%.

Other typical ingredients in such formulations are preservatives, bactericides and bacteriostatic agents, perfumes, dyes, pigments, thickening agents, moisturizing agents, humectants, fats, oils, waxes or other typical ingredients of cosmetic and personal care formulations such as alcohols, poly-alcohols, polymers, electrolytes, organic solvents, silicon derivatives, emollients, emulsifiers or emulsifying surfactants, surfactants, dispersing agents, antioxidants, anti-irritants and anti-inflammatory agents etc.

The cosmetic preparation according to the invention is distinguished by excellent protection of human skin against the damaging effect of sunlight.

### Method to Assess in Vitro Sun Protection Factor Measurement (SPF)

End-product application rate 1.4 mg/cm² on PMMA plates (Helioplates®) UV Transmittance analysis with Labsphere UV-1000S Transmittance Analyser

$$SPF = \frac{{\int }_{290 n m}^{400 n m} {E}_{λ} \cdot {S}_{λ} \cdot d λ}{{\int }_{290 n m}^{400 n m} {E}_{λ} \cdot {S}_{λ} \cdot {T}_{λ} \cdot d λ}$$

wherein E_{λ}=erythema action spectrum; S_{λ}=solar spectral irradiance and T_{λ}=spectral transmittance of the sample.

### Method to Assess in Vitro UVA Protection Factor (UVA PF)

End-product application rate 1.2 mg/cm² on PMMA plates (Helioplates®)

UV Transmittance analysis with Labsphere UV-1000S Transmittance Analyser

Pre-irradiation step (to take the sun care product photostability into account) via a solar simulator suc as Atlas Suntest CPS+

$$PFUVA = \frac{{{\sum }_{320}}^{400} Δλ}{{{\sum }_{320}}^{400} {T}_{λ} \cdot {Δ}_{λ}} = \frac{1}{{T}_{m}}$$

Wherein T_{λ}=sunscreen product transmittance at wave length λ and Tₘ=mean arithmetical value of Transmittance data in the UVA range.

### B. Cosmetic Formulations

### EXAMPLE B1: Emulsion High Protection

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| Part | Cyclomethicone | 7.00 | 7.00 | 7.00 | 7.00 | 7.00 | 7.00 | 7.00 | 7.00 | 7.00 |
| A | Ethylhexyl Palmitate | 5.00 | 5.00 | 5.00 | 5.00 | 5.00 | 5.00 | 5.00 | 5.00 | 5.00 |
| | Glyceryl Stearate | 1.50 | 1.50 | 1.50 | 1.50 | 1.50 | 1.50 | 1.50 | 1.50 | 1.50 |
| | Octocrylene | 1.00 | 1.00 | 1.00 | 5.00 | 6.00 | 8.00 | 4.00 | 3.00 | 4.00 |
| | Ethylhexyl Triazone | 6.00 | 6.00 | 6.00 | 2.00 | 3.00 | 4.00 | 2.00 | 3.00 | 4.00 |
| | Potassium Cetyl Phosphate | 1.80 | 1.80 | 1.80 | 1.80 | 1.80 | 1.80 | 1.80 | 1.80 | 1.80 |
| | VP/Eicosene Copolymer | 0.60 | 0.60 | 0.60 | 0.60 | 0.60 | 0.60 | 0.60 | 0.60 | 0.60 |
| Part | Organic micro-particulate UV | 2.00 | 4.00 | 6.00 | 8.00 | 10.00 | 14.00 | 16.00 | 18.00 | 20.00 |
| B | absorber of formula (3), (5) or (7) | | | | | | | | | |
| Part | Aqua | Qs to 100 | Qs to 100 | Qs to 100 | Qs to 100 | Qs to 100 | Qs to 100 | Qs to 100 | Qs to 100 | Qs to 100 |
| C | Acrylates/Palmeth-25 Acrylate | 0.25 | 0.25 | 0.25 | 0.25 | 0.25 | 0.25 | 0.25 | 0.25 | 0.25 |
| | Copolymer | | | | | | | | | |
| | Phenyl benzimidazole sulfonic | 1.00 | 2.00 | 3.00 | 1.00 | 2.00 | 3.00 | 2.00 | 2.00 | 2.00 |
| | acid | | | | | | | | | |
| | Glycerin | 6.00 | 6.00 | 6.00 | 6.00 | 6.00 | 6.00 | 6.00 | 6.00 | 6.00 |
| | Disodium EDTA | 0.20 | 0.20 | 0.20 | 0.20 | 0.20 | 0.20 | 0.20 | 0.20 | 0.20 |
| Part | Phenoxyethanol (and) Methyl- | 0.70 | 0.70 | 0.70 | 0.70 | 0.70 | 0.70 | 0.70 | 0.70 | 0.70 |
| D | paraben (and) Ethylparaben (and) | | | | | | | | | |
| | Butylparaben (and) Propylparaben | | | | | | | | | |
| | (and) Isobutylparaben | | | | | | | | | |
| | Tocopheryl Acetate | 0.30 | 0.30 | 0.30 | 0.30 | 0.30 | 0.30 | 0.30 | 0.30 | 0.30 |

| | | | | | | | | | |
|---|---|---|---|---|---|---|---|---|---|
| In vitro SPF measured according the | 18.5 | 25.1 | 30.9 | 22.4 | 29.2 | 41.7 | 25.2 | 29 | 35.2 |
| method described | | | | | | | | | |
| UVA PF measured according the | 2.5 | 3.3 | 4 | 5.2 | 5.9 | 7.4 | 7.4 | 7.8 | 8.4 |
| method described | | | | | | | | | |

### Manufacturing Instruction:

Part A is prepared by incorporating all ingredients, then stirred under moderate speed and heated to 75° C. Part C is prepared and heated to 75° C. At this temperature, part A is poured into part C under progressive stirring speed. Below 65° C. the ingredients of part B are added separately. After cooling down under moderate stirring to 40° C. part D is added. The mixture is homogenized for 30 sec at 10000 rpm.

### EXAMPLE B2: Sun Milk

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| Part | C12-15 Alkyl Benzoate | 4.00 | 4.00 | 4.00 | 4.00 | 4.00 | 4.00 | 4.00 | 4.00 | 4.00 |
| A | Octocrylene | 7.50 | 7.50 | 7.50 | 7.50 | 7.50 | 7.50 | 7.50 | 7.50 | 7.50 |
| | Isohexadecane | 3.00 | 3.00 | 3.00 | 3.00 | 3.00 | 3.00 | 3.00 | 3.00 | 3.00 |
| | Cyclopentasiloxane | 4.00 | 4.00 | 4.00 | 4.00 | 4.00 | 4.00 | 4.00 | 4.00 | 4.00 |
| | Stearic Acid | 0.50 | 0.50 | 0.50 | 0.50 | 0.50 | 0.50 | 0.50 | 0.50 | 0.50 |
| | PEG-100 Stearate (and) Glyceryl | 0.80 | 0.80 | 0.80 | 0.80 | 0.80 | 0.80 | 0.80 | 0.80 | 0.80 |
| | Stearate | | | | | | | | | |
| | Potassium Cetyl Phosphate | 1.30 | 1.30 | 1.30 | 1.30 | 1.30 | 1.30 | 1.30 | 1.30 | 1.30 |
| | Butyl Methoxydibenzoylmethane | 6.00 | 4.00 | 2.00 | 3.00 | 5.00 | 2.00 | 3.00 | 2.00 | 1.00 |
| | Ethylhexyl Methoxycinnamate | 6.00 | 5.00 | 4.00 | 6.00 | 5.00 | 4.00 | 3.00 | 4.00 | 3.00 |
| | Titanium Dioxide | 8.00 | 3.00 | 4.00 | 6.00 | 3.00 | 4.00 | 2.00 | 3.00 | 2.00 |
| | PVP/Eicosene Copolymer | 1.00 | 1.00 | 1.00 | 1.00 | 1.00 | 1.00 | 1.00 | 1.00 | 1.00 |
| Part | Organic micro-particulate UV | 2.00 | 4.00 | 6.00 | 8.00 | 10.0 | 14.0 | 16.0 | 18.0 | 20.0 |
| B | absorber of formula (3), (5) or (7) | | | | | | | | | |
| Part | Aqua | Qs to 100 | Qs to 100 | Qs to 100 | Qs to 100 | Qs to 100 | Qs to 100 | Qs to 100 | Qs to 100 | Qs to 100 |
| C | Glycerin | 4.00 | 4.00 | 4.00 | 4.00 | 4.00 | 4.00 | 4.00 | 4.00 | 4.00 |
| | Propylene Glycol | 5.00 | 5.00 | 5.00 | 5.00 | 5.00 | 5.00 | 5.00 | 5.00 | 5.00 |
| | Xanthan Gum | 0.20 | 0.20 | 0.20 | 0.20 | 0.20 | 0.20 | 0.20 | 0.20 | 0.20 |
| | Acrylates/C10-30 Alkyl Acrylate | 0.15 | 0.15 | 0.15 | 0.15 | 0.15 | 0.15 | 0.15 | 0.15 | 0.15 |
| | Crosspolymer | | | | | | | | | |
| | Disodium EDTA | 0.15 | 0.15 | 0.15 | 0.15 | 0.15 | 0.15 | 0.15 | 0.15 | 0.15 |
| Part | Triethanolamine | Qs | Qs | Qs | Qs | Qs | Qs | Qs | Qs | Qs |
| D | Dimethicone | 0.40 | 0.40 | 0.40 | 0.40 | 0.40 | 0.40 | 0.40 | 0.40 | 0.40 |
| | Phenoxyethanol (and) Methyl- | 0.70 | 0.70 | 0.70 | 0.70 | 0.70 | 0.70 | 0.70 | 0.70 | 0.70 |
| | paraben (and) Ethylparaben (and) | | | | | | | | | |
| | Butylparaben (and) Propylparaben | | | | | | | | | |
| | (and) Isobutylparaben | | | | | | | | | |
| | Tocopheryl Acetate | 0.90 | 0.90 | 0.90 | 0.90 | 0.90 | 0.90 | 0.90 | 0.90 | 0.90 |

| | | | | | | | | | |
|---|---|---|---|---|---|---|---|---|---|
| In vitro SPF measured according the | 40.9 | 32.2 | 38.2 | 49.7 | 45.5 | 58.1 | 57.7 | 65.6 | 66.6 |
| method described | | | | | | | | | |
| UVA PF measured according the | 9.8 | 9.3 | 10.1 | 11.6 | 11.5 | 12.9 | 14.1 | 13.9 | 13.2 |
| method described | | | | | | | | | |

### Manufacturing Instruction:

Part A is prepared by incorporating all ingredients, then stirred under moderate speed and heated to 75° C. Part C is prepared and heated to 75° C. At this temperature, part A is poured into part C under progressive stirring speed. Below 65° C. the ingredients of part B are added separately. After cooling down under moderate stirring to 40° C. part D is added. Then the pH is checked and adjusted with triethanolamine.

### EXAMPLE B3: Sun Milk

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| Part | C12-15 Alkyl Benzoate | 4.00 | 4.00 | 4.00 | 4.00 | 4.00 | 4.00 | 4.00 | 4.00 | 4.00 |
| A | Butylen Glycol | 3.00 | 3.00 | 3.00 | 3.00 | 3.00 | 3.00 | 3.00 | 3.00 | 3.00 |
| | Dicaprylate/Dicaprate | | | | | | | | | |
| | Caprylic/Capric Triglyceride | 3.00 | 3.00 | 3.00 | 3.00 | 3.00 | 3.00 | 3.00 | 3.00 | 3.00 |
| | Octyldodecanol | 4.00 | 4.00 | 4.00 | 4.00 | 4.00 | 4.00 | 4.00 | 4.00 | 4.00 |
| | Dicaprylyl Ether | 3.50 | 3.50 | 3.50 | 3.50 | 3.50 | 3.50 | 3.50 | 3.50 | 3.50 |
| | Dicaprylyl Carbonate | 2.10 | 2.10 | 2.10 | 2.10 | 2.10 | 2.10 | 2.10 | 2.10 | 2.10 |
| | Glyceryl Stearate Citrate | 2.00 | 2.00 | 2.00 | 2.00 | 2.00 | 2.00 | 2.00 | 2.00 | 2.00 |
| | Diethylhexyl Butamido Triazine | 4.00 | 3.00 | 5.00 | 1.50 | 3.00 | 2.00 | 1.00 | 1.00 | 2.00 |
| | Bis-Ethylhexyloxyphenol | 1.00 | 2.00 | 3.00 | 2.50 | 3.00 | 2.00 | 1.00 | 3.00 | 1.00 |
| | Methoxyphenyl Triazine | | | | | | | | | |
| | Ethylhexyl Methoxycinnamate | 4.00 | 6.00 | 10.00 | 8.00 | 7.00 | 6.00 | 4.00 | 5.00 | 5.00 |
| | Titanium Dioxide | 7.00 | 8.00 | 2.00 | — | 1.50 | 1.00 | 3.00 | 1.00 | 2.00 |
| | Stearyl Alcohol | 1.50 | 1.50 | 1.50 | 1.50 | 1.50 | 1.50 | 1.50 | 1.50 | 1.50 |
| | Hydrogenated Coco-Glycerides | 1.40 | 1.40 | 1.40 | 1.40 | 1.40 | 1.40 | 1.40 | 1.40 | 1.40 |
| | PVP/Hexadecene Copolymer | 0.50 | 0.50 | 0.50 | 0.50 | 0.50 | 0.50 | 0.50 | 0.50 | 0.50 |
| Part | Organic micro-particulate UV | 2.00 | 4.00 | 6.00 | 8.00 | 10.00 | 14.00 | 16.00 | 18.00 | 20.00 |
| B | absorber of formula (3), (5) or (7) | | | | | | | | | |
| Part | Aqua | Qs to 100 | Qs to 100 | Qs to 100 | Qs to 100 | Qs to 100 | Qs to 100 | Qs to 100 | Qs to 100 | Qs to 100 |
| C | Glycerin | 6.00 | 6.00 | 6.00 | 6.00 | 6.00 | 6.00 | 6.00 | 6.00 | 6.00 |
| | Xanthan Gum | 0.25 | 0.25 | 0.25 | 0.25 | 0.25 | 0.25 | 0.25 | 0.25 | 0.25 |
| | Acrylates/C10-30 Alkyl Acrylate | 0.15 | 0.15 | 0.15 | 0.15 | 0.15 | 0.15 | 0.15 | 0.15 | 0.15 |
| | Crosspolymer | | | | | | | | | |
| | Trisodium EDTA | 0.20 | 0.20 | 0.20 | 0.20 | 0.20 | 0.20 | 0.20 | 0.20 | 0.20 |
| Part | Alcohol Denatured | 4.00 | 4.00 | 4.00 | 4.00 | 4.00 | 4.00 | 4.00 | 4.00 | 4.00 |
| D | | | | | | | | | | |
| Part | Sodium Hydroxide | Qs | Qs | Qs | Qs | Qs | Qs | Qs | Qs | Qs |
| E | Phenoxyethanol (and) Methyl- | 0.70 | 0.70 | 0.70 | 0.70 | 0.70 | 0.70 | 0.70 | 0.70 | 0.70 |
| | paraben (and) Ethylparaben (and) | | | | | | | | | |
| | Butylparaben (and) Propylparaben | | | | | | | | | |
| | (and) Isobutylparaben | | | | | | | | | |
| | Tocopheryl Acetate | 1.00 | 1.00 | 1.00 | 1.00 | 1.00 | 1.00 | 1.00 | 1.00 | 1.00 |

| | | | | | | | | | |
|---|---|---|---|---|---|---|---|---|---|
| In vitro SPF measured according the | 37.4 | 49.6 | 52.1 | 28 | 41.7 | 30.9 | 26 | 29.3 | 30.3 |
| method described | | | | | | | | | |
| UVA PF measured according the | 7.7 | 11.2 | 10.1 | 8.5 | 11.1 | 10.4 | 10.6 | 13 | 11 |
| method described | | | | | | | | | |

### Manufacturing Instruction:

Part A is prepared by incorporating all ingredients, then stirred under moderate speed and heated to 75° C. Part C is prepared and heated to 75° C. At this temperature, part A is poured into part C under progressive stirring speed. Below 65° C. the ingredients of part B are added separately. After cooling down under moderate stirring to 40° C. part D is added. Then, part E is added to the emulsion formed and the pH is checked and adjusted with triethanolamine.

### EXAMPLE B4: Water Resistant Sunscreen Emulsion

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| Part | Polyglyceryl-10 Pentastearate | 2.50 | 2.50 | 2.50 | 2.50 | 2.50 | 2.50 | 2.50 | 2.50 | 2.50 |
| A | (and) Behenyl Alcohol (and) | | | | | | | | | |
| | Sodium Stearoyl Lactylate | | | | | | | | | |
| | VP/Eicosene Copolymer | 1.50 | 1.50 | 1.50 | 1.50 | 1.50 | 1.50 | 1.50 | 1.50 | 1.50 |
| | Stearyl Alcohol | 1.50 | 1.50 | 1.50 | 1.50 | 1.50 | 1.50 | 1.50 | 1.50 | 1.50 |
| | Squalane | 4.00 | 4.00 | 4.00 | 4.00 | 4.00 | 4.00 | 4.00 | 4.00 | 4.00 |
| | C12-15 Alkyl Benzoate | 5.50 | 5.50 | 5.50 | 5.50 | 5.50 | 5.50 | 5.50 | 5.50 | 5.50 |
| | Bis-Ethylhexyloxyphenol | 4.00 | 3.00 | 2.00 | 6.00 | 2.50 | 3.00 | 1.50 | 2.00 | 1.00 |
| | Methoxyphenyl Triazine | | | | | | | | | |
| | Ethylhexyl Methoxycinnamate | 4.00 | 6.00 | 5.00 | 8.00 | 4.00 | 3.00 | 2.00 | 1.50 | 1.00 |
| | Titanium Dioxide | 1.00 | 4.00 | 6.00 | 8.00 | 2.00 | 4.00 | 6.00 | 3.00 | 1.50 |
| | Octocrylene | 10.0 | 4.00 | 5.00 | 4.00 | 4.00 | 5.00 | 3.00 | 4.00 | 2.00 |
| Part | Organic micro-particulate UV | 2.00 | 4.00 | 6.00 | 8.00 | 10.0 | 14.0 | 16.0 | 18.0 | 20.0 |
| B | absorber of formula (3), (5) or (7) | | | | | | | | | |
| Part | Water | qs to 100 | qs to 100 | qs to 100 | qs to 100 | qs to 100 | qs to 100 | qs to 100 | qs to 100 | qs to 100 |
| C | Glycerin | 1.80 | 1.80 | 1.80 | 1.80 | 1.80 | 1.80 | 1.80 | 1.80 | 1.80 |
| | Steareth-10 Allyl Ether/Acrylates | 0.80 | 0.80 | 0.80 | 0.80 | 0.80 | 0.80 | 0.80 | 0.80 | 0.80 |
| | Copolymer | | | | | | | | | |
| | VP/Hexadecene Copolymer | 2.70 | 2.70 | 2.70 | 2.70 | 2.70 | 2.70 | 2.70 | 2.70 | 2.70 |
| Part | Cyclomethicone | 1.50 | 1.50 | 1.50 | 1.50 | 1.50 | 1.50 | 1.50 | 1.50 | 1.50 |
| D | Phenoxyethanol (and) Methyl- | 0.70 | 0.70 | 0.70 | 0.70 | 0.70 | 0.70 | 0.70 | 0.70 | 0.70 |
| | paraben (and) Ethylparaben (and) | | | | | | | | | |
| | Butylparaben (and) Propylparaben | | | | | | | | | |
| | (and) Isobutylparaben | | | | | | | | | |
| | Aqua (and) Tocopheryl Acetate | 3.50 | 3.50 | 3.50 | 3.50 | 3.50 | 3.50 | 3.50 | 3.50 | 3.50 |
| | (and) Caprylic/Capric Triglyceride | | | | | | | | | |
| | (and) Polysorbate 80 (and) | | | | | | | | | |
| | Lecithin | | | | | | | | | |
| | Fragrance | qs | qs | qs | qs | qs | qs | qs | qs | qs |
| | Water (and) Sodium Hydroxide | qs | qs | qs | qs | qs | qs | qs | qs | qs |

| | | | | | | | | | |
|---|---|---|---|---|---|---|---|---|---|
| In vitro SPF measured according the | 36 | 36.6 | 39.5 | 69.1 | 28 | 37.7 | 33.1 | 26.8 | 14 |
| method described | | | | | | | | | |
| UVA PF measured according the | 9.7 | 11 | 11.6 | 20.6 | 11.1 | 14.7 | 13.7 | 13.2 | 10.8 |
| method described | | | | | | | | | |

### Manufacturing Instruction:

Part A is prepared by incorporating all ingredients, then stirred under moderate speed and heated to 75° C. Part C is prepared and heated to 75° C. At this temperature, part A is poured into part C under progressive stirring speed. Below 65° C. the ingredients of part B are added separately. After cooling down under moderate stirring to 40° C. part D is added. Then the pH is checked and adjusted with triethanolamine.

### EXAMPLE B5: O/W Daily Care

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| Part | Cetearyl Alcohol (and) Dicetyl | 1.50 | 1.50 | 1.50 | 1.50 | 1.50 | 1.50 | 1.50 | 1.50 | 1.50 |
| A | Phosphate (and) Ceteth-10 | | | | | | | | | |
| | Phosphate | | | | | | | | | |
| | C12-15 Alkyl Benzoate | 2.00 | 2.00 | 2.00 | 2.00 | 2.00 | 2.00 | 2.00 | 2.00 | 2.00 |
| | Dicaprylyl Ether | 3.00 | 3.00 | 3.00 | 3.00 | 3.00 | 3.00 | 3.00 | 3.00 | 3.00 |
| | Ethoxydiglycol Oleate | 2.00 | 2.00 | 2.00 | 2.00 | 2.00 | 2.00 | 2.00 | 2.00 | 2.00 |
| | Stearic Acid | 1.00 | 1.00 | 1.00 | 1.00 | 1.00 | 1.00 | 1.00 | 1.00 | 1.00 |
| | Sodium Acrylates Copolymer | 2.00 | 2.00 | 2.00 | 2.00 | 2.00 | 2.00 | 2.00 | 2.00 | 2.00 |
| | (and) Glycine Soja (and) PPG-1 | | | | | | | | | |
| | Trideceth-6 | | | | | | | | | |
| | Squalane | 3.50 | 3.50 | 3.50 | 3.50 | 3.50 | 3.50 | 3.50 | 3.50 | 3.50 |
| | Drometrizole trisiloxane | 10.00 | 8.00 | 7.00 | 5.00 | 4.00 | 3.00 | 2.00 | 1.50 | 1.00 |
| | Diethylamino hydroxybenzoyl | 5.00 | 3.00 | 2.00 | 2.50 | 3.00 | 1.00 | 2.00 | 3.00 | 1.50 |
| | Hexylbenzoate | | | | | | | | | |
| | Ethylhexyl Triazone | 3.00 | 1.50 | 2.00 | 2.50 | 1.00 | 1.00 | 1.00 | 1.50 | 2.00 |
| Part | Organic micro-particulate UV | 2.00 | 4.00 | 6.00 | 8.00 | 10.0 | 14.0 | 16.0 | 18.0 | 20.0 |
| B | absorber of formula (3), (5) or (7) | | | | | | | | | |
| Part | Aqua | qs to 100 | qs to 100 | qs to 100 | qs to 100 | qs to 100 | qs to 100 | qs to 100 | qs to 100 | qs to 100 |
| C | | | | | | | | | | |
| Part | Diazolidinyl Urea (and) | 0.15 | 0.15 | 0.15 | 0.15 | 0.15 | 0.15 | 0.15 | 0.15 | 0.15 |
| D | Iodopropynyl Butylcarbamate | | | | | | | | | |
| | Propylene Glycol | 2.50 | 2.50 | 2.50 | 2.50 | 2.50 | 2.50 | 2.50 | 2.50 | 2.50 |
| | Aqua | 10.0 | 10.0 | 10.0 | 10.0 | 10.0 | 10.0 | 10.0 | 10.0 | 10.0 |
| Part | Cyclopentasiloxane, Dimethiconol | 2.00 | 2.00 | 2.00 | 2.00 | 2.00 | 2.00 | 2.00 | 2.00 | 2.00 |
| E | Ethoxydiglycol | 5.00 | 5.00 | 5.00 | 5.00 | 5.00 | 5.00 | 5.00 | 5.00 | 5.00 |
| | Cyclopentasiloxane (and) | 2.00 | 2.00 | 2.00 | 2.00 | 2.00 | 2.00 | 2.00 | 2.00 | 2.00 |
| | Dimethicone/Vinyl-dimethicone | | | | | | | | | |
| | Crosspolymer | | | | | | | | | |
| | Sodium Hydroxide | 0.10 | 0.10 | 0.10 | 0.10 | 0.10 | 0.10 | 0.10 | 0.10 | 0.10 |
| | Citric Acid (and) Silver Citrate | 0.10 | 0.10 | 0.10 | 0.10 | 0.10 | 0.10 | 0.10 | 0.10 | 0.10 |
| | Sclerotium Gum | 0.10 | 0.10 | 0.10 | 0.10 | 0.10 | 0.10 | 0.10 | 0.10 | 0.10 |

| | | | | | | | | | |
|---|---|---|---|---|---|---|---|---|---|
| In vitro SPF measured according the | 29.6 | 16.5 | 17.4 | 18.4 | 11 | 9.6 | 9.5 | 12 | 13.1 |
| method described | | | | | | | | | |
| UVA PF measured according the | 15.8 | 11.8 | 10.4 | 11 | 11.9 | 9.3 | 10.9 | 12.8 | 10.6 |
| method described | | | | | | | | | |

### Manufacturing Instruction:

Part A is prepared by incorporating all ingredients, then stirred under moderate speed and heated to 75° C. Part C is prepared and heated to 75° C. At this temperature, part A is poured into part C under progressive stirring speed. Below 65° C. the ingredients of part B are added separately. After cooling down under moderate stirring to 40° C. part D is added. Then, part E is added to the emulsion formed and the pH is checked and adjusted with sodium hydroxide.

### EXAMPLE B6: Every Day Lotion

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| Part | Stearyl Phosphate | 5.00 | 5.00 | 5.00 | 5.00 | 5.00 | 5.00 | 5.00 | 5.00 | 5.00 |
| A | Tricontanyl PVP | 1.00 | 1.00 | 1.00 | 1.00 | 1.00 | 1.00 | 1.00 | 1.00 | 1.00 |
| | Ethoxydiglycol Oleate | 3.00 | 3.00 | 3.00 | 3.00 | 3.00 | 3.00 | 3.00 | 3.00 | 3.00 |
| | Squalane | 5.00 | 5.00 | 5.00 | 5.00 | 5.00 | 5.00 | 5.00 | 5.00 | 5.00 |
| | C12-15 Alkyl Benzoate | 5.00 | 5.00 | 5.00 | 5.00 | 5.00 | 5.00 | 5.00 | 5.00 | 5.00 |
| | Glyceryl Stearate | 2.00 | 2.00 | 2.00 | 2.00 | 2.00 | 2.00 | 2.00 | 2.00 | 2.00 |
| | Cetyl Alcohol | 2.00 | 2.00 | 2.00 | 2.00 | 2.00 | 2.00 | 2.00 | 2.00 | 2.00 |
| | Diethylhexyl Butamido | 4.00 | 5.00 | 3.00 | 1.00 | 1.00 | 3.00 | 2.00 | 1.50 | 1.00 |
| | Triazone | | | | | | | | | |
| | Ethylhexyl | 6.00 | 8.00 | 4.00 | 3.00 | 2.00 | 4.00 | 5.00 | 3.00 | 3.00 |
| | Methoxycinnamate | | | | | | | | | |
| | Titanium Dioxide | 4.00 | 5.00 | 3.00 | 2.50 | 2.00 | 2.00 | 1.00 | 1.50 | 1.00 |
| Part | Organic micro-particulate UV | 2.00 | 4.00 | 6.00 | 8.00 | 10.00 | 14.00 | 16.00 | 18.00 | 20.00 |
| B | absorber of formula (3), (5) or (7) | | | | | | | | | |
| Part | Aqua | qs to 100 | qs to 100 | qs to 100 | qs to 100 | qs to 100 | qs to 100 | qs to 100 | qs to 100 | qs to 100 |
| C | Steareth-10 Allyl Ether/Acrylates | 0.50 | 0.50 | 0.50 | 0.50 | 0.50 | 0.50 | 0.50 | 0.50 | 0.50 |
| | Copolymer | | | | | | | | | |
| | Glycerin | 2.50 | 2.50 | 2.50 | 2.50 | 2.50 | 2.50 | 2.50 | 2.50 | 2.50 |
| | Diazolidinyl Urea (and) | 0.15 | 0.15 | 0.15 | 0.15 | 0.15 | 0.15 | 0.15 | 0.15 | 0.15 |
| | Iodopropynyl Butylcarbamate | | | | | | | | | |
| | Disodium Phenyl Dibenz- | 7.00 | 5.00 | 4.00 | 3.00 | 2.00 | 3.00 | 1.50 | 1.00 | 1.00 |
| | imidazole tetrasulfonate | | | | | | | | | |
| | Sodium Lauroyl Glutamate | 0.70 | 0.70 | 0.70 | 0.70 | 0.70 | 0.70 | 0.70 | 0.70 | 0.70 |
| Part | Cyclopentasiloxane (and) | 1.50 | 1.50 | 1.50 | 1.50 | 1.50 | 1.50 | 1.50 | 1.50 | 1.50 |
| D | Dimethiconol | | | | | | | | | |
| | Triethanolamine | 1.85 | 1.85 | 1.85 | 1.85 | 1.85 | 1.85 | 1.85 | 1.85 | 1.85 |
| | Citric Acid (and) Silver Citrate | 0.10 | 0.10 | 0.10 | 0.10 | 0.10 | 0.10 | 0.10 | 0.10 | 0.10 |
| | Sclerotium Gum | 0.10 | 0.10 | 0.10 | 0.10 | 0.10 | 0.10 | 0.10 | 0.10 | 0.10 |

| | | | | | | | | | |
|---|---|---|---|---|---|---|---|---|---|
| In vitro SPF measured according the | 50.9 | 56.6 | 38.7 | 25.7 | 19.8 | 36.8 | 27.1 | 24.1 | 20.5 |
| method described | | | | | | | | | |
| UVA PF measured according the | 9.9 | 10.8 | 10.7 | 10.7 | 10.2 | 13 | 10.9 | 11.5 | 11.7 |
| method described | | | | | | | | | |

### Manufacturing Instruction:

Part A is prepared by incorporating all ingredients, then stirred under moderate speed and heated to 75° C. Part C is prepared and heated to 75° C. At this temperature, part A is poured into part C under progressive stirring speed. Below 65° C. the ingredients of part B are added separately. After cooling down under moderate stirring to 40° C. part D is added. Then the pH is checked and adjusted with triethanolamine.

### EXAMPLE B7: Sun Cream

Example B7: Sun Cream

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| Part A | Tribehenin PEG-20 esters | 5.00 | 5.00 | 5.00 | 5.00 | 5.00 | 5.00 | 5.00 | 5.00 | 5.00 |
| | Dibutyl adipate | 5.00 | 5.00 | 5.00 | 5.00 | 5.00 | 5.00 | 5.00 | 5.00 | 5.00 |
| | PPG-2 Myristyl Ether | 5.00 | 5.00 | 5.00 | 5.00 | 5.00 | 5.00 | 5.00 | 5.00 | 5.00 |
| | Propionate | | | | | | | | | |
| | Octocrylene | 10.00 | 4.00 | 5.00 | 4.00 | 4.00 | 5.00 | 3.00 | 4.00 | 2.00 |
| | Ethylhexyl Triazone | 3.00 | 1.50 | 2.00 | 2.50 | 1.00 | 1.00 | 1.00 | 1.50 | 2.00 |
| | Bis-Ethylhexyloxyphenol | 4.00 | 3.00 | 2.00 | 6.00 | 2.50 | 3.00 | 1.50 | 2.00 | 1.00 |
| | Methoxyphenyl Triazine | | | | | | | | | |
| | Ethylhexyl Methoxycinnamate | 6.00 | 8.00 | 4.00 | 3.00 | 2.00 | 4.00 | 5.00 | 3.00 | 3.00 |
| | Butyl Methoxy Dibenzoyl | 3.00 | 2.00 | 2.50 | 3.00 | 2.00 | 1.50 | 1.50 | 1.00 | 1.00 |
| | Methane | | | | | | | | | |
| Part B | Organic micro-particulate UV | 2.00 | 4.00 | 6.00 | 8.00 | 10.0 | 14.0 | 16.0 | 18.0 | 20.0 |
| | absorber of formula (3), (5) or | | | | | | | | | |
| | (7) | | | | | | | | | |
| Part C | Water | Qs to | Qs to | Qs to | Qs to | Qs to | Qs to | Qs to | Qs to | Qs to |
| | | 100 | 100 | 100 | 100 | 100 | 100 | 100 | 100 | 100 |
| | PVP/dimethylconylacrylate/polycarbamyl/ | 3.00 | 3.00 | 3.00 | 3.00 | 3.00 | 3.00 | 3.00 | 3.00 | 3.00 |
| | polyglycol ester | | | | | | | | | |
| | Disodium EDTA | 0.20 | 0.20 | 0.20 | 0.20 | 0.20 | 0.20 | 0.20 | 0.20 | 0.20 |
| | Sclerotium Gum | 2.00 | 2.00 | 2.00 | 2.00 | 2.00 | 2.00 | 2.00 | 2.00 | 2.00 |
| | Ammonium | 0.50 | 0.50 | 0.50 | 0.50 | 0.50 | 0.50 | 0.50 | 0.50 | 0.50 |
| | Acryldimethyltaurate/Beneth-25 | | | | | | | | | |
| | Methacrylate Crosspolymer. | | | | | | | | | |
| Part D | Cyclopentasiloxane (and) | 4.00 | 4.00 | 4.00 | 4.00 | 4.00 | 4.00 | 4.00 | 4.00 | 4.00 |
| | cyclohexasiloxane | | | | | | | | | |
| | Aqua (and) Tocopheryl Acetate | 0.50 | 0.50 | 0.50 | 0.50 | 0.50 | 0.50 | 0.50 | 0.50 | 0.50 |
| | (and) Caprylic/Capric | | | | | | | | | |
| | Triglyceride (and) Polysorbate | | | | | | | | | |
| | 80 (and) Lecithin | | | | | | | | | |
| | Phenoxyethanol (and) Methyl- | 1.00 | 1.00 | 1.00 | 1.00 | 1.00 | 1.00 | 1.00 | 1.00 | 1.00 |
| | paraben (and) Ethylparaben | | | | | | | | | |
| | (and) Butylparaben (and) Propylparaben | | | | | | | | | |
| | (and) | | | | | | | | | |
| | Isobutylparaben | | | | | | | | | |
| | Sodium Hydroxide (and) Water | qs | qs | qs | qs | qs | qs | qs | qs | qs |

| | | | | | | | | | |
|---|---|---|---|---|---|---|---|---|---|
| In vitro SPF measured according the | 54.3 | 34.4 | 30.9 | 53.2 | 24.1 | 29.3 | 25.2 | 29.2 | 24.3 |
| method described | | | | | | | | | |
| UVA PF measured according the | 12.6 | 9.8 | 10.1 | 17.7 | 12.6 | 14.6 | 10.9 | 12.8 | 10.9 |
| method described | | | | | | | | | |

### Manufacturing Instruction:

Part A is prepared by incorporating all ingredients, then stirred under moderate speed and heated to 75° C. Part C is prepared and heated to 75° C. At this temperature, part A is poured into part C under progressive stirring speed. Below 65° C. the ingredients of part B are added separately. After cooling down under moderate stirring to 40° C. part D is added. Then the pH is checked and adjusted with sodium hydroxide

### EXAMPLE B8: Daily Care Lotion

Example B8: Daily Care Lotion

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| Part A | Polyglyceryl Methyl Glucose | 2.50 | 2.50 | 2.50 | 2.50 | 2.50 | 2.50 | 2.50 | 2.50 | 2.50 |
| | Distearate | | | | | | | | | |
| | Cetearyl Alcohol | 2.00 | 2.00 | 2.00 | 2.00 | 2.00 | 2.00 | 2.00 | 2.00 | 2.00 |
| | Octyl Stearate | 3.00 | 3.00 | 3.00 | 3.00 | 3.00 | 3.00 | 3.00 | 3.00 | 3.00 |
| | Caprylic/Capric Triglyceride | 4.00 | 4.00 | 4.00 | 4.00 | 4.00 | 4.00 | 4.00 | 4.00 | 4.00 |
| | Isohexadecane | 4.00 | 4.00 | 4.00 | 4.00 | 4.00 | 4.00 | 4.00 | 4.00 | 4.00 |
| | Polysilicone-15 | 8.00 | 6.00 | 4.00 | 3.50 | 2.50 | 2.00 | 1.50 | 1.00 | 1.00 |
| | Bis-Ethylhexyloxyphenol | 4.00 | 3.00 | 2.00 | 6.00 | 2.50 | 3.00 | 1.50 | 2.00 | 1.00 |
| | Methoxyphenyl Triazine | | | | | | | | | |
| | Ethylhexyl Triazone | 3.00 | 1.50 | 2.00 | 2.50 | 1.00 | 1.00 | 1.00 | 1.50 | 2.00 |
| Part B | Organic micro-particulate UV | 2.00 | 4.00 | 6.00 | 8.00 | 10.0 | 14.0 | 16.0 | 18.0 | 20.0 |
| | absorber of formula (3), (5) or | | | | | | | | | |
| | (7) | | | | | | | | | |
| Part C | Aqua | qs to | qs to | qs to | qs to | qs to | qs to | qs to | qs to | qs to |
| | | 100 | 100 | 100 | 100 | 100 | 100 | 100 | 100 | 100 |
| | Terephthalidene | 8.00 | 6.00 | 4.00 | 3.00 | 2.00 | 2.00 | 1.00 | 1.50 | 1.00 |
| | Dicamphorsulfonic acid | | | | | | | | | |
| | Glycerin | 5.00 | 5.00 | 5.00 | 5.00 | 5.00 | 5.00 | 5.00 | 5.00 | 5.00 |
| Part D | Phenoxyethanol (and) Methyl- | 0.50 | 0.50 | 0.50 | 0.50 | 0.50 | 0.50 | 0.50 | 0.50 | 0.50 |
| | paraben (and) Butylparaben | | | | | | | | | |
| | (and) Ethylparaben (and) Propylparaben | | | | | | | | | |
| | Cyclomethicone (and) | 3.00 | 3.00 | 3.00 | 3.00 | 3.00 | 3.00 | 3.00 | 3.00 | 3.00 |
| | Dimethicone | | | | | | | | | |
| | Steareth-10 Allyl | 0.50 | 0.50 | 0.50 | 0.50 | 0.50 | 0.50 | 0.50 | 0.50 | 0.50 |
| | Ether/Acrylates Copolymer | | | | | | | | | |

| | | | | | | | | | |
|---|---|---|---|---|---|---|---|---|---|
| In vitro SPF measured according the | 44.3 | 34.5 | 33.7 | 57.1 | 35.2 | 49 | 47.4 | 56.9 | 57.3 |
| method described | | | | | | | | | |
| UVA PF measured according the | 22 | 18 | 13.9 | 21.5 | 13.6 | 16.3 | 14 | 15 | 12.8 |
| method described | | | | | | | | | |

### Manufacturing Instruction:

Part A is prepared by incorporating all ingredients, then stirred under moderate speed and heated to 75° C. Part C is prepared and heated to 75° C. At this temperature, part A is poured into part C under progressive stirring speed. Below 65° C. the ingredients of part B are added separately. After cooling down under moderate stirring to 40° C. part D is added.

### EXAMPLE B9: O/W Every Day UV Protection Lotion

Example B9: O/W Every Day UV Protection Lotion

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| Part A | Glyceryl Stearate (and) PEG- | 5.00 | 5.00 | 5.00 | 5.00 | 5.00 | 5.00 | 5.00 | 5.00 | 5.00 |
| | 100 Stearate | | | | | | | | | |
| | Stearyl Alcohol | 1.00 | 1.00 | 1.00 | 1.00 | 1.00 | 1.00 | 1.00 | 1.00 | 1.00 |
| | Tripalmitin | 0.70 | 0.70 | 0.70 | 0.70 | 0.70 | 0.70 | 0.70 | 0.70 | 0.70 |
| | Dimethicone | 2.00 | 2.00 | 2.00 | 2.00 | 2.00 | 2.00 | 2.00 | 2.00 | 2.00 |
| | C12-15 Alkyl Benzoate | 5.00 | 5.00 | 5.00 | 5.00 | 5.00 | 5.00 | 5.00 | 5.00 | 5.00 |
| | Isopropyl Palmitate | 5.00 | 5.00 | 5.00 | 5.00 | 5.00 | 5.00 | 5.00 | 5.00 | 5.00 |
| | Drometrizole trisiloxane | 10.00 | 8.00 | 7.00 | 5.00 | 4.00 | 3.00 | 2.00 | 1.50 | 1.00 |
| | Ethylhexyl Methoxycinnamate | 6.00 | 8.00 | 4.00 | 3.00 | 2.00 | 4.00 | 5.00 | 3.00 | 3.00 |
| | Titanium dioxide | 4.00 | 5.00 | 3.00 | 2.50 | 2.00 | 2.00 | 1.00 | 1.50 | 1.00 |
| Part B | Organic micro-particulate UV | 2.00 | 4.00 | 6.00 | 8.00 | 10.0 | 14.0 | 16.0 | 18.0 | 20.0 |
| | absorber of formula (3), (5) or | | | | | | | | | |
| | (7) | | | | | | | | | |
| Part C | Water | qs to | qs to | qs to | qs to | qs to | qs to | qs to | qs to | qs to |
| | | 100 | 100 | 100 | 100 | 100 | 100 | 100 | 100 | 100 |
| | Polysorbate 60 | 0.50 | 0.50 | 0.50 | 0.50 | 0.50 | 0.50 | 0.50 | 0.50 | 0.50 |
| | Glycerin | 3.00 | 3.00 | 3.00 | 3.00 | 3.00 | 3.00 | 3.00 | 3.00 | 3.00 |
| Part D | Phenoxyethanol (and) Methyl- | 0.70 | 0.70 | 0.70 | 0.70 | 0.70 | 0.70 | 0.70 | 0.70 | 0.70 |
| | paraben (and) Ethylparaben | | | | | | | | | |
| | (and) Butylparaben (and) Propyl- | | | | | | | | | |
| | paraben (and) Isobutylparaben | | | | | | | | | |
| | Steareth-10 Allyl | 1.50 | 1.50 | 1.50 | 1.50 | 1.50 | 1.50 | 1.50 | 1.50 | 1.50 |
| | Ether/Acrylates Copolymer | | | | | | | | | |
| Part E | Water (and) Sodium Hydroxide | qs | qs | qs | qs | qs | qs | qs | qs | qs |
| | Disodium Phenyl | 7.00 | 5.00 | 4.00 | 3.00 | 2.00 | 3.00 | 1.50 | 1.00 | 1.00 |
| | Dibenzimidazole Tetrasulfonate | | | | | | | | | |
| Part F | Fragrance | qs | qs | qs | qs | qs | qs | qs | qs | qs |

| | | | | | | | | | |
|---|---|---|---|---|---|---|---|---|---|
| In vitro SPF measured according the | 47.6 | 50.2 | 41.6 | 38.3 | 36 | 49.1 | 45.9 | 47.8 | 51.9 |
| method described | | | | | | | | | |
| UVA PF measured according the | 15.8 | 14.4 | 12.9 | 11.4 | 10.5 | 12.1 | 10.6 | 10.7 | 11.3 |
| method described | | | | | | | | | |

### Manufacturing Instruction:

Part A is prepared by incorporating all ingredients, then stirred under moderate speed and heated to 75° C. Part C is prepared and heated to 75° C. At this temperature, part A is poured into part C under progressive stirring speed. Below 65° C. the ingredients of part B are added separately. After cooling down under moderate stirring to 40° C. part E and D are added. Then, incorporate fragrance and check the pH.

### EXAMPLE B10: O/W Sprayable Lotion

Example B10: O/W Sprayable Lotion

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| Part A | Cetearyl Alcohol (and) | 3.50 | 3.50 | 3.50 | 3.50 | 3.50 | 3.50 | 3.50 | 3.50 | 3.50 |
| | Polysorbate Oil | | | | | | | | | |
| | PEG-40 Hydrogenated Castor Oil | 4.00 | 4.00 | 4.00 | 4.00 | 4.00 | 4.00 | 4.00 | 4.00 | 4.00 |
| | Almond oil seet (prunus dulcis) | 1.00 | 1.00 | 1.00 | 1.00 | 1.00 | 1.00 | 1.00 | 1.00 | 1.00 |
| | Glyceryl Stearate | 1.00 | 1.00 | 1.00 | 1.00 | 1.00 | 1.00 | 1.00 | 1.00 | 1.00 |
| | PEG-7 Glyceryl Cocoate | 2.00 | 2.00 | 2.00 | 2.00 | 2.00 | 2.00 | 2.00 | 2.00 | 2.00 |
| | Isopropyl Palmitate | 4.00 | 4.00 | 4.00 | 4.00 | 4.00 | 4.00 | 4.00 | 4.00 | 4.00 |
| | Diethylhexyl Butamido Triazone | 3.00 | 1.50 | 2.00 | 2.50 | 1.00 | 1.00 | 1.00 | 1.50 | 2.00 |
| | Octocrylene | 10.0 | 4.00 | 5.00 | 4.00 | 4.00 | 5.00 | 3.00 | 4.00 | 2.00 |
| | Bis-Ethylhexyloxyphenol | 4.00 | 3.00 | 2.00 | 6.00 | 2.50 | 3.00 | 1.50 | 2.00 | 1.00 |
| | Methoxyphenyl Triazine | | | | | | | | | |
| | Diethylamino Hydroxybenzoyl | 5.00 | 3.00 | 2.00 | 2.50 | 3.00 | 1.00 | 2.00 | 3.00 | 1.50 |
| | hexyl benzoate | | | | | | | | | |
| Part B | Organic micro-particulate UV absorber | 2.00 | 4.00 | 6.00 | 8.00 | 10.0 | 14.0 | 16.0 | 18.0 | 20.0 |
| | of formula (3), (5) or (7) | | | | | | | | | |
| Part C | Water | Qs | Qs | Qs | Qs | Qs | Qs | Qs | Qs | Qs |
| | | to | to | to | to | to | to | to | to | to |
| | | 100 | 100 | 100 | 100 | 100 | 100 | 100 | 100 | 100 |
| | Glycerine | 2.00 | 2.00 | 2.00 | 2.00 | 2.00 | 2.00 | 2.00 | 2.00 | 2.00 |
| | Propylene glycol | 2.50 | 2.50 | 2.50 | 2.50 | 2.50 | 2.50 | 2.50 | 2.50 | 2.50 |
| | Terephthalidene Dicamphor | 8.00 | 6.00 | 4.00 | 3.00 | 2.00 | 2.00 | 1.00 | 1.50 | 1.00 |
| | sulfonic acid | | | | | | | | | |
| Part D | Preservative (Nipa) | q.s. | q.s. | q.s. | q.s. | q.s. | q.s. | q.s. | q.s. | q.s. |
| | Perfume Oil (Luzi) | q.s. | q.s. | q.s. | q.s. | q.s. | q.s. | q.s. | q.s. | q.s. |

| | | | | | | | | | |
|---|---|---|---|---|---|---|---|---|---|
| In vitro SPF measured according the | 73.6 | 39.2 | 31.9 | 54.8 | 25.7 | 28.5 | 18.1 | 28.4 | 21.7 |
| method described | | | | | | | | | |
| UVA PF measured according the | 38 | 27.5 | 20.8 | 28.2 | 20.8 | 19.2 | 16 | 21.2 | 15.1 |
| method described | | | | | | | | | |

### Manufacturing Instruction:

Part A is prepared by incorporating all ingredients, then stirred under moderate speed and heated to 75° C. Part C is prepared and heated to 75° C. At this temperature, part A is poured into part C under progressive stirring speed. Below 65° C. the ingredients of part B are added separately. After cooling down under moderate stirring to 40° C. part D is added.

### EXAMPLE B11: Cold Process Spray

Example B11: Cold Process Spray

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| Part A | Sorbitan-30 | 3.00 | 3.00 | 3.00 | 3.00 | 3.00 | 3.00 | 3.00 | 3.00 | 3.00 |
| Part B | Isostearyl Alcohol | 6.00 | 6.00 | 6.00 | 6.00 | 6.00 | 6.00 | 6.00 | 6.00 | 6.00 |
| | Sorbitan Oleate (and) Polyglycerol | 2.00 | 2.00 | 2.00 | 2.00 | 2.00 | 2.00 | 2.00 | 2.00 | 2.00 |
| | 3-Polyricinoleate | | | | | | | | | |
| | Polysorbate-20 | 1.00 | 1.00 | 1.00 | 1.00 | 1.00 | 1.00 | 1.00 | 1.00 | 1.00 |
| | Dimethicone | 1.00 | 1.00 | 1.00 | 1.00 | 1.00 | 1.00 | 1.00 | 1.00 | 1.00 |
| | Octocrylene | 10.00 | 4.00 | 5.00 | 4.00 | 4.00 | 5.00 | 3.00 | 4.00 | 2.00 |
| | Bis-Ethylhexyloxyphenol | 4.00 | 3.00 | 2.00 | 6.00 | 2.50 | 3.00 | 1.50 | 2.00 | 1.00 |
| | Methoxyphenyl Triazine | | | | | | | | | |
| | Titanium dioxide | 4.00 | 5.00 | 3.00 | 2.50 | 2.00 | 2.00 | 1.00 | 1.50 | 1.00 |
| Part C | Organic micro-particulate UV | 2.00 | 4.00 | 6.00 | 8.00 | 10.0 | 14.0 | 16.0 | 18.0 | 20.0 |
| | absorber of formula (3), (5) or | | | | | | | | | |
| | (7) | | | | | | | | | |
| Part D | DMDM Hydantoin | 1.00 | 1.00 | 1.00 | 1.00 | 1.00 | 1.00 | 1.00 | 1.00 | 1.00 |
| | Aqua | Qs | Qs | Qs | Qs | Qs | Qs | Qs | Qs | Qs to |
| | | to | to | to | to | to | to | to | to | 100 |
| | | 100 | 100 | 100 | 100 | 100 | 100 | 100 | 100 | |
| | Hydroxypropyl Starch | 4.00 | 4.00 | 4.00 | 4.00 | 4.00 | 4.00 | 4.00 | 4.00 | 4.00 |
| | Phosphate | | | | | | | | | |
| | Acrylates Copolymer | 3.35 | 3.35 | 3.35 | 3.35 | 3.35 | 3.35 | 3.35 | 3.35 | 3.35 |
| | Citric Acid (and) Silver Citrate | 0.10 | 0.10 | 0.10 | 0.10 | 0.10 | 0.10 | 0.10 | 0.10 | 0.10 |
| | Sclerotium Gum | 0.10 | 0.10 | 0.10 | 0.10 | 0.10 | 0.10 | 0.10 | 0.10 | 0.10 |

| | | | | | | | | | |
|---|---|---|---|---|---|---|---|---|---|
| In vitro SPF measured according the | 35.4 | 27.1 | 19.1 | 31.9 | 17.8 | 22.3 | 11.8 | 16.5 | 10.2 |
| method described | | | | | | | | | |
| UVA PF measured according the | 11.9 | 11.5 | 9.6 | 16.3 | 10.9 | 13.1 | 10.3 | 12.1 | 10.3 |
| method described | | | | | | | | | |

### EXAMPLE B11: Cold Process Spray

### Manufacturing Instruction:

Part A is prepared by incorporating all ingredients, then stirred under moderate speed. Part D is prepared. At room temperature, part A is poured into part D under progressive stirring speed. Below 65° C. the ingredients of part C are added separately. Then the pH is checked and adjusted.

### EXAMPLE B12: Sun Spray Foaming

Example B12: Sun spray foaming

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| Part A | Behenyl Alcohol (and) Glyceryl | 0.50 | 0.50 | 0.50 | 0.50 | 0.50 | 0.50 | 0.50 | 0.50 | 0.50 |
| | Stearate (and) Glyceryl | | | | | | | | | |
| | Stearate Citrate (and) Disodium | | | | | | | | | |
| | Ethylene Di(Cocamide PEG-15 | | | | | | | | | |
| | Disulfate) | | | | | | | | | |
| | Isotrideceth-12 | 0.50 | 0.50 | 0.50 | 0.50 | 0.50 | 0.50 | 0.50 | 0.50 | 0.50 |
| | Ethylhexyl Salicylate | 4.00 | 4.00 | 4.00 | 4.00 | 4.00 | 4.00 | 4.00 | 4.00 | 4.00 |
| | Hydrogenated Coco-glycerides | 1.50 | 1.50 | 1.50 | 1.50 | 1.50 | 1.50 | 1.50 | 1.50 | 1.50 |
| | C12-15 Alkyl Benzoate | 8.00 | 8.00 | 8.00 | 8.00 | 8.00 | 8.00 | 8.00 | 8.00 | 8.00 |
| | Ditehylhexyl butamido Triazone | 3.00 | 1.50 | 2.00 | 2.50 | 1.00 | 1.00 | 1.00 | 1.50 | 2.00 |
| | Bis-Ethylhexyloxyphenol | 4.00 | 3.00 | 2.00 | 6.00 | 2.50 | 3.00 | 1.50 | 2.00 | 1.00 |
| | Methoxyphenyl Triazine | | | | | | | | | |
| | Octocrylene | 10.0 | 4.00 | 5.00 | 4.00 | 4.00 | 5.00 | 3.00 | 4.00 | 2.00 |
| | Butyl Methoxy Dibenzoyl | 3.00 | 2.00 | 2.50 | 3.00 | 2.00 | 1.50 | 1.50 | 1.00 | 1.00 |
| | Methane | | | | | | | | | |
| Part B | Organic micro-particulate UV | 2.00 | 4.00 | 6.00 | 8.00 | 10.0 | 14.0 | 16.0 | 18.0 | 20.0 |
| | absorber of formula (3), (5) or | | | | | | | | | |
| | (7) | | | | | | | | | |
| Part C | Aqua | 5.00 | 5.00 | 5.00 | 5.00 | 5.00 | 5.00 | 5.00 | 5.00 | 5.00 |
| | Glycerin | 4.00 | 4.00 | 4.00 | 4.00 | 4.00 | 4.00 | 4.00 | 4.00 | 4.00 |
| | Galactoarabinan | 0.25 | 0.25 | 0.25 | 0.25 | 0.25 | 0.25 | 0.25 | 0.25 | 0.25 |
| Part D | Disodium Ethylene | 1.00 | 1.00 | 1.00 | 1.00 | 1.00 | 1.00 | 1.00 | 1.00 | 1.00 |
| | Di(Cocamide PEG-15 Disulfate) | | | | | | | | | |
| | (and) Sodium Lauroyl Lactylate | | | | | | | | | |
| | Water | Qs | Qs | Qs | Qs | Qs | Qs | Qs | Qs | Qs |
| | | to | to | to | to | to | to | to | to | to |
| | | 100 | 100 | 100 | 100 | 100 | 100 | 100 | 100 | 100 |
| | Zinc Oxide | 4.00 | 5.00 | 3.00 | 2.50 | 2.00 | 2.00 | 1.00 | 1.50 | 1.00 |
| Part E | Phenoxyethanol (and) Methyl- | 0.70 | 0.70 | 0.70 | 0.70 | 0.70 | 0.70 | 0.70 | 0.70 | 0.70 |
| | paraben (and) Ethylparaben | | | | | | | | | |
| | (and) Butylparaben (and) Propylparaben | | | | | | | | | |
| | (and) | | | | | | | | | |
| | Isobutylparaben | | | | | | | | | |
| | Tocopheryl Acetate | 0.60 | 0.60 | 0.60 | 0.60 | 0.60 | 0.60 | 0.60 | 0.60 | 0.60 |

| | | | | | | | | | |
|---|---|---|---|---|---|---|---|---|---|
| In vitro SPF measured according the | 58.8 | 27.6 | 28.2 | 53.7 | 21.4 | 25.7 | 16.4 | 23.1 | 19.3 |
| method described | | | | | | | | | |
| UVA PF measured according the | 19.4 | 14.9 | 13.4 | 22.8 | 14.3 | 16.2 | 12.6 | 14 | 11.8 |
| method described | | | | | | | | | |

### EXAMPLE B12: Sun Spray Foaming

### Manufacturing Instruction:

Part A is prepared by incorporating all ingredients, then stirred under moderate speed and heated to 75° C. Part C is prepared and heated to 75° C. At this temperature, part A is poured into part C under progressive stirring speed. Immediately after homogenization of the mixture, part D is poured under stirring. Below 65° C. the ingredients of part B are added separately. After cooling down under moderate stirring to 40° C. part E is added . Then, check the pH.

### EXAMPLE B13: O/W Sunscreen Cream

Example B13: O/W Sunscreen Cream
Foameous

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| Part A | Stearic Acid | 1.00 | 1.00 | 1.00 | 1.00 | 1.00 | 1.00 | 1.00 | 1.00 | 1.00 |
| | Cetearyl Alcohol | 2.00 | 2.00 | 2.00 | 2.00 | 2.00 | 2.00 | 2.00 | 2.00 | 2.00 |
| | PEG-20-Stearate | 1.00 | 1.00 | 1.00 | 1.00 | 1.00 | 1.00 | 1.00 | 1.00 | 1.00 |
| | Caprylic Acid/Caprinic Acid | 2.00 | 2.00 | 2.00 | 2.00 | 2.00 | 2.00 | 2.00 | 2.00 | 2.00 |
| | Triglyceride | | | | | | | | | |
| | Paraffin Oil | 10.00 | 10.00 | 10.00 | 10.00 | 10.00 | 10.00 | 10.00 | 10.00 | 10.00 |
| | Cyclomethicone | 5.0 | 5.0 | 5.0 | 5.0 | 5.0 | 5.0 | 5.0 | 5.0 | 5.0 |
| | Dimethicone | 0.5 | 0.5 | 0.5 | 0.5 | 0.5 | 0.5 | 0.5 | 0.5 | 0. |
| | Drometrizole Trisiloxane | 10.0 | 8.00 | 7.00 | 5.00 | 4.00 | 3.00 | 2.00 | 1.50 | 1.00 |
| | Polysilicone-15 | 6.0 | 8.0 | 4.0 | 3.0 | 2.0 | 4.0 | 5.0 | 3. | 3.0 |
| | Bis-Ethylhexyloxyphenol | 4.0 | 3.0 | 2.0 | 6.0 | 2.5 | 3.0 | 1.5 | 2.0 | 1.0 |
| | Methoxyphenyl Triazine | | | | | | | | | |
| | Dimethicone/Vinyl | 1.5 | 1.5 | 1.5 | 1.5 | 1.5 | 1.5 | 1.5 | 1.5 | 1.5 |
| | Dimethicone Crosspolymer | | | | | | | | | |
| | Octylisostearate | 2.00 | 2.00 | 2.00 | 2.00 | 2.00 | 2.00 | 2.00 | 2.00 | 2.00 |
| | Myristyl Myristate | 1.0 | 1.0 | 1.0 | 1.0 | 1.0 | 1.0 | 1.0 | 1.0 | 1.0 |
| Part B | Glycerine | 6.0 | 6.0 | 6.0 | 6.0 | 6.0 | 6.0 | 6.0 | 6.0 | 6.0 |
| | Carboxymethylcellulose | 0.05 | 0.05 | 0.05 | 0.05 | 0.05 | 0.05 | 0.05 | 0.05 | 0.05 |
| | Magnesium Aluminium | 0.50 | 0.50 | 0.50 | 0.50 | 0.50 | 0.50 | 0.50 | 0.50 | 0.50 |
| | Silicate | | | | | | | | | |
| | PEG-180/Laureth-50/TMMG | 0.50 | 0.50 | 0.50 | 0.50 | 0.50 | 0.50 | 0.50 | 0.50 | 0.50 |
| | Copolymer | | | | | | | | | |
| | Talcum | 0.50 | 0.50 | 0.50 | 0.50 | 0.50 | 0.50 | 0.50 | 0.50 | 0.50 |
| | BHT | 0.02 | 0.02 | 0.02 | 0.02 | 0.02 | 0.02 | 0.02 | 0.02 | 0.02 |
| | Phenylbenzimidazole sulfonic | 7.0 | 5.0 | 4.0 | 3.0 | 2.0 | 3.0 | 1.5 | 1.0 | 1.0 |
| | acid | | | | | | | | | |
| | Disodium EDTA | 0.10 | 0.10 | 0.10 | 0.10 | 0.10 | 0.10 | 0.10 | 0.10 | 0.10 |
| | KOH | q.s | q.s | q.s | q.s | q.s | q.s | q.s | q.s | q.s |
| | Water | Qs to | Qs to | Qs to | Qs to | Qs to | Qs to | Qs to | Qs to | Qs to |
| | | 100 | 100 | 100 | 100 | 100 | 100 | 100 | 100 | 100 |
| Part C | Organic micro-particulate UV | 2.0 | 4.0 | 6.0 | 8.0 | 10. | 14.0 | 16.0 | 18.0 | 20.0 |
| | absorber of formula (3), (5) or | | | | | | | | | |
| | (7) | | | | | | | | | |
| Part E | Parfum, Preservative, Dyes | q.s. | q.s. | q.s. | q.s. | q.s. | q.s. | q.s. | q.s. | q.s. |

| | | | | | | | | | |
|---|---|---|---|---|---|---|---|---|---|
| In vitro SPF measured according | 55.9 | 45.8 | 35.4 | 43.5 | 23.2 | 31.8 | 18 | 15.5 | 12.5 |
| the method described | | | | | | | | | |
| UVA PF measured according the | 12.8 | 11.4 | 10.2 | 16.5 | 11 | 12.6 | 10.3 | 11.3 | 9.9 |
| method described | | | | | | | | | |

### Manufacturing Instruction:

Part A is prepared by incorporating all ingredients, then stirred under moderate speed and heated to 75° C. Part B is prepared and heated to 75° C. At this temperature, part A is poured into part B under progressive stirring speed. Below 65° C. the ingredient of part C is added separately. After cooling down under moderate stirring to 40° C. part E is added . Then, check the pH.

### EXAMPLE B14: Continuous Spray Fresh Cooling

Example B14: Continuous Spray Fresh Cooling

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| Part A | SD-Alcohol 40 | 58.00 | 58.00 | 58.00 | 58.00 | 58.00 | 58.00 | 58.00 | 58.00 | 58.00 |
| | Diethylhexyl 2,6- | 6.00 | 6.00 | 6.00 | 6.00 | 6.00 | 6.00 | 6.00 | 6.00 | 6.00 |
| | Naphthalate | | | | | | | | | |
| | Trisiloxane (and) | 4.00 | 4.00 | 4.00 | 4.00 | 4.00 | 4.00 | 4.00 | 4.00 | 4.00 |
| | Dimethicone | | | | | | | | | |
| | Acrylates/Octylacrylamide | 3.00 | 3.00 | 3.00 | 3.00 | 3.00 | 3.00 | 3.00 | 3.00 | 3.00 |
| | Copolymer | | | | | | | | | |
| | PPG-5-Ceteth-20 | 2.00 | 2.00 | 2.00 | 2.00 | 2.00 | 2.00 | 2.00 | 2.00 | 2.00 |
| | Tocopheryl Acetate | 1.00 | 1.00 | 1.00 | 1.00 | 1.00 | 1.00 | 1.00 | 1.00 | 1.00 |
| | Ascorbyl Palmitate | 0.3 | 0.3 | 0.3 | 0.3 | 0.3 | 0.3 | 0.3 | 0.3 | 0.3 |
| | Retinyl Palmitate | 0.3 | 0.3 | 0.3 | 0.3 | 0.3 | 0.3 | 0.3 | 0.3 | 0.3 |
| | Cyclopentasiloxane (and) | 1.00 | 1.00 | 1.00 | 1.00 | 1.00 | 1.00 | 1.00 | 1.00 | 1.00 |
| | Acrylates/Dimethicone | | | | | | | | | |
| | Copolymer | | | | | | | | | |
| | Diethylamino | 5.00 | 3.00 | 2.00 | 2.50 | 3.00 | 1.00 | 2.00 | 3.00 | 1.50 |
| | Hydroxybenzoyl hexyl | | | | | | | | | |
| | Benzoate | | | | | | | | | |
| | Ethylhexyl triazone | 3.00 | 1.50 | 2.00 | 2.50 | 1.00 | 1.00 | 1.00 | 1.50 | 2.00 |
| | Bis-Ethylhexyloxyphenol | 4.00 | 3.00 | 2.00 | 6.00 | 2.50 | 3.00 | 1.50 | 2.00 | 1.00 |
| | Methoxyphenyl Triazine | | | | | | | | | |
| | Propylene Glycol (and) | 1.50 | 1.50 | 1.50 | 1.50 | 1.50 | 1.50 | 1.50 | 1.50 | 1.50 |
| | Ethoxydiglycol (and) | | | | | | | | | |
| | Menthyl PCA (and) Lauryl | | | | | | | | | |
| | PCA | | | | | | | | | |
| Part C | Organic micro-particulate | 2.00 | 4.00 | 6.00 | 8.00 | 10.00 | 14.00 | 16.00 | 18.00 | 20.00 |
| | UV absorber of formula | | | | | | | | | |
| | (3), (5) or (7) | | | | | | | | | |
| Part D | Phenylbenzimidazole | 7.00 | 5.00 | 4.00 | 3.00 | 2.00 | 3.00 | 1.50 | 1.00 | 1.00 |
| | sulfonic acid | | | | | | | | | |
| | Parfume | q.s. | q.s. | q.s. | q.s. | q.s. | q.s. | q.s. | q.s. | q.s. |
| | Aqua | Qs to | Qs to | Qs to | Qs to | Qs to | Qs to | Qs to | Qs to | Qs to |
| | | 100 | 100 | 100 | 100 | 100 | 100 | 100 | 100 | 100 |
| Part B | Propellent | Qs | Qs | Qs | Qs | Qs | Qs | Qs | Qs | Qs |

| | | | | | | | | | |
|---|---|---|---|---|---|---|---|---|---|
| In vitro SPF measured | 60.5 | 40.3 | 34.9 | 51.4 | 24.8 | 31.1 | 19.1 | 22.4 | 20.8 |
| according the method described | | | | | | | | | |
| UVA PF measured according | 17.1 | 12.4 | 9.9 | 18.6 | 13.9 | 12.6 | 12.3 | 15.5 | 11.7 |
| the method described | | | | | | | | | |

### Manufacturing Instruction:

Part A is prepared by incorporating all ingredients, then stirred under moderate speed .Part D is prepared. Part A is poured into part D under progressive stirring speed. Immediately after homogenization of the mixture, part C is poured under stirring. Part B is added to the mixture by specific process for continuous spray system.

### EXAMPLE B15: PEG-free Sunscreen

Example B15: PEG-free sunscreen

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| Part A | Hexyldecanol | 2.30 | 2.30 | 2.30 | 2.30 | 2.30 | 2.30 | 2.30 | 2.30 | 2.30 |
| | Polyglyceryl-3 Methylglucose | 1.40 | 1.40 | 1.40 | 1.40 | 1.40 | 1.40 | 1.40 | 1.40 | 1.40 |
| | Distearate | | | | | | | | | |
| | Polyglyceryl polyhydroxy | 1.40 | 1.40 | 1.40 | 1.40 | 1.40 | 1.40 | 1.40 | 1.40 | 1.40 |
| | stearate | | | | | | | | | |
| | Cetyl Ethylhexanoate | 3.00 | 3.00 | 3.00 | 3.00 | 3.00 | 3.00 | 3.00 | 3.00 | 3.00 |
| | Isohexadecane | 3.00 | 3.00 | 3.00 | 3.00 | 3.00 | 3.00 | 3.00 | 3.00 | 3.00 |
| | Octocrylene | 10.0 | 4.00 | 5.00 | 4.00 | 4.00 | 5.00 | 3.00 | 4.00 | 2.00 |
| | Bis-Ethylhexyloxyphenol | 4.00 | 3.00 | 2.00 | 6.00 | 2.50 | 3.00 | 1.50 | 2.00 | 1.00 |
| | Methoxyphenyl Triazine | | | | | | | | | |
| | Ethylhexyl Methoxycinnamate | 6.00 | 8.00 | 4.00 | 3.00 | 2.00 | 4.00 | 5.00 | 3.00 | 3.00 |
| | Butyl | 3.00 | 2.00 | 2.50 | 3.00 | 2.00 | 1.50 | 1.50 | 1.00 | 1.00 |
| | MethoxyDibenzoylMethane | | | | | | | | | |
| | Titanium Dioxide | 4.00 | 5.00 | 3.00 | 2.50 | 2.00 | 2.00 | 1.00 | 1.50 | 1.00 |
| | Phenoxyethanol (and) Methyl- | 0.60 | 0.60 | 0.60 | 0.60 | 0.60 | 0.60 | 0.60 | 0.60 | 0.60 |
| | paraben (and) Ethylparaben | | | | | | | | | |
| | (and) Butylparaben (and) Propylparaben | | | | | | | | | |
| | (and) Isobutyl- | | | | | | | | | |
| | paraben | | | | | | | | | |
| Part B | Organic micro-particulate UV | 2.00 | 4.00 | 6.00 | 8.00 | 10.0 | 14.0 | 16.0 | 18.0 | 20.0 |
| | absorber of formula (3), (5) or | | | | | | | | | |
| | (7) | | | | | | | | | |
| Part C | Water | Qs to | Qs to | Qs to | Qs to | Qs to | Qs to | Qs to | Qs to | Qs to |
| | | 100 | 100 | 100 | 100 | 100 | 100 | 100 | 100 | 100 |
| | Phenylbenzimidazole sulfonic | 7.00 | 5.00 | 4.00 | 3.00 | 2.00 | 3.00 | 1.50 | 1.00 | 1.00 |
| | acid | | | | | | | | | |
| | Glycerin | 3.00 | 3.00 | 3.00 | 3.00 | 3.00 | 3.00 | 3.00 | 3.00 | 3.00 |
| Part D | Sodium Acrylates Copolymer | 1.50 | 1.50 | 1.50 | 1.50 | 1.50 | 1.50 | 1.50 | 1.50 | 1.50 |
| | (and) Mineral Oil (and) PPG-1 | | | | | | | | | |
| | Trideceth-6 | | | | | | | | | |
| | Cyclopentasiloxane | 1.50 | 1.50 | 1.50 | 1.50 | 1.50 | 1.50 | 1.50 | 1.50 | 1.50 |

| | | | | | | | | | |
|---|---|---|---|---|---|---|---|---|---|
| In vitro SPF measured according the | 74.6 | 58.3 | 45.2 | 56.9 | 35.3 | 44 | 30.8 | 33.9 | 23.5 |
| method described | | | | | | | | | |
| UVA PF measured according the | 15.3 | 13.1 | 11.4 | 19.1 | 13.5 | 15.2 | 11.6 | 13.8 | 11.5 |
| method described | | | | | | | | | |

### Manufacturing Instruction:

Part A is prepared by incorporating all ingredients, then stirred under moderate speed. Part C is prepared. At room temperature, part A is poured into part C under progressive stirring speed. Part D is incorporated into the previously obtained mixture, under moderate stirring. Then the ingredient of part B is added separately. Then the pH is checked and adjusted.

### EXAMPLE B16: Skin Protection Sunscreen W/O

Example B16: Skin Proteciton Sunscreen W/O

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| Part A | Glyceryl Oleate | 4.50 | 4.50 | 4.50 | 4.50 | 4.50 | 4.50 | 4.50 | 4.50 | 4.50 |
| | PEG-7 Hydrogenated Castor Oil | 1.50 | 1.50 | 1.50 | 1.50 | 1.50 | 1.50 | 1.50 | 1.50 | 1.50 |
| | Hydrogenated Castor Oil | 0.50 | 0.50 | 0.50 | 0.50 | 0.50 | 0.50 | 0.50 | 0.50 | 0.50 |
| | Microcrystalline Wax | 2.00 | 2.00 | 2.00 | 2.00 | 2.00 | 2.00 | 2.00 | 2.00 | 2.00 |
| | Beeswax | 1.50 | 1.50 | 1.50 | 1.50 | 1.50 | 1.50 | 1.50 | 1.50 | 1.50 |
| | C12-15 Alkyl Benzoate | 3.00 | 3.00 | 3.00 | 3.00 | 3.00 | 3.00 | 3.00 | 3.00 | 3.00 |
| | Isopropyl Isostearate | 2.50 | 2.50 | 2.50 | 2.50 | 2.50 | 2.50 | 2.50 | 2.50 | 2.50 |
| | Mineral Oil | 5.00 | 5.00 | 5.00 | 5.00 | 5.00 | 5.00 | 5.00 | 5.00 | 5.00 |
| | Diethylamino hydroxybenzoyl | 5.00 | 3.00 | 2.00 | 2.50 | 3.00 | 1.00 | 2.00 | 3.00 | 1.50 |
| | hexyl Benzoate | | | | | | | | | |
| | Bis-Ethylhexyloxyphenol | 4.00 | 3.00 | 2.00 | 6.00 | 2.50 | 3.00 | 1.50 | 2.00 | 1.00 |
| | Methoxyphenyl Triazine | | | | | | | | | |
| | Ethylhexyl Methoxycinnamate | 6.00 | 8.00 | 4.00 | 3.00 | 2.00 | 4.00 | 5.00 | 3.00 | 3.00 |
| | Ethylhexyl triazone | 3.00 | 1.50 | 2.00 | 2.50 | 1.00 | 1.00 | 1.00 | 1.50 | 2.00 |
| | Drometrizole trisiloxane | 10.00 | 8.00 | 7.00 | 5.00 | 4.00 | 3.00 | 2.00 | 1.50 | 1.00 |
| Part B | Organic micro-particulate UV | 2.00 | 4.00 | 6.00 | 8.00 | 10.0 | 14.0 | 16.0 | 18.0 | 20.0 |
| | absorber of formula (3), (5) or | | | | | | | | | |
| | (7) | | | | | | | | | |
| Part C | Aqua | qs to | qs to | qs to | qs to | qs to | qs to | qs to | qs to | qs to |
| | | 100 | 100 | 100 | 100 | 100 | 100 | 100 | 100 | 100 |
| | Magnesium Sulfate | 0.50 | 0.50 | 0.50 | 0.50 | 0.50 | 0.50 | 0.50 | 0.50 | 0.50 |
| | Terephthalidene dicamphor | 8.00 | 6.00 | 4.00 | 3.00 | 2.00 | 2.00 | 1.00 | 1.50 | 1.00 |
| | sulfonic acid | | | | | | | | | |
| Part D | Citric Acid | 0.05 | 0.05 | 0.05 | 0.05 | 0.05 | 0.05 | 0.05 | 0.05 | 0.05 |
| | Citric Acid (and) Silver Citrate | 0.10 | 0.10 | 0.10 | 0.10 | 0.10 | 0.10 | 0.10 | 0.10 | 0.10 |
| | Sclerotium Gum | 0.10 | 0.10 | 0.10 | 0.10 | 0.10 | 0.10 | 0.10 | 0.10 | 0.10 |

| | | | | | | | | | |
|---|---|---|---|---|---|---|---|---|---|
| In vitro SPF measured according the | 82.3 | 59.6 | 45.5 | 64.6 | 29.9 | 33.8 | 26.9 | 29.5 | 24.7 |
| method described | | | | | | | | | |
| UVA PF measured according the | 43.9 | 32.6 | 24.7 | 30.9 | 22.7 | 20.4 | 16.9 | 20.7 | 14.5 |
| method described | | | | | | | | | |

### Manufacturing Instruction:

Part A is prepared by incorporating all ingredients, then stirred under moderate speed and heated to 75° C. Part C is prepared and heated to 75° C. At this temperature, part C is poured into part A under progressive stirring speed. Below 65° C. the ingredient of part B is added separately. After cooling down under moderate stirring to 40° C. part D is added . Then, check the pH.

### EXAMPLE B17: W/Si Sun Cream

Example B17: W/ Si sun cream

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| Part A | Cyclopentasiloxane (and) | 10.0 | 10.0 | 10.0 | 10.0 | 10.0 | 10.0 | 10.0 | 10.0 | 10.0 |
| | PEG/PPG-18/18 Dimethicone | | | | | | | | | |
| | Cyclomethicone | 9.00 | 9.00 | 9.00 | 9.00 | 9.00 | 9.00 | 9.00 | 9.00 | 9.00 |
| | Cyclomethicone (and) | 3.50 | 3.50 | 3.50 | 3.50 | 3.50 | 3.50 | 3.50 | 3.50 | 3.50 |
| | Trimethylsiloxysilicate | | | | | | | | | |
| | Cyclopentasiloxane (and) | 7.00 | 7.00 | 7.00 | 7.00 | 7.00 | 7.00 | 7.00 | 7.00 | 7.00 |
| | Dimethicone Crosspolymer | | | | | | | | | |
| | Polysilicone-15 | 6.00 | 8.00 | 4.00 | 3.00 | 2.00 | 4.00 | 5.00 | 3.00 | 3.00 |
| | Diethylhexyl Butamido | 3.00 | 1.50 | 2.00 | 2.50 | 1.00 | 1.00 | 1.00 | 1.50 | 2.00 |
| | Triazone | | | | | | | | | |
| | Titanium Dioxide | 4.00 | 5.00 | 3.00 | 2.50 | 2.00 | 2.00 | 1.00 | 1.50 | 1.00 |
| Part B | Organic micro-particulate UV | 2.00 | 4.00 | 6.00 | 8.00 | 10.0 | 14.0 | 16.0 | 18.0 | 20.0 |
| | absorber of formula (3), (5) or | | | | | | | | | |
| | (7) | | | | | | | | | |
| Part C | Divinyldimethicone/Dimethicone | 2.30 | 2.30 | 2.30 | 2.30 | 2.30 | 2.30 | 2.30 | 2.30 | 2.30 |
| | Copolymer (and) C12-13 | | | | | | | | | |
| | Pareth-23 (and) C12-13 | | | | | | | | | |
| | Pareth-3 | | | | | | | | | |
| | Sodium Chloride | 2.00 | 2.00 | 2.00 | 2.00 | 2.00 | 2.00 | 2.00 | 2.00 | 2.00 |
| | Aqua | Qs to | Qs to | Qs to | Qs to | Qs to | Qs to | Qs to | Qs to | Qs to |
| | | 100 | 100 | 100 | 100 | 100 | 100 | 100 | 100 | 100 |

| | | | | | | | | | |
|---|---|---|---|---|---|---|---|---|---|
| In vitro SPF measured according the | 22.6 | 22.3 | 17.9 | 19.2 | 11.4 | 13.5 | 11.8 | 14.9 | 16.4 |
| method described | | | | | | | | | |
| UVA PF measured according the | 4.8 | 6.3 | 6.2 | 6.5 | 6.7 | 7.8 | 7.6 | 8.4 | 8.5 |
| method described | | | | | | | | | |

### Manufacturing Instruction:

Part A is prepared by incorporating all ingredients, then stirred under moderate speed. Part C is prepared and heated to 60° C. At this temperature, part C is poured into part A under progressive stirring speed. Then the ingredient of part B is added separately.

### EXAMPLE B18: W/ Si Emulsion

Example B18: W/ Si emulsion

| | | | B | C | D | E | F | G | H | I |
|---|---|---|---|---|---|---|---|---|---|---|
| | | A | % | % | % | % | % | % | % | % |
| | INCI-Name | % w/w | w/w | w/w | w/w | w/w | w/w | w/w | w/w | w/w |
| Part A | Tetrabutoxypropyl Trisiloxane | 0.90 | 0.90 | 0.90 | 0.90 | 0.90 | 0.90 | 0.90 | 0.90 | 0.90 |
| | Benzyl Laurate | 2.00 | 2.00 | 2.00 | 2.00 | 2.00 | 2.00 | 2.00 | 2.00 | 2.00 |
| | Cetyl dimethicone | 2.00 | 2.00 | 2.00 | 2.00 | 2.00 | 2.00 | 2.00 | 2.00 | 2.00 |
| | Polyglyceryl-4, isostearate, | 6.00 | 6.00 | 6.00 | 6.00 | 6.00 | 6.00 | 6.00 | 6.00 | 6.00 |
| | cetyl dimethicone copolyol and | | | | | | | | | |
| | hexyl laurate | | | | | | | | | |
| | polyglyceryl-3 dioleate | 0.60 | 0.60 | 0.60 | 0.60 | 0.60 | 0.60 | 0.60 | 0.60 | 0.60 |
| | Glyceryl tribehenate | 3.00 | 3.00 | 3.00 | 3.00 | 3.00 | 3.00 | 3.00 | 3.00 | 3.00 |
| | cyclomethicone | 8.85 | 8.85 | 8.85 | 8.85 | 8.85 | 8.85 | 8.85 | 8.85 | 8.85 |
| | Ethylhexyl Methoxycinnamate | 6.00 | 8.00 | 4.00 | 3.00 | 2.00 | 4.00 | 5.00 | 3.00 | 3.00 |
| | Bis-Ethylhexyloxyphenol | 4.00 | 3.00 | 2.00 | 6.00 | 2.50 | 3.00 | 1.50 | 2.00 | 1.00 |
| | Methoxyphenyl Triazine | | | | | | | | | |
| | Titanium Dioxide | 4.00 | 5.00 | 3.00 | 2.50 | 2.00 | 2.00 | 1.00 | 1.50 | 1.00 |
| Part B | Organic micro-particulate UV | 2.00 | 4.00 | 6.00 | 8.00 | 10.0 | 14.0 | 16.0 | 18.0 | 20.0 |
| | absorber of formula (3), (5) or | | | | | | | | | |
| | (7) | | | | | | | | | |
| Part C | Aqua | Qs to | Qs to | Qs to | Qs to | Qs to | Qs to | Qs to | Qs to | Qs to |
| | | 100 | 100 | 100 | 100 | 100 | 100 | 100 | 100 | 100 |
| | Xanthan gum | 0.10 | 0.10 | 0.10 | 0.10 | 0.10 | 0.10 | 0.10 | 0.10 | 0.10 |
| | Disodium EDTA | 0.10 | 0.10 | 0.10 | 0.10 | 0.10 | 0.10 | 0.10 | 0.10 | 0.10 |
| | Sodium chloride | 1.00 | 1.00 | 1.00 | 1.00 | 1.00 | 1.00 | 1.00 | 1.00 | 1.00 |
| | Glycerin | 3.00 | 3.00 | 3.00 | 3.00 | 3.00 | 3.00 | 3.00 | 3.00 | 3.00 |
| | Disodium Phenyl | 7.00 | 5.00 | 4.00 | 3.00 | 2.00 | 3.00 | 1.50 | 1.00 | 1.00 |
| | Dibenzimidazole tetrasulfonate | | | | | | | | | |
| | Terephthalidene Dicamphor | 8.00 | 6.00 | 4.00 | 3.00 | 2.00 | 2.00 | 1.00 | 1.50 | 1.00 |
| | sulfonic acid | | | | | | | | | |
| Part D | Citric Acid (and) Silver Citrate | 0.10 | 0.10 | 0.10 | 0.10 | 0.10 | 0.10 | 0.10 | 0.10 | 0.10 |
| | Sclerotium Gum | 0.10 | 0.10 | 0.10 | 0.10 | 0.10 | 0.10 | 0.10 | 0.10 | 0.10 |
| | panthenol | 0.10 | 0.10 | 0.10 | 0.10 | 0.10 | 0.10 | 0.10 | 0.10 | 0.10 |
| | Sodium ascorbyl phosphate | 0.20 | 0.20 | 0.20 | 0.20 | 0.20 | 0.20 | 0.20 | 0.20 | 0.20 |
| | tocopheryl acetate | 0.50 | 0.50 | 0.50 | 0.50 | 0.50 | 0.50 | 0.50 | 0.50 | 0.50 |
| | Phytantriol | 0.30 | 0.30 | 0.30 | 0.30 | 0.30 | 0.30 | 0.30 | 0.30 | 0.30 |
| | Fragrance/preservative | q.s. | q.s. | q.s. | q.s. | q.s. | q.s. | q.s. | q.s. | q.s. |

| | | | | | | | | | |
|---|---|---|---|---|---|---|---|---|---|
| In vitro SPF measured according the | 62.6 | 57.7 | 39.5 | 48.9 | 28.8 | 37.1 | 25.4 | 25.6 | 19.4 |
| method described | | | | | | | | | |
| UVA PF measured according the | 29.6 | 25.7 | 20.4 | 27.5 | 18.3 | 22 | 15.5 | 17.4 | 14.8 |
| method described | | | | | | | | | |

### Manufacturing Instruction:

Part A is prepared by incorporating all ingredients, then stirred under moderate speed. Part C is prepared and heated to 60° C. At this temperature, part C is poured into part A under progressive stirring speed. Then the ingredient of part B is added separately and the part E is added under moderate stirring.

### EXAMPLE B19: Sun Protection Gel (aqueous)

Example B19: sun Protection Gel (aqueous)

| | A | B | C | D | E | F | G | H | I |
|---|---|---|---|---|---|---|---|---|---|
| | % | % | % | % | % | % | % | % | % |
| INCI-Name | w/w | w/w | w/w | w/w | w/w | w/w | w/w | w/w | w/w |
| Allantoin | 0.20 | 0.20 | 0.20 | 0.20 | 0.20 | 0.20 | 0.20 | 0.20 | 0.20 |
| Sorbitol | 5.00 | 5.00 | 5.00 | 5.00 | 5.00 | 5.00 | 5.00 | 5.00 | 5.00 |
| Aqua | Qs to | Qs to | Qs to | Qs to | Qs to | Qs to | Qs to | Qs to | Qs to |
| | 100 | 100 | 100 | 100 | 100 | 100 | 100 | 100 | 100 |
| Disodium Phenyl Dibenzimidazole | 7.00 | 5.00 | 4.00 | 3.00 | 2.00 | 3.00 | 1.50 | 1.00 | 1.00 |
| tetrasulfonate | | | | | | | | | |
| Phenylbenzimidazole sulfonic | 8.00 | 6.00 | 4.00 | 3.00 | 2.00 | 2.00 | 1.00 | 1.50 | 1.00 |
| acid | | | | | | | | | |
| Zinc Oxide | 4.00 | 5.00 | 3.00 | 2.50 | 2.00 | 2.00 | 1.00 | 1.50 | 1.00 |
| Organic micro-particulate UV absorber | 2.00 | 4.00 | 6.00 | 8.00 | 10.0 | 14.0 | 16.0 | 18.0 | 20.0 |
| of formula (3), (5) or (7) | | | | | | | | | |
| Parfum | 0.30 | 0.30 | 0.30 | 0.30 | 0.30 | 0.30 | 0.30 | 0.30 | 0.30 |
| PEG-35-Hydrogenated Castor | 1.00 | 1.00 | 1.00 | 1.00 | 1.00 | 1.00 | 1.00 | 1.00 | 1.00 |
| Oil | | | | | | | | | |
| Carbomer | 1.50 | 1.50 | 1.50 | 1.50 | 1.50 | 1.50 | 1.50 | 1.50 | 1.50 |
| Aqua | 36.1 | 36.1 | 36.1 | 36.1 | 36.1 | 36.1 | 36.1 | 36.1 | 36.1 |
| Water, demineralized | 10.0 | 10.0 | 10.0 | 10.0 | 10.0 | 10.0 | 10.0 | 10.0 | 10.0 |
| Citric Acid (and) Silver Citrate | 0.10 | 0.10 | 0.10 | 0.10 | 0.10 | 0.10 | 0.10 | 0.10 | 0.10 |
| Sclerotium Gum | 0.10 | 0.10 | 0.10 | 0.10 | 0.10 | 0.10 | 0.10 | 0.10 | 0.10 |
| In vitro SPF measured according the | 35.5 | 34.6 | 31.4 | 30.8 | 29.5 | 41.8 | 38 | 44.2 | 47.1 |
| method described | | | | | | | | | |
| UVA PF measured according the | 20.8 | 19.3 | 15 | 13.4 | 11.7 | 14.1 | 11.3 | 12.9 | 12.3 |
| method described | | | | | | | | | |

All the ingredients are dispersed or solubilized inside the water phase under moderate stirring till an homogeneous gel appears. Then pH is checked.

### EXAMPLE B20: Foundations; O/W Forms

Example B20: Foundations; O/W forms

| | A | B | C | D | E | F | G | H | I |
|---|---|---|---|---|---|---|---|---|---|
| | % | % | % | % | % | % | % | % | % |
| INCI-Name | w/w | w/w | w/w | w/w | w/w | w/w | w/w | w/w | w/w |
| Butylene Glycol | 6.50 | 6.50 | 6.50 | 6.50 | 6.50 | 6.50 | 6.50 | 6.50 | 6.50 |
| Magnesium Aluminum Silicate | 0.50 | 0.50 | 0.50 | 0.50 | 0.50 | 0.50 | 0.50 | 0.50 | 0.50 |
| Sodium Carboxymethylcellulose | 0.30 | 0.30 | 0.30 | 0.30 | 0.30 | 0.30 | 0.30 | 0.30 | 0.30 |
| Xanthan Gum | 0.10 | 0.10 | 0.10 | 0.10 | 0.10 | 0.10 | 0.10 | 0.10 | 0.10 |
| Triethanolamine | 1.00 | 1.00 | 1.00 | 1.00 | 1.00 | 1.00 | 1.00 | 1.00 | 1.00 |
| Polysorbate 20 | 0.25 | 0.25 | 0.25 | 0.25 | 0.25 | 0.25 | 0.25 | 0.25 | 0.25 |
| Sericite | 7.00 | 7.00 | 7.00 | 7.00 | 7.00 | 7.00 | 7.00 | 7.00 | 7.00 |
| Iron oxides | 1.10 | 1.10 | 1.10 | 1.10 | 1.10 | 1.10 | 1.10 | 1.10 | 1.10 |
| Spherical Silica | 1.00 | 1.00 | 1.00 | 1.00 | 1.00 | 1.00 | 1.00 | 1.00 | 1.00 |
| Cetearyl octanoate | 10.0 | 10.0 | 10.0 | 10.0 | 10.0 | 10.0 | 10.0 | 10.0 | 10.0 |
| Stearic Acid | 2.50 | 2.50 | 2.50 | 2.50 | 2.50 | 2.50 | 2.50 | 2.50 | 2.50 |
| Glyceryl stearate | 2.00 | 2.00 | 2.00 | 2.00 | 2.00 | 2.00 | 2.00 | 2.00 | 2.00 |
| Tridecyl trimellilate | 2.00 | 2.00 | 2.00 | 2.00 | 2.00 | 2.00 | 2.00 | 2.00 | 2.00 |
| Bis-Ethylhexyloxyphenol | 4.00 | 3.00 | 2.00 | 6.00 | 2.50 | 3.00 | 1.50 | 2.00 | 1.00 |
| Methoxyphenyl Triazine | | | | | | | | | |
| Titanium Dioxide | 4.00 | 5.00 | 3.00 | 2.50 | 2.00 | 2.00 | 1.00 | 1.50 | 1.00 |
| Polysilicone-15 | 6.00 | 8.00 | 4.00 | 3.00 | 2.00 | 4.00 | 5.00 | 3.00 | 3.00 |
| Organic micro-particulate UV absorber | 2.00 | 4.00 | 6.00 | 8.00 | 10.00 | 14.00 | 16.0 | 18.0 | 20.0 |
| of formula (3), (5) or (7) | | | | | | | | | |
| Water | qs | qs | qs | qs | qs | qs | qs | qs | qs |
| | to 100 | to 100 | to 100 | to 100 | to 100 | to 100 | to 100 | to 100 | to 100 |
| Preservative | 0.55 | 0.55 | 0.55 | 0.55 | 0.55 | 0.55 | 0.55 | 0.55 | 0.55 |
| In vitro SPF measured according the | 24.5 | 26.6 | 14.8 | 27.6 | 13.4 | 17.3 | 11.1 | 13.1 | 9.6 |
| method described | | | | | | | | | |
| UVA PF measured according the | 11 | 11.2 | 9.1 | 15.8 | 10.5 | 12.5 | 9.9 | 11.6 | 10.1 |
| method described | | | | | | | | | |

### EXAMPLE B20: Foundations; O/W Forms

### EXAMPLE B21: Si/W Sun Cream

Example B21: Si/W sun cream

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| Part A | Cyclopentasiloxane | 8.00 | 8.00 | 8.00 | 8.00 | 8.00 | 8.00 | 8.00 | 8.00 | 8.00 |
| | (and) Dimethicone/Vinyl | | | | | | | | | |
| | Dimethicone | | | | | | | | | |
| | Crosspolymer | | | | | | | | | |
| | Dimethicone (and) Dimethicone/ | 30.00 | 30.00 | 30.00 | 30.00 | 30.00 | 30.00 | 30.00 | 30.00 | 30.00 |
| | Vinyl Dimethicone | | | | | | | | | |
| | Crosspolymer | | | | | | | | | |
| | Cyclopentasiloxane | 10.00 | 10.00 | 10.00 | 10.00 | 10.00 | 10.00 | 10.0 | 10.0 | 10.0 |
| | Polysiloxane-15 | 6.00 | 8.00 | 4.00 | 3.00 | 2.00 | 4.00 | 5.00 | 3.00 | 3.00 |
| | Drometrizole trisloxane | 10.00 | 8.00 | 7.00 | 5.00 | 4.00 | 3.00 | 2.00 | 1.50 | 1.00 |
| | Bis-Ethylhexyloxyphenol | 4.00 | 3.00 | 2.00 | 6.00 | 2.50 | 3.00 | 1.50 | 2.00 | 1.00 |
| | Methoxyphenyl Triazine | | | | | | | | | |
| | Ethylhexyl | 6.00 | 8.00 | 4.00 | 3.00 | 2.00 | 4.00 | 5.00 | 3.00 | 3.00 |
| | Methoxycinnamate | | | | | | | | | |
| Part B | 1.3-Butylen Glycol | 3.00 | 3.00 | 3.00 | 3.00 | 3.00 | 3.00 | 3.00 | 3.00 | 3.00 |
| | Polyglyceryl-3 Disiloxane | 0.60 | 0.60 | 0.60 | 0.60 | 0.60 | 0.60 | 0.60 | 0.60 | 0.60 |
| | Dimethicone | | | | | | | | | |
| | Polyglyceryl-3 Polydi- | 0.30 | 0.30 | 0.30 | 0.30 | 0.30 | 0.30 | 0.30 | 0.30 | 0.30 |
| | methylsiloxyethyl Di- | | | | | | | | | |
| | methicone | | | | | | | | | |
| | Acrylamide/Sodium | 0.60 | 0.60 | 0.60 | 0.60 | 0.60 | 0.60 | 0.60 | 0.60 | 0.60 |
| | Acryloyldimethyltaurate | | | | | | | | | |
| | Copolymer (and) | | | | | | | | | |
| | Isohexadecane (and) | | | | | | | | | |
| | Polysorbate 80 | | | | | | | | | |
| | Organic micro- | 2.00 | 4.00 | 6.00 | 8.00 | 10.00 | 14.00 | 16.0 | 18.0 | 20.0 |
| | particulate UV absorber | | | | | | | | | |
| | of formula (3), (5) or (7) | | | | | | | | | |
| | Ammonium | 0.65 | 0.65 | 0.65 | 0.65 | 0.65 | 0.65 | 0.65 | 0.65 | 0.65 |
| | Acryloyldimethyltaurate/ | | | | | | | | | |
| | VP Copolymer | | | | | | | | | |
| | Sodium Chloride | 0.10 | 0.10 | 0.10 | 0.10 | 0.10 | 0.10 | 0.10 | 0.10 | 0.10 |
| | Aqua | Qs to | Qs to | Qs to | Qs to | Qs to | Qs to | Qs to | Qs to | Qs to |
| | | 100 | 100 | 100 | 100 | 100 | 100 | 100 | 100 | 100 |

| | | | | | | | | | |
|---|---|---|---|---|---|---|---|---|---|
| In vitro SPF measured | 39.2 | 37.7 | 23.3 | 35.5 | 16.9 | 24.2 | 20.5 | 16.9 | 13.8 |
| according the method | | | | | | | | | |
| described | | | | | | | | | |
| UVA PF measured according | 12.9 | 11.6 | 10.3 | 16.6 | 11.1 | 12.8 | 10.5 | 11.4 | 10.1 |
| the method described | | | | | | | | | |

### EXAMPLE B21: Si/W Sun Cream

### Manufacturing Instruction:

Part A is prepared by incorporating all ingredients, then stirred under moderate speed. Part B is prepared and heated to 40° C. At this temperature, part A is poured into part B under progressive stirring speed till an homogeneous cream appears.

1. A method of preparing a composition, comprising (a) a micronised insoluble organic UV absorber, which method comprises grinding the insoluble organic UV absorber, in coarse particle form, in a grinding apparatus, in the presence of a grinding aid selected from the groups
(b₁) carboxylic acids and their salts;
(b₂) fatty acid esters
(b₃) alkyl phosphates or phosphoric acid esters;
(b₄) ethoxylated carboxylic acids or polyethyleneglycol (PEG) esters;
(b₅) fatty alcohol polyethyleneglycol (PEG) ethers of fatty alcohols;
(b₆) polyethylene/polypropylene -glycol ethers of saturated or unsaturated C₄-C₂₈ fatty;
(b₇) ethoxylated alkylphenols or ethoxylated alkylphenyl ethers;
(b₈) esters of polyol and mono-, di- or tri-glycerides;
(b₉) esters of fatty acids and saccharose;
(b₁₀) sorbitan mono- and di-esters of saturated and/or unsaturated C₆-C₂₂ fatty acids and ethylene oxide groups; and
(b₁₁) surfactants which are mainly acting as detergent or cleansing agents.

2. A method according to claim 1, wherein the micronised insoluble organic UV absorber is selected from-the compounds of formula (1) 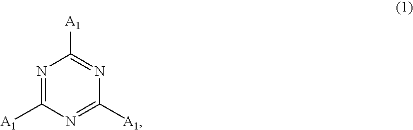
wherein
A is a radical of formula 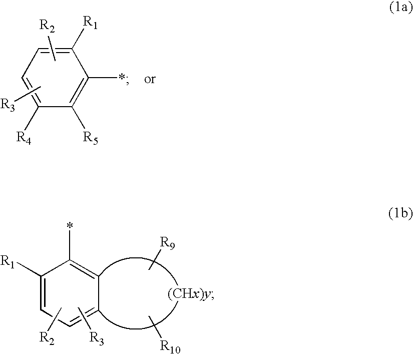
R₁ and R₅ independently from each other are hydrogen; C₁-C₁₈alkyl; or C₆-C₁₂aryl;
R₂, R₃ and R₄ independently from each other are hydrogen; or a radical of formula 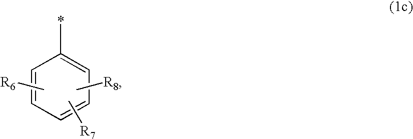
wherein at least one of the radicals R₂, R₃ and R₄ are a radical of formula (1c);
R₆, R₇, R₈, R₉ and R₁₀ independently from each other are hydrogen; hydroxy; halogen; C₁-C₁₈alkyl; C₁-C₁₈alkoxy; C₆-C₁₂aryl; biphenylyl; C₆-C₁₂aryloxy; C₁-C₁₈alkylthio; carboxy; —COOM; C₁-C₁₈-alkylcarboxyl; aminocarbonyl; mono- or di-C₁-C₁₈alkylamino; C₁-C₁₀acylamino; or —COON;
M is an alkali metal ion;
x is 1 or 2; and
y is a number from 2 to 10.

3. A method according to claim 2, wherein the micronized insoluble UV absorber is selected from the-compounds of formula (2)

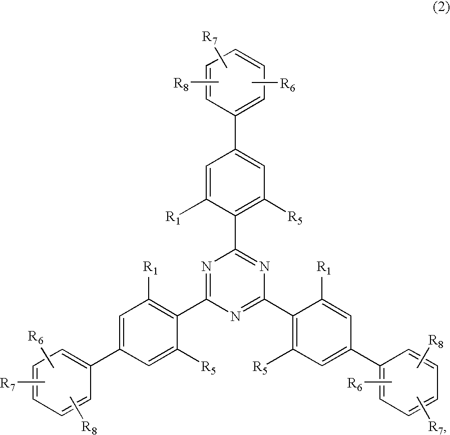

4. A method according to claim 3, wherein R₁ and R₅ are hydrogen.

5. A method according to claim 4, wherein R₆ and R₈ are hydrogen.

6. A method according to claim 5, wherein
R₇ is hydrogen; hydroxy; C₁-C₅alkyl; C₁-C₅alkoxy; —COOM; —COOH; or COOR₁₀;
M is an alkali metal ion; and
R₁₀ is C₁-C₅alkyl.

7. A method according to claim 6, wherein the micronized insoluble UV absorber is of formula (3)

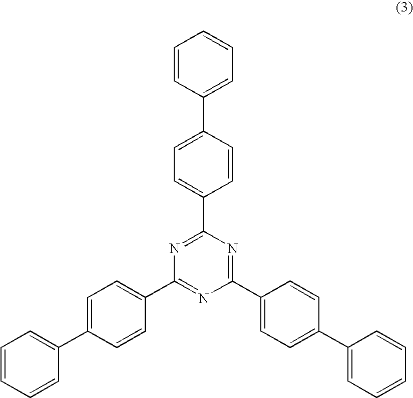

8. A method according to claim 1, wherein the micronized insoluble UV absorber is selected from compounds of formula (4) 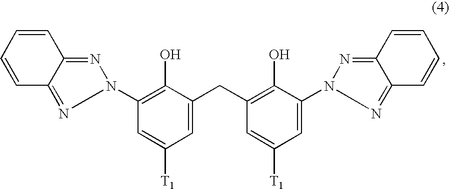
wherein
T₁ is C₁-C₁₈alkyl, which is optionally substituted by phenyl.

9. A method according to claim 8, wherein
T₁ is C₁-C₈alkyl.

10. A method according to claim 8, wherein the micronized insoluble UV absorber corresponds to formula (5)

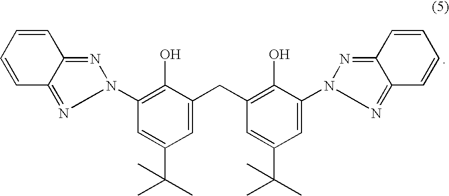

11. A method according to claim 1, wherein the micronized insoluble UV absorber is selected from compounds of formula (6) 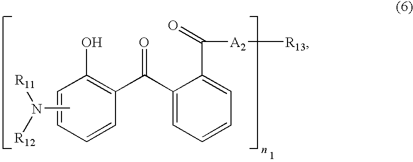
wherein
R₁₁ and R₁₂ independently from each other are C₁-C₂₀alkyl; C₂-C₂₀alkenyl; C₃-C₁₀cycloalkyl; C₃-C₁₀cycloalkenyl; or R₁₁ and R₁₂ together with the linking nitrogen atom form a 5- or 6-membered heterocyclic ring;
n₁ is a number from 1 to 4;
when n₁=1,
R₁₃ is a saturated or unsaturated heterocyclic radical; hydroxy-C₁-C₅alkyl; cyclohexyl optionally substituted with one or more C₁-C₅alkyl; or phenyl optionally substituted with a heterocyclic radical, aminocarbonyl or C₁-C₅alkylcarboxy
when n₁ is 2,
R₁₃ is an alkylene-, cycloalkylene, alkenylene or phenylene radical which is optionally substituted by a carbonyl- or carboxy group; a radical of formula *—CH₂—C≡C—CH₂—* or R₁₃ together with A₂ forms a bivalent radical of the formula 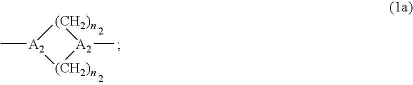
wherein
n₂ is a number from 1 to 3;
when n₁ is 3,
R₁₃ is an alkantriyl radical;
when n₁ is 4,
R₁₃ is an alkantetrayl radical;
A₂ is —O—; or —N(R₁₅)—; and
R₁₅ is hydrogen; C₁-C₅alkyl; or hydroxy-C₁-C₅alkyl.

12. A method according to claim 11, wherein the micronized insoluble UV absorber corresponds to the formula (7)

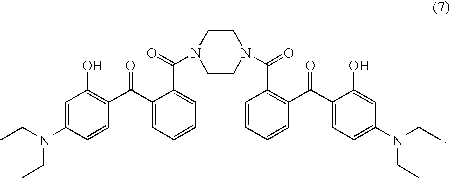

13. A method according to claim 1, wherein the grinding aids are selected from
(b₁) Sodium Lauroyl Lactylate,
(b₂) Isocetyl isostearate, or glycol oleate,
(b₃) DEA-oleth-3 phosphate,
(b₄) PEG-20 Laurate,
(b₅) Ceteth-10, Laureth-7 or PEG-10 Behenyl Ether (Beheneth-10),
(b₇) PEG-10 Nonyl Phenyl ether,
(b₈) PEG-10 polyglyceryl-2 laurate,
(b₉) PEG-120 Methyl Glucose Dioleate or Polyglyceryl-3 Methylglucose Distearate,
(b₁₀) PEG-20 Sorbitan Isostearate, Polysorbate-80 or PEG/PPG—125/30 coplymer, and
(b₁₁) Sodium dicocoylethylene diamine PEG-15 sulfate or Dimethicone PEG/PPG—7/4 Phosphate.

14. A method according to claim 1 in which the micronised insoluble organic UV absorber so obtained has a mean particle size in the range of from 0.01 to 2.0μ.

15. A method according to claim 1 in which the micronised insoluble organic UV absorber has been produced by grinding it, in coarse particulate form, in a grinding apparatus, until the insoluble organic UV absorber has been converted into micronised form.

16. A method according to claim 15 in which the grinding apparatus is a jet, ball, vibration or hammer mill.

17. A composition comprising a micronised insoluble organic UV absorber when produced by a method according to claim 1.

18. A sunscreen composition comprising
(a) 0.1 to 25% by weight of a micronised insoluble organic UV absorber dispersion according to claim 17; and optionally
(b) a cosmetically acceptable carrier.

19. A composition according to claim 18 in which the micronised insoluble organic UV absorber is used together with one or more further UV absorbers which are conventionally used in cosmetic compositions for the protection of human skin against UV radiation.

20. A composition according to claim 18, wherein
(c) a rheology modifier is additionally used.

21. A composition according to claim 20, wherein component (c) is selected from Xanthan Gum, amorphous Silica and modified Starch.

22. Composition according to claim 20, wherein the rheology modifier (c) is used in a concentration of 0.1 to 10% b.w. of the composition.
